(19) United States
(12) Patent Application Publication
(10) Pub. No.: US 2024/0034730 A1
(43) Pub. Date: Feb. 1, 2024

(54) PYRAZOLO DERIVATIVES AS HUMAN DIHYDROOROTATE DEHYDROGENASE (HDHODH) INHIBITORS FOR USE AS ANTIVIRALS

(71) Applicant: DRUG DISCOVERY AND CLINIC S.R.L., Torino (IT)

(72) Inventors: Donatella Boschi, Torino (IT); Marta Giorgis, Torino (IT); Marco Lucio Lolli, Torino (IT); Giovanni Martinelli, Mozzecane (Verona) (IT); Giuseppe Saglio, Fubine (Alessandria) (IT)

(21) Appl. No.: 18/036,867

(22) PCT Filed: Nov. 12, 2021
(86) PCT No.: PCT/EP2021/081467

§ 371 Date: May 12, 2023

(30) Foreign Application Priority Data
Nov. 13, 2020 (IT) 102020000027251

(51) Int. Cl. C07D 471/04; A61P 31/14

(52) U.S. Cl. CPC C07D 471/04; A61P 31/14

(57) ABSTRACT

The invention relates to hDHODH inhibitors, in particular 2-hydroxypyrazolo[1,5-a]pyridine derivatives, for use as antiviral agents. The hDHODH inhibitors for use according to the invention are effective as inhibitors of the replication of a wide spectrum of both DNA and RNA viruses, including, i.a., Herpes simplex virus 1 (HSV-1), Herpes simplex virus 2 (HSV-2), Influenza A virus, Influenza B virus, Respiratory syncytial virus (RSV), Severe acute respiratory syndrome coronavirus 1 (SARS-CoV-1), Severe acute respiratory syndrome coronavirus 2 (SARS-CoV-2) and Middle East respiratory syndrome-related coronavirus (MERS-CoV).

### FIELD OF THE INVENTION

The present invention relates to human dihydroorotate dehydrogenase (hDHODH) inhibitors for use as antivirals. The hDHODH inhibitors for use according to the invention are effective as antivirals by triggering in the host cell pyrimidine starvation due to inhibition of hDHODH. The hDHODH inhibitors for use according to the invention are effective against a broad spectrum of both RNA and DNA viruses, including, inter alia, SARS-CoV-2 and other important human viral pathogens.

### BACKGROUND OF THE INVENTION

The social and economic long terms effects of the COVID-19 pandemic are unpredictable, however strategic decisions made now and in the coming months will be some of the most important made in generations. After many outbreaks of emerging and re-emerging RNA viruses, such as SARS, Influenza virus, MERS, Ebola, and Zika, strategic actions recommended to better prepare for other viral disease outbreaks, such as investments on Broad Spectrum Antiviral Agents (BSAAs), remain inadequate and this has led to the present crisis scenario. Among BSAAs, which are advantageously able to be rapidly adapted from a pandemic event to another, Host-Targeting Antivirals (HTAs) work by interfering with cellular biochemical pathways commonly used by different viruses for the replication, thus overcoming virus specificity and potential viral mutagenesis.

Human dihydroorotate dehydrogenase (hDHODH, EC 1.3.99.11) present in the inner mitochondrial membrane, is a flavin-dependent enzyme involved in de novo pyrimidine biosynthesis. It catalyzes the rate-limiting step in de novo pyrimidine biosynthesis, which converts dihydroorotate (DHO) to orotate (ORO). hDHODH has already been validated as a therapeutic target for the treatment of autoimmune diseases, such as rheumatoid arthritis and multiple sclerosis. Furthermore, hDHODH has also recently been identified as a relevant target in the treatment of triple-negative breast cancer,¹ PTEN-mutant tumors,² KRAS-driven tumors,³ acute myelogenous leukemia (AML) and viral infection.⁴ The connection with AML has paved the way for completely new perspectives in the treatment of the disease as well as in the hDHODH field. Presumably, all of these diverse malignancies converge on a similar pathway of metabolic reprogramming to drive their dependence on pyrimidine synthesis and sensitivity to DHODH-inhibition.

The inventors recently identified and characterized a novel class of hDHODH inhibitors⁵, including, inter alia, compound 1 (named as compound 4 in ref. 6), which has a particularly elevated potency toward the hDHODH enzyme (hDHODH IC₅₀ 1.2 nM) and excellent drug-like properties.

### SUMMARY OF THE INVENTION

The inventors have now unexpectedly found that compound 1 and its derivatives illustrated in formulae (I) to (V) below are also potent and broad-spectrum antiviral agents, including anti-SARS-CoV-2. The research carried out by the present inventors was dedicated to investigate the use of compound 1 and its derivatives as Broad Spectrum Antiviral Agent (BSAA), as well as to refine the Structure Activity Relationship (SAR) of this class of hDHODH inhibitors. As it will be illustrated in detail in the following, the tested hDHODH inhibitors for use according to the present invention advantageously showed high activity in vitro. In particular, compound 1 is superior to brequinar in terms of antiviral potency and safety profile, as it is able to block viral replication at concentrations that are 1 log digit lower than those obtained with brequinar. Given its ability to inhibit SARS-CoV-2 replication with EC₅₀ 74 nM and an incredibly effective SI (>7900, CC₅₀>500 μM), compound 1 has one of the most potent and safer in vitro profile so far obtained against SARS-CoV-2 replication in E6 cells. Compound 1 was also investigated as BSAA against other viruses and showed similar potencies.

To pave the way to future preclinical and clinical applications, the inventors also carried out some studies concerning the pKs, the half-life per os and i.v., the in vivo toxicity and metabolism, as well as the in vitro activity against some selected pathogenic viruses of compounds for use according to the invention. Such studies are illustrated in the following. The compounds were tested against the following viruses: Herpes simplex 1 and 2, Influenza Virus and some pathogenic viruses for the respiratory tract such as Respiratory Syncytial Virus (RSV), one of the main causes of infant's hospitalization and mortality, as well as severe acute respiratory syndrome corona virus 2 (SARS-CoV-2), belonging to the same family of viruses responsible for severe acute respiratory syndrome (SARS) and Middle East respiratory syndrome (MERS).

### DETAILED DESCRIPTION OF THE INVENTION

The present invention relates to a class of hDHODH inhibitors, based on an unusual carboxylic group bioisostere, the 2-hydroxypyrazolo[1,5-a]pyridine, for use as antiviral agents, i.e. as inhibitors of virus replication.

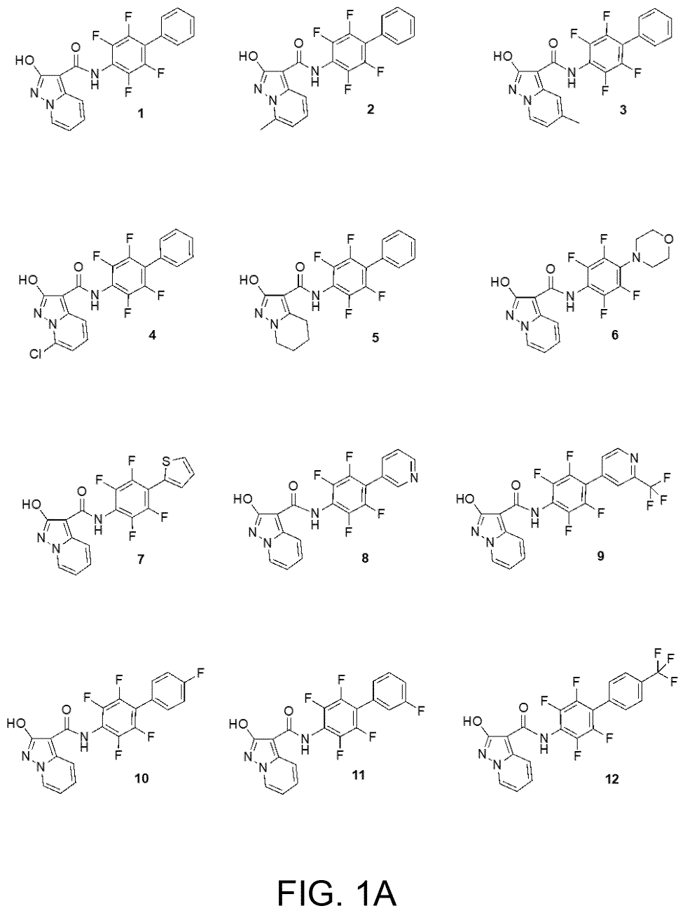

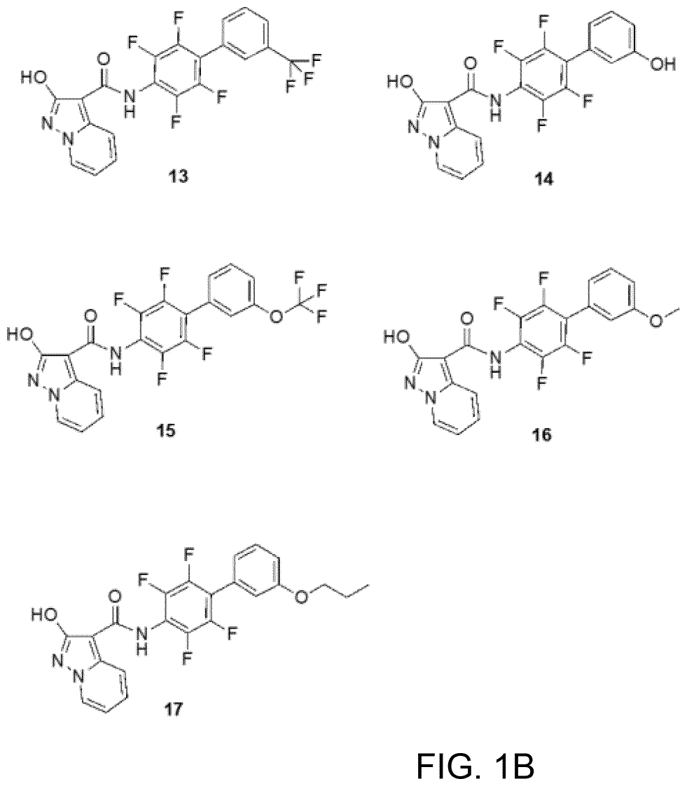

2-Hydroxypyrazolo[1,5-a]pyridine is a system that is still relatively unexplored in the literature. The present description reports its scaffold-hopping use as a bioisostere of a carboxylic function (often present in DHODH inhibitor structures) in preparing two series of derivatives. In the first series, besides investigating the moiety itself (compounds 2-5, FIG. 1A), the inventors have also investigated the effect of introducing a chloro and a methyl group into the pyridine ring in order to improve its lipophilic interaction with hDHODH subsite 4 (compounds 2, 3 and 4, FIG. 1) or replacing a pyridine with a tetrahydrogenated pyridine (pyperidine) moiety as in compound 5. The second ring of the biphenylic scaffold was also subject of investigation by inserting either polar (compounds 6, 8, 9, 14) or lipophilic (compounds 7, 10-13, 15-17) moieties.

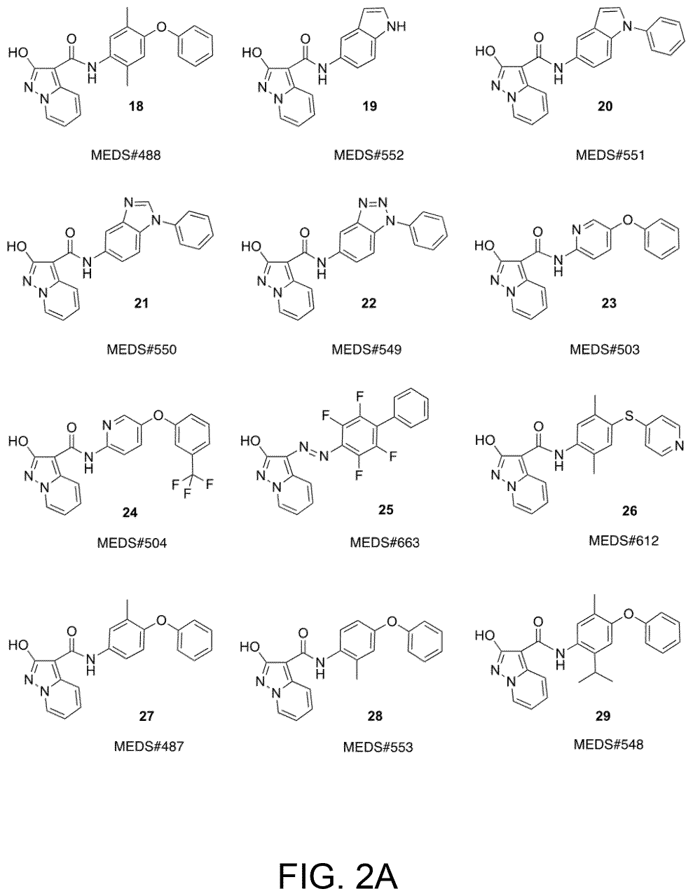

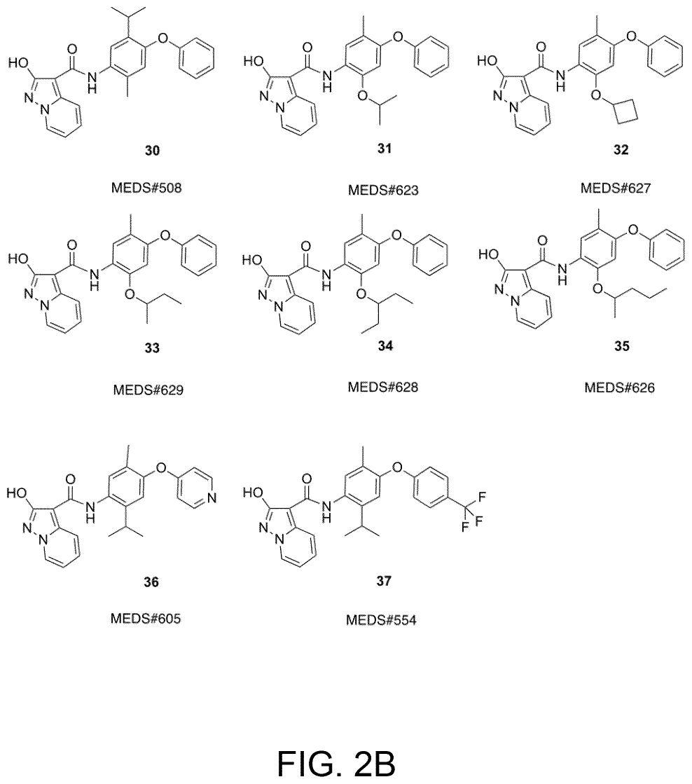

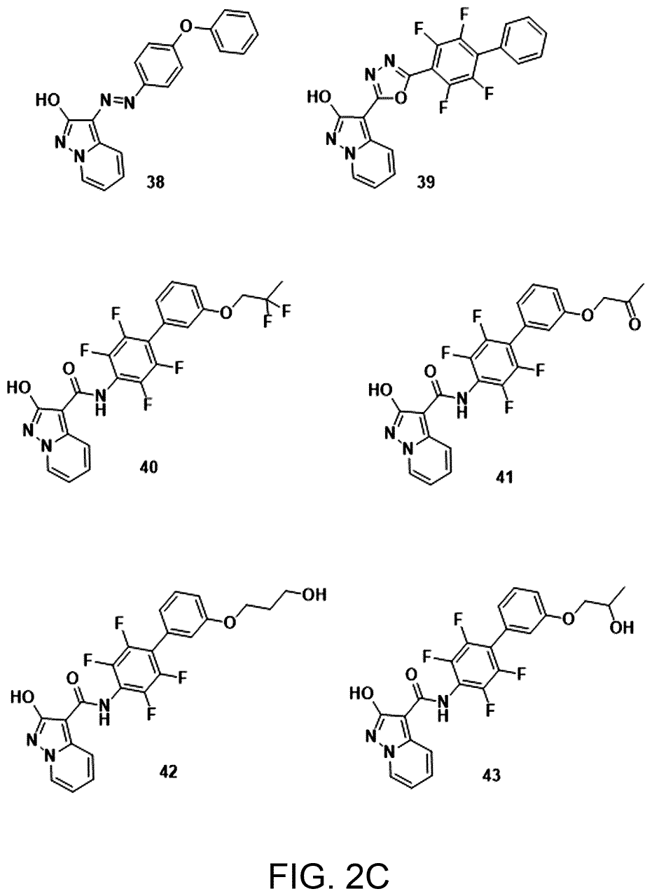

In the second series (compounds 18, 23, 24, 26-37, FIGS. 2A and 2B), the inventors have replaced the biphenyl substituent with a more flexible diphenyl ether in order to improve its pharmacokinetic properties and provide more drug-like compounds. The theoretical design, synthesis, SAR, biological assays, physicochemical characterization and preliminary ADME profiles of the compounds are presented and discussed fully hereinafter.

Accordingly, a first aspect of the present invention is a 2-hydroxypyrazolo[1,5-a]pyridine scaffold-based hDHODH inhibitor of Formula (I), (II), (III), (IV) or (V) illustrated below, for use in inhibiting viral replication or for use as an antiviral agent.

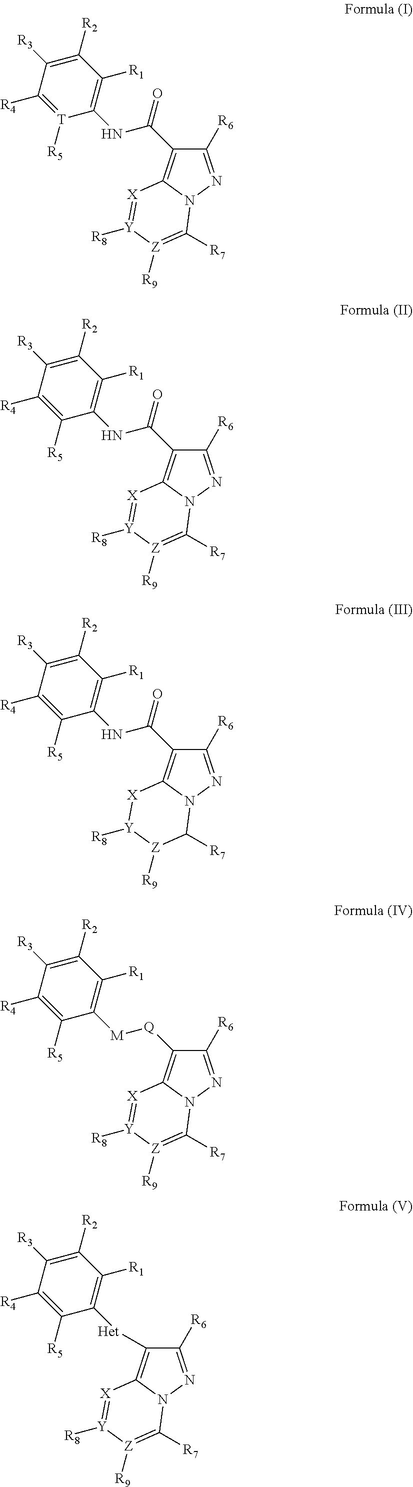

In Formulae (I) to (V) above:
R₁, R₂, R₄ and R₅ are independently selected from a hydrogen atom, a halogen atom, a alkyl group, a alkyloxy group, a cycloalkyloxy group, an alkylthio group, a halo alkyl group, a halo alkyloxy group, a nitro group, a cyano group, and alkylamino group;
R₃ is selected from an optionally substituted phenyl group, heteroaryl group, pyridinyl group, piperidinyl group, phenoxy group, pyridinoxy group, piperidinyloxy group, phenylthio group, azinyl group, phenylsulfonyl group, phenylsulfinyl group, phenylsulfonylamino group, alkyl group, alkyloxy group, alkylthio group, halo alkyl group, and halo alkyloxy group;
R₇, R₈ and R₉ are independently selected from a hydrogen atom, a halogen atom, a nitro group, a cyano group, a halo alkyl group, a thio alkyl group, an amino alkyl group, a alkyl group, and a hydroxy alkyl group;
R₆ is selected from a alkyloxy group, a halogen atom, an acyloxy group, a monophosphate group, a hydroxyl group, a thiol group, an amino group, or a salt thereof;
X, Y and Z are independently selected from a carbon atom, a nitrogen atom, an oxygen atom and a sulfur atom, with the proviso that when one of X, Y or Z is nitrogen, oxygen or sulfur, the other two positions are carbon atoms;
in Formula (I), T is a carbon atom or a heteroatom like nitrogen atom, with the proviso that when T is a nitrogen atom, R₅ in Formula (I) does not exist;
in Formula (IV), M is selected from a carbon sp2 atom, a nitrogen sp3 atom, a nitrogen sp2 atom, a carbonyl group and a sulfonyl group;
in Formula (IV), Q is selected from a carbon sp2 atom, a carbonyl group, a thiocarbonyl group, a sulfonyl group, a polihalogenate-C2-alkylchain, a carbonylamino group, an aminocarbonyl group, a nitrogen sp2 atom and a nitrogen sp3 atom;
with the provisos that when M is a carbon sp2 atom, Q is a carbon sp2 atom; when M is a nitrogen sp3 atom, Q is selected from a carbonyl group, a thiocarbonyl group, a sulfonyl group, a polihalogenate-C₂-alkylchain, a carbonylamino group, an aminocarbonyl group; when M is a nitrogen sp2 atom, Q is a nitrogen sp2 atom; when M is a carbonyl group or sulfonyl group, Q is a nitrogen sp3 atom;
in Formula (V), Het is selected from azoles, such as an imidazole, a pyrazole, an oxazole, a thiazole, a triazole, an oxadiazole, a thiadiazole, a tetrazole.

According to a preferred embodiment, at least one of R₁, R₂, R₄ and R₅ is or contains a halogen atom. A preferred halogen atom is a fluorine atom (F). In a particularly preferred embodiment, all of R₁, R₂, R₄ and R₅ are fluorine (F).

Preferred alkyls in the definition of R₁, R₂, R₄, R₅ are C1-C6 alkyls, more preferably C1-C4 alkyls. Alkyls are either linear or branched.

Particularly preferred meanings for R₁, R₂, R₄ and/or R₅ are H (hydrogen), F (fluorine), Cl (chlorine), —CH₃, —CH(CH₃)₂, —O—CH(CH₃)₂, -o-cyclobutyl, —O—CH(CH₃)(CH₂CH₃), —O—CH(CH₂CH₃)₂, —O—CH(CH₃) (CH₂CH₂CH₃).

Preferred alkyls in the definitions of R₃ are C1-C12 alkyls. Alkyls are either linear or branched.

Particularly preferred meanings for R₃ are optionally substituted phenyl, phenoxy, thiophenol, morpholine, thiophene, pyridine and indole radicals. Suitable substituents are for example halogen atoms (e.g., F or Cl), alkyl or alkoxy groups (such as methyl, methoxy, ethyl ethoxy, propyl, propoxy); haloalkyl or haloalkoxy groups (such as trifluoroalkyl, trifluoroalkoxy, difluoroalkyl, difluoroalkoxy, fluoroalkyl, fluoroalkoxy, in which alkyl is preferably methyl, ethyl, propyl or butyl); —OH; oxyketones and oxyalcohols (such as oxypropanone, oxypropanol).

Preferred alkyls in the definitions of R₇, R₈ and R₉ are C1-C6 alkyls, more preferably C1-C4 alkyls. Alkyls are either linear or branched.

In the definition of R₆, if R₆ is OH or a monophosphate, preferred salts are Na⁺, K⁺ Ca or Cs²⁺ salts.

In all of the aforementioned embodiments, a preferred C₁-C₄ alkyl group as R₇ and R₈ is a methyl group while both X, Y and Z are carbon sp2 atoms.

Formula (Ia) below illustrates a preferred embodiment of Formula (I) in which X=Y=Z are sp² C atoms and R₉ is a proton:

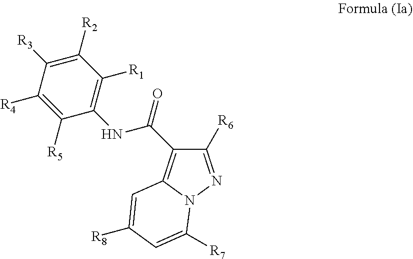

Preferred compounds falling within the scope of Formulae (I) and (Ia) are compounds 1-4, 6-18, 23-24, 26-37, 40-43 illustrated in FIGS. 1 and 2.

Formula (IIa) below illustrates a preferred embodiment of Formula (II) in which X=Y=Z are sp a C atoms and R₉ is a proton:

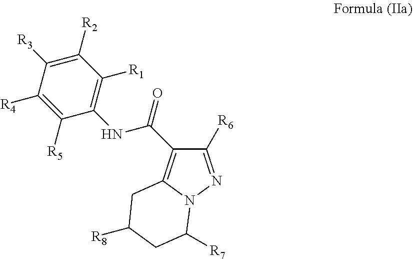

Preferred compounds falling within Formulae (I) to (V) are compounds 1 to 43 illustrated in FIGS. 1A, 1B, and FIGS. 2A, 2B and 2C.

Particularly preferred inhibitors for use according to the invention are compounds 1 and 17, having the structural formulae depicted below:

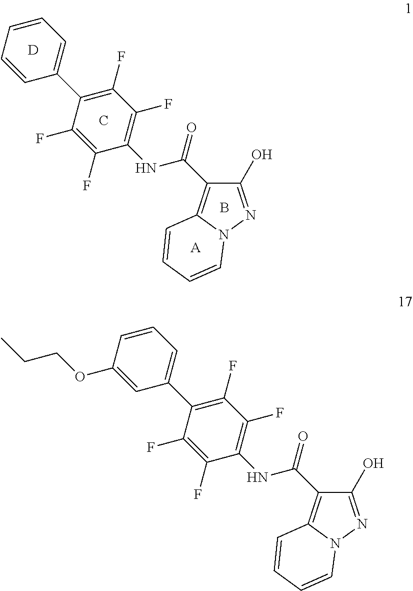

Particularly preferred compounds 1 (also designated as “MEDS433”) and 17 show brequinar-like hDHODH potency levels in vitro and are superior in terms of antiviral potency and selectivity blocking the viral replication at concentrations that are 1 log digit lower than those achieved in experiments with brequinar.

A second aspect of the present invention is an antiviral pharmaceutical composition comprising a 2-hydroxypyrazolo[1,5-a]pyridine scaffold-based hDHODH inhibitor of Formulae (I) to (V) as defined above as the antiviral agent, and a pharmaceutically acceptable carrier, excipient and/or diluent.

A third aspect of the present invention is a 2-hydroxypyrazolo[1,5-a]pyridine scaffold-based hDHODH inhibitor of the general formulae (I) to (V) as defined above for use in the therapeutic treatment of a virus infection in a subject, wherein the virus is preferably pathogenic.

In all of the aforementioned aspects of the present invention, the virus is a DNA virus or an RNA virus. Preferably, the virus is selected from the group consisting of Herpesviridae, Orthomyxoviridae, Paramyxoviridae and Coronaviridae. More preferably, the virus is selected from the group consisting of Herpes simplex virus 1 (HSV-1), Herpes simplex virus 2 (HSV-2), Influenza A virus, Influenza B virus, Respiratory syncytial virus (RSV), Severe acute respiratory syndrome coronavirus 1 (SARS-CoV-1), Severe acute respiratory syndrome coronavirus 2 (SARS-CoV-2) and Middle East respiratory syndrome-related coronavirus (MERS-CoV).

The following experimental part is provided by way of illustration only and is not intended to limit the scope of the invention as defined by the appended claims.

In the experimental part, reference is made to the following figures:

FIGS. 1A, 1B and FIGS. 2A, 2B, 2C showing the structures of compounds 1-43 which are the preferred compounds for use according to the invention.

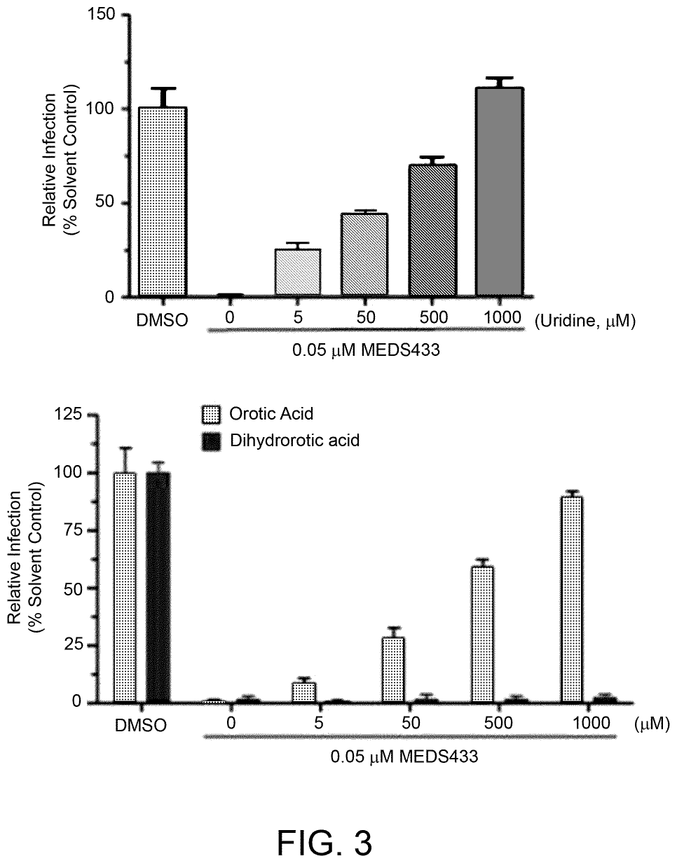

FIG. 3 includes two diagrams showing that the antiviral activity of compound 1 against RSV is rescued by uridine and orotic acid.

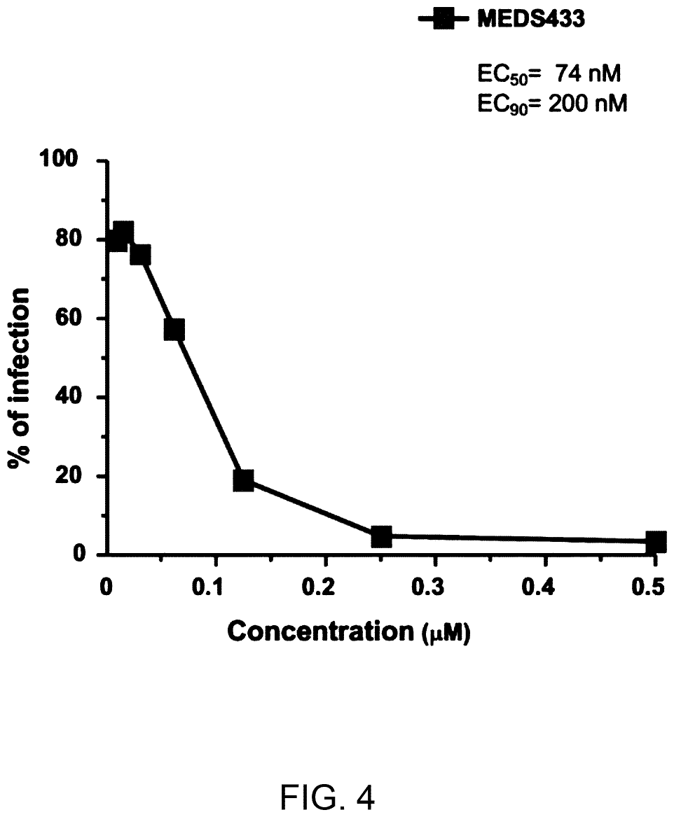

FIG. 4 is a graph showing that compound 1 inhibits SARS-CoV-2 replication in Vero E6 cells.

### EXPERIMENTAL PART

### Synthesis Schemes

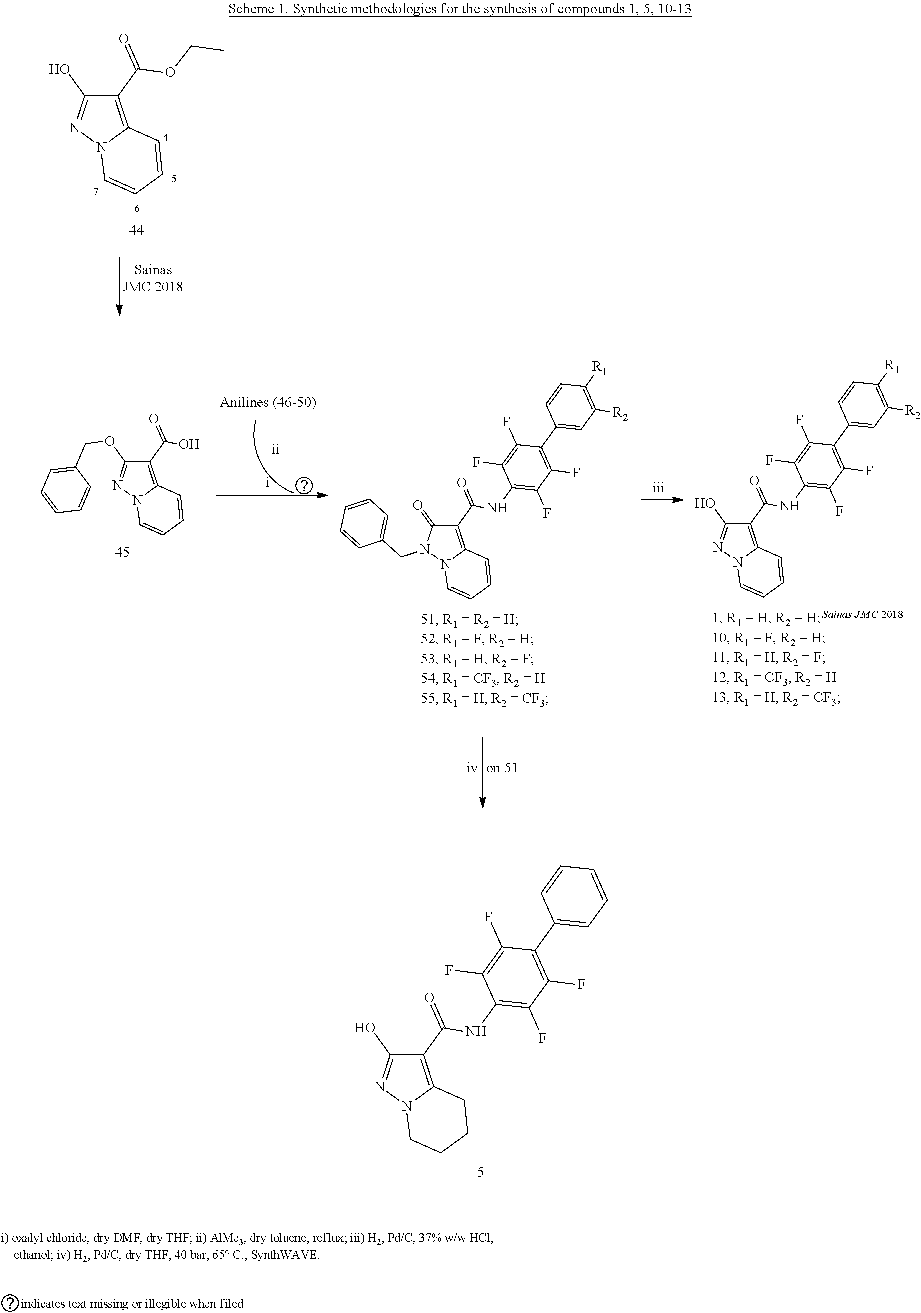

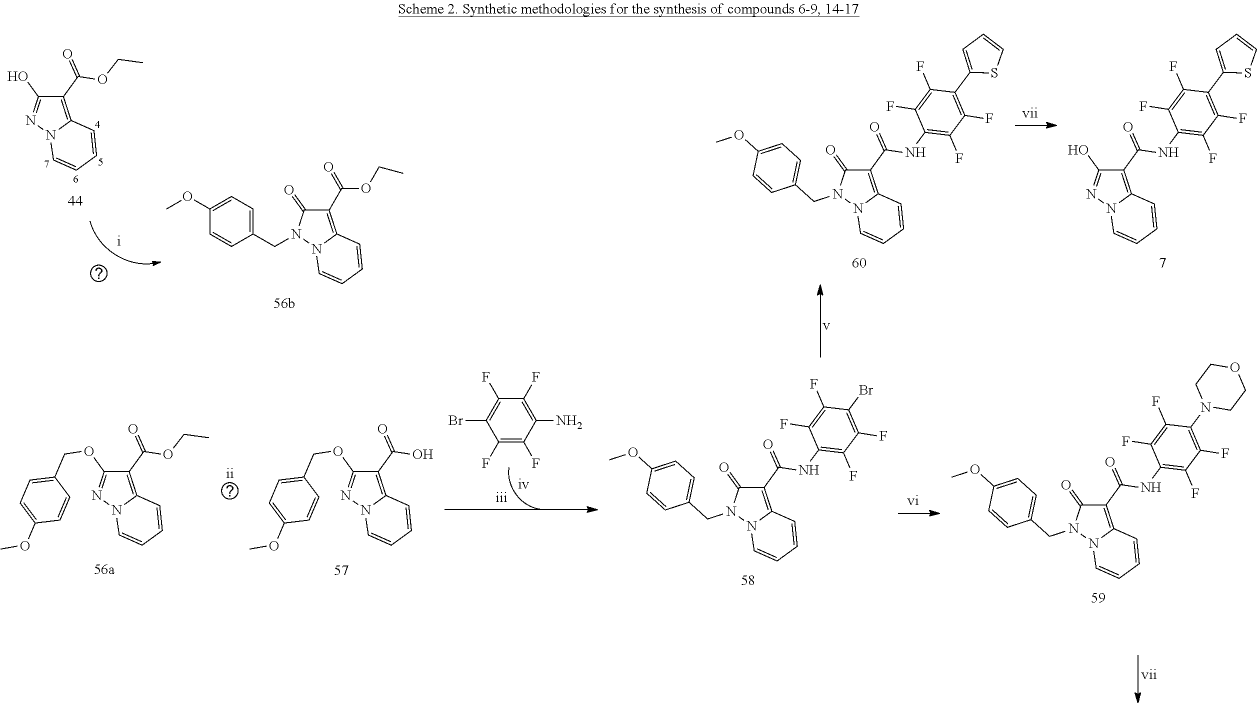

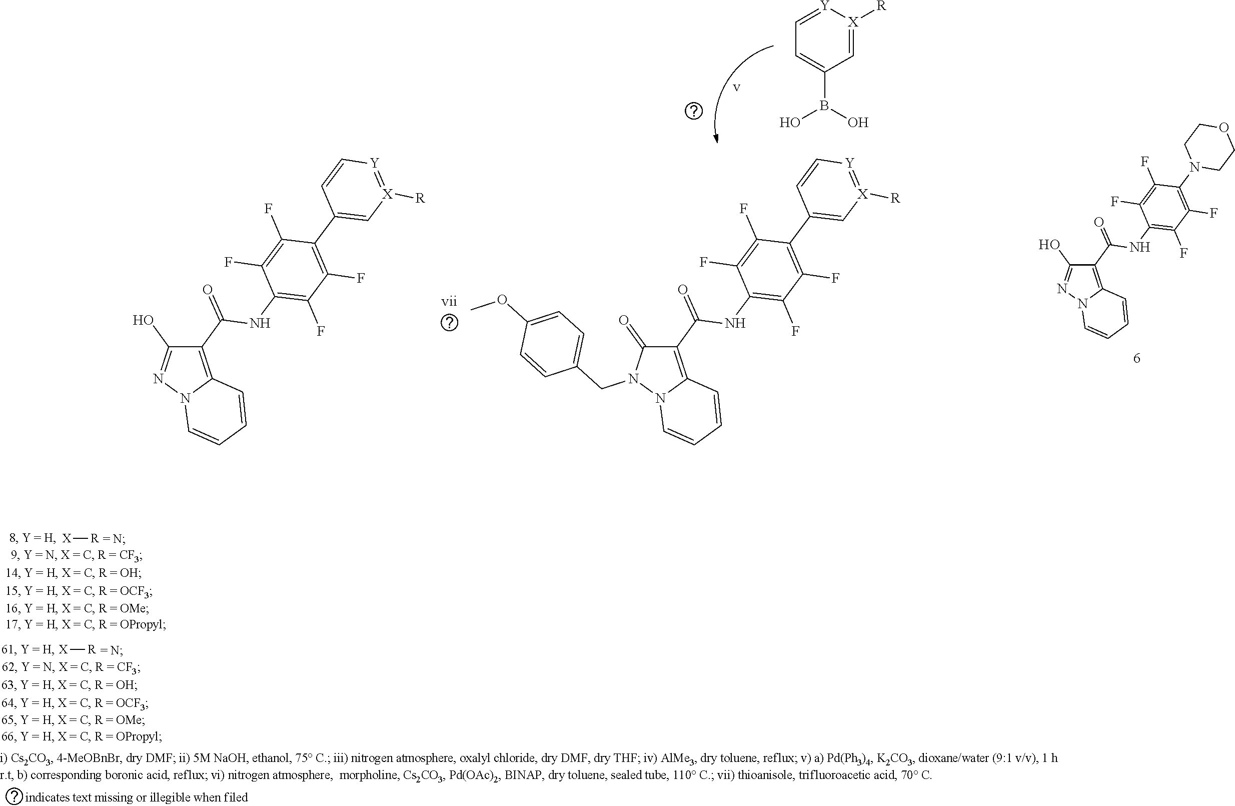

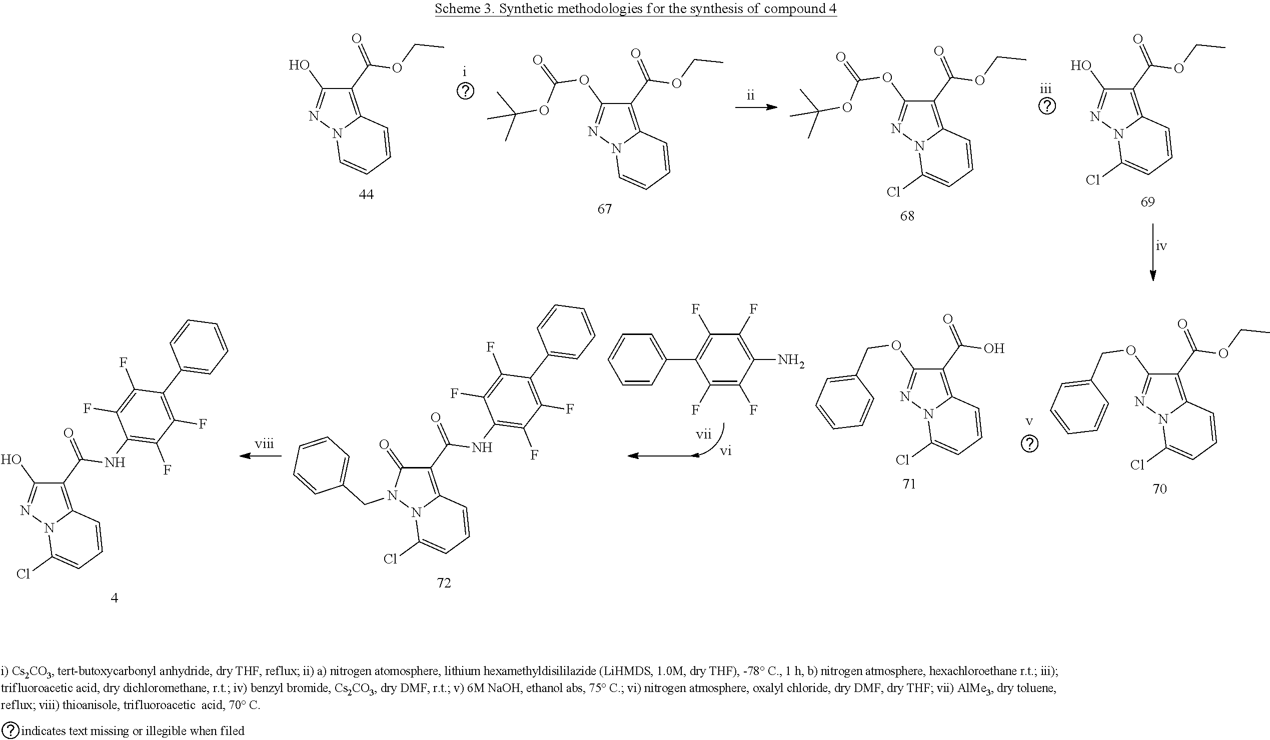

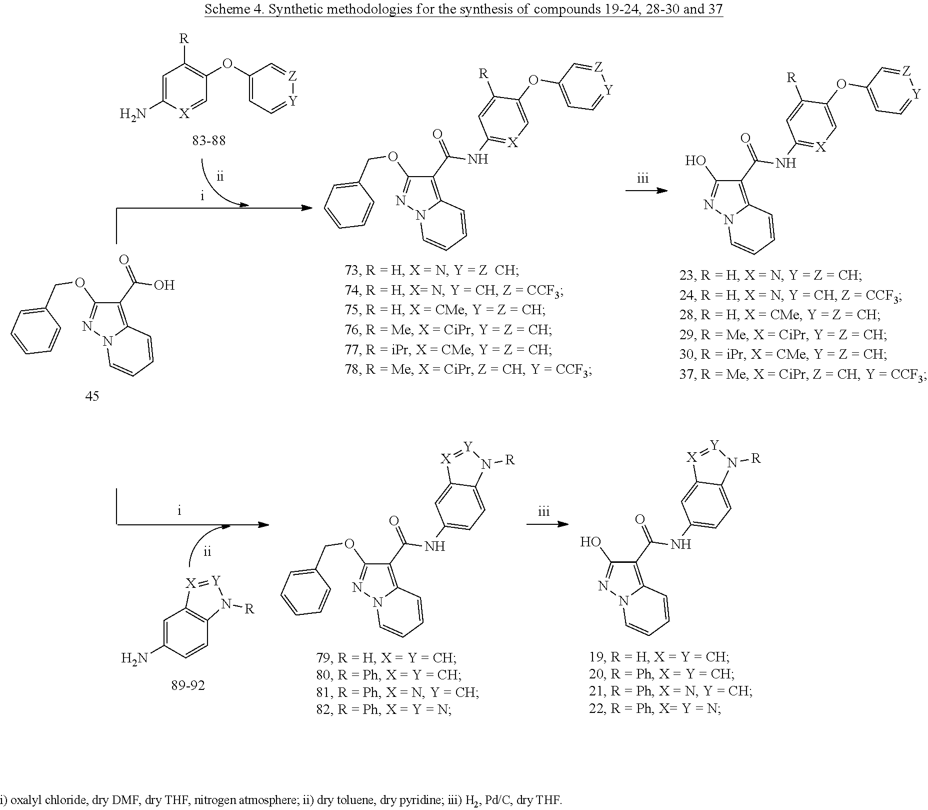

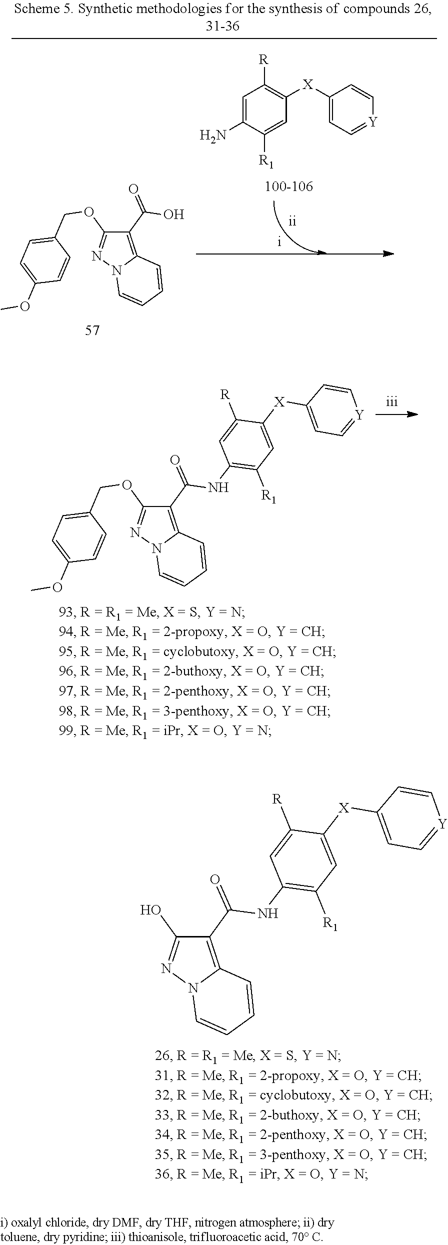

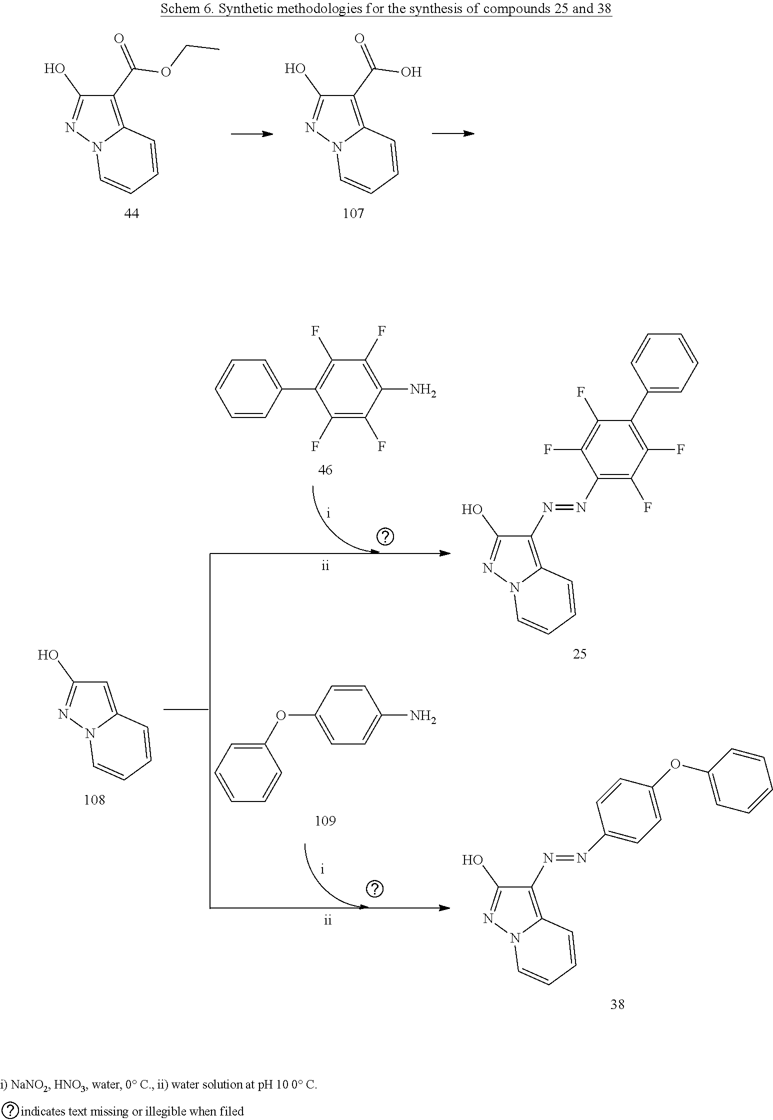

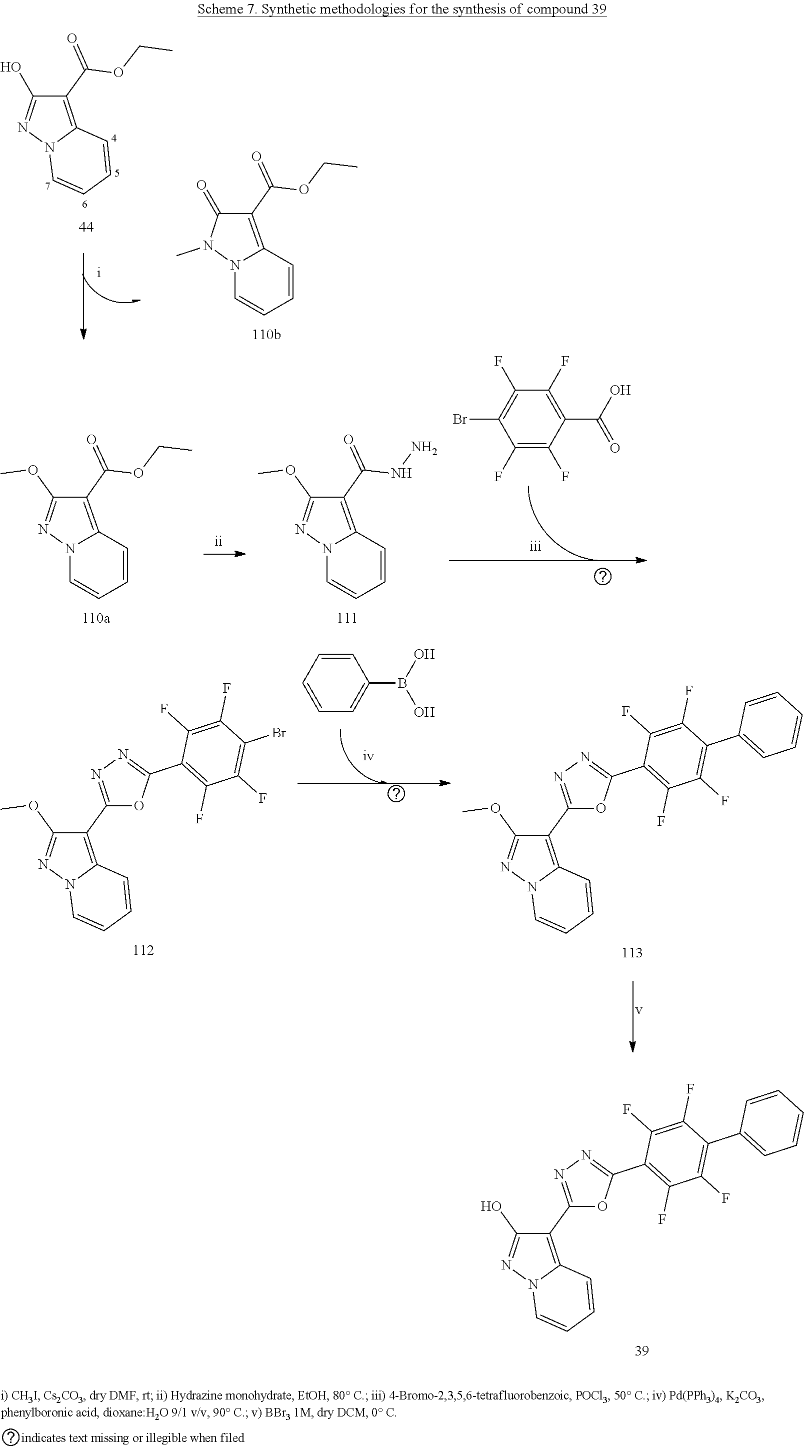

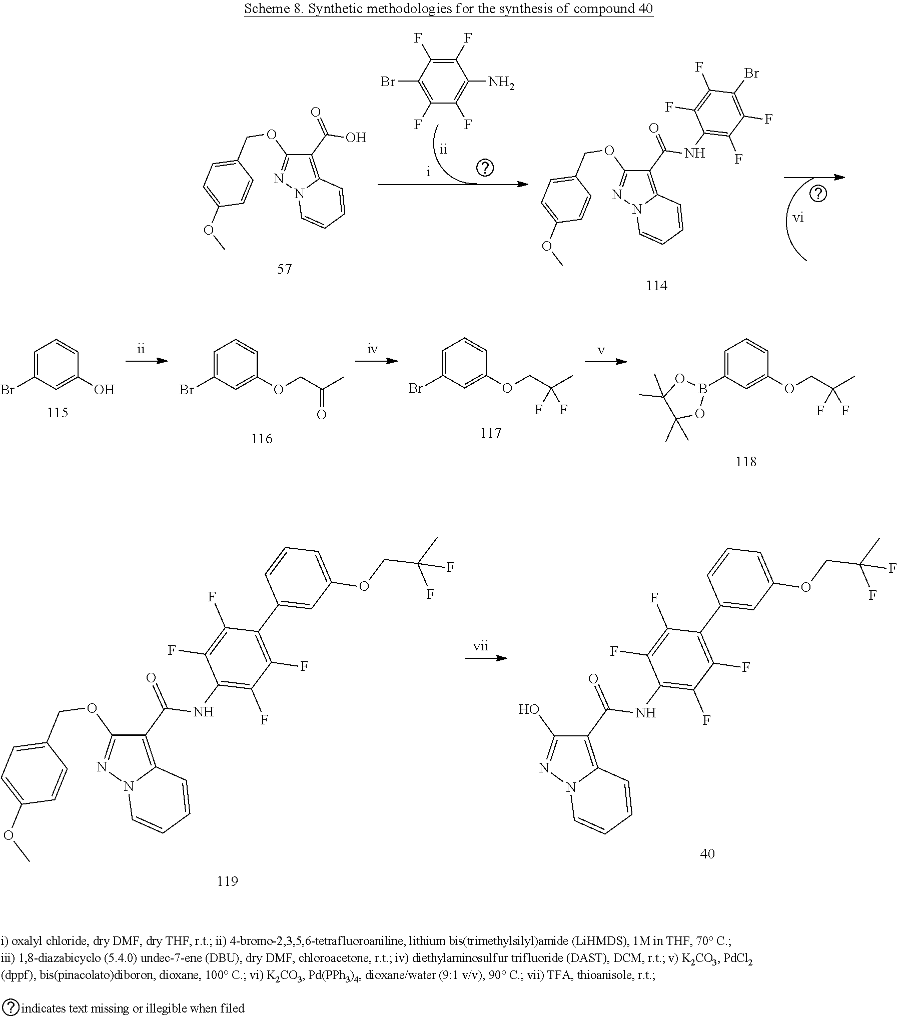

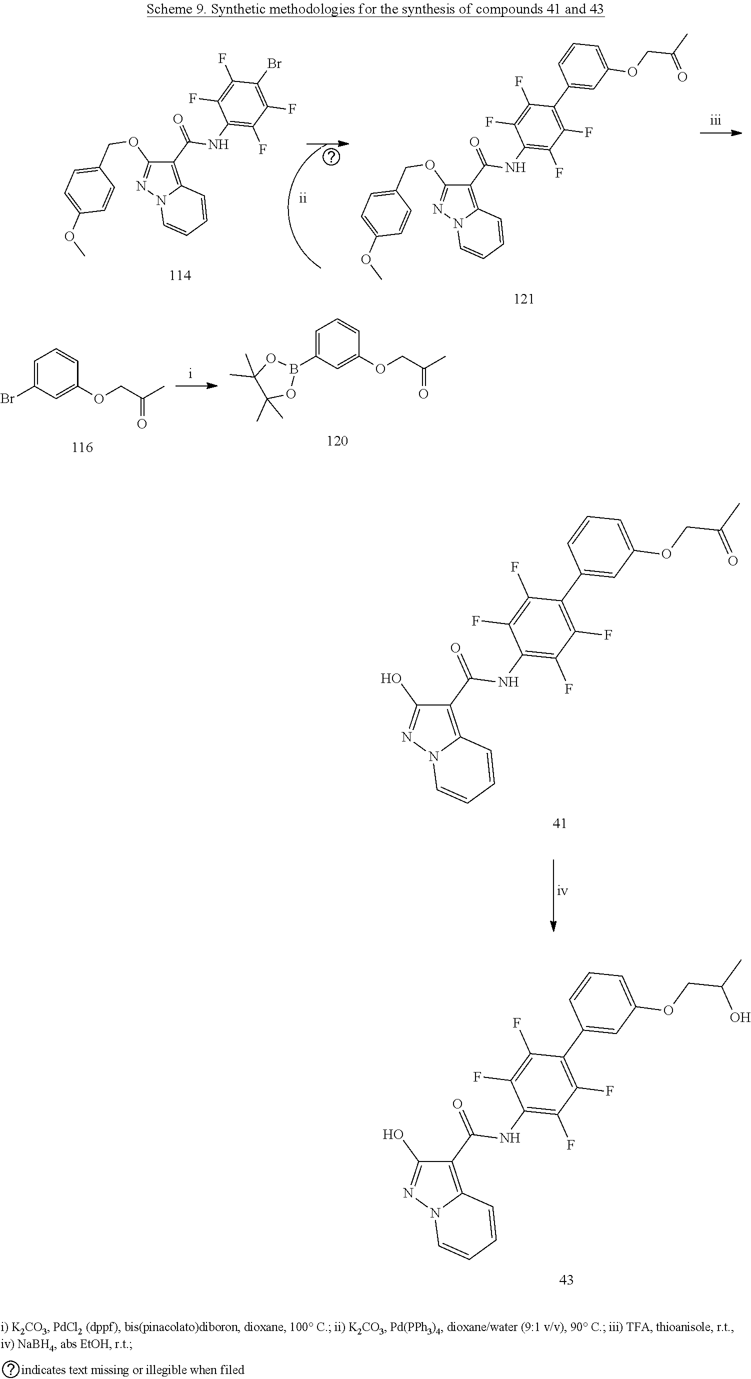 [SMILES: COc1ccc(COc2nn3ccccc3c2C(=O)Nc2c(F)c(F)c(Br)c(F)c2F)cc1 ; COc1ccc(COc2nn3ccccc3c2C(=O)Nc2c(F)c(F)c(-c3cccc(OCC(C)=O)c3)c(F)c2F)cc1 ; CC(=O)COc1cccc(Br)c1 ; CC(=O)COc1cccc(B2OC(C)(C)C(C)(C)O2)c1 ; CC(=O)COc1cccc(-c2c(F)c(F)c(NC(=O)c3c(O)nn4ccccc34)c(F)c2F)c1 ; CC(O)COc1cccc(-c2c(F)c(F)c(NC(=O)c3c(O)nn4ccccc34)c(F)c2F)c1]

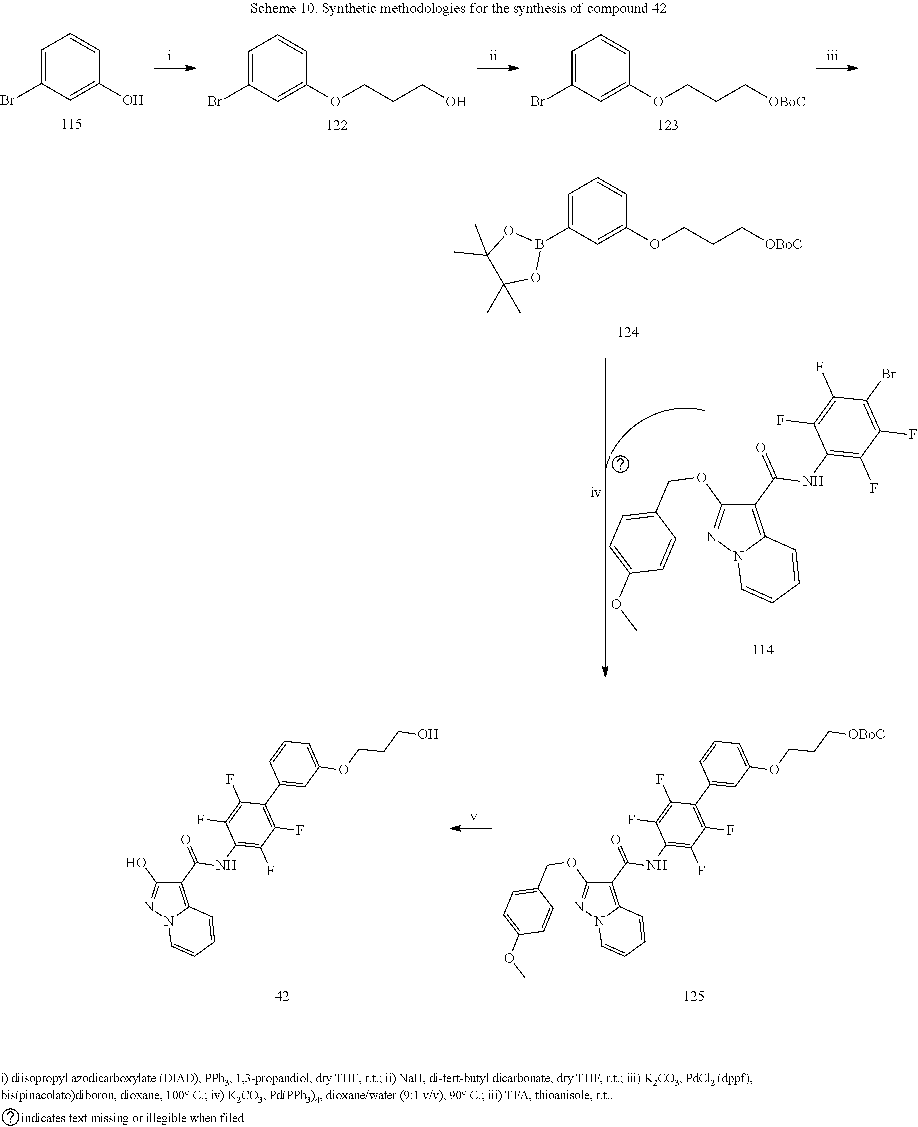

### Results and Discussion

### Chemistry: Synthesis of Compounds 2-43

For the syntheses of compounds 5 and 10-13, a chemical strategy already investigated to the lead compound 1 was used (see Scheme 1). The scheme starts from protected 2-hydroxypyrazolo[1,5-a]pyridine building block 45, this latter obtained by 44 in two steps⁶. From 45, the corresponding acyl chloride was obtained and used directly without further purification. Due to their poor reactivity with acyl chloride, each aniline (46-50) was converted into the more reactive dimethylaluminum amide, these latter were allowed to reach with the above acyl chloride obtaining the desired amides 51-55 in 31-40% yield range. Note how, during the coupling step the benzylic protecting group transposed from the exocyclic oxygen to the endocyclic N1 nitrogen the pyrazolo[1,5-a]pyridine system. During the synthesis of 1,⁶ the removal of the benzyloxy moiety for 51 was always effected by the presence of a side reaction that lead to traces of the reduced 5. On this occasion, 5 was obtained in 44% yield applying stronger catalytic hydrogenation conditions (40-bar) and using a SynthWAVE apparatus. To the opposite, to avoid such side reaction, compounds 52-55 were converted to desired target compounds 10-13 by applying room-pressure catalytic hydrogenation in presence of 37% w/w HCl.

For the synthesis of compounds 6-9 and 14-17, we designed a convergent synthetic approach with late-step Suzuki coupling of compound 58, as common intermediate (Scheme 2). Also in this case the scheme started from 2-hydroxypyrazolo[1,5-a]pyridine 44, that was protected with 4-methoxybenzyl bromide to afford the regioisomers 56a and 56b in ratio 61 and 27% respectively (see SI for the spectroscopical characterization of the two regioisosmers). The 4-methoxybenzyl protecting group can be easily removed in acidic conditions, applicable also to molecules containing sulphur atoms and pyridine ring, both known to poison metal catalyst during hydrogenation. Ester 56a was then hydrolyzed under basic conditions to obtain the corresponding acid 57 (quantitative yield), which were then used for the preparation of the common intermediate 58. Starting from acid 57, the corresponding acyl chloride was obtained via treatment with oxalyl chloride that was used without any further purification in the reaction with the dimethylaluminum amide of 2,3,5,6-tetrafluoro-4-bromoaniline affording the desired amide 58 in the 55% yield. Also in this case, transposition of the benzylic protecting group from the exocyclic oxygen to the endocyclic N1 nitrogen the pyrazolo[1,5-a]pyridine system was observed. Compound 58 was used as commune building block for the desired compounds 59, 60-66. Firstly, by applying a Buchwald-Hartwig coupling⁷, conditions with morpholine, was obtained 59 (59% yield), then by Suzuki reaction involving the corresponding boronic acids were obtained 60-66 (yield range: 70-94%). Compounds 59-66 were then converted to the desired targets 6-9, 14-17 by treatment with trifluoroacetic acid (TFA) in presence of thioanisole as scavenger.

For the synthesis of compound 4, a dedicated synthetic scheme was applied (Scheme 3). In this case, the hydroxyl group of 44 was 0-protected with Boc group to afford 67. Using lithium hexamethyldisilylazide on 67, the pyrazolo[1,5-a]pyridine moiety was selectively deprotonated on the 7 position; in the following, the quench of the lithium salt of 67 with hexachloroethane, employed as electrophile source of Cl⁺, afforded compound 68 in good yield⁸. In order to move on the reaction scheme and prepare the following coupling steps, the Boc group was ideally exchanged for a benzyl group. The Boc group was quantitatively removed under mild acid condition (TFA) obtaining the hydroxyazole 69 that was allowed to reach with benzyl bromide affording compound 70 (90% over two steps). It is worth to note that in this case, because of the presence of a chlorine in position 7, the endocyclic N1 isomer was obtained only in traces. The ester 70 was then hydrolyzed under basic conditions to obtain the corresponding acid 71 (quantitative yield) that was used for the preparation of the amide 72 using the above described conditions that involved the activation of the 2,3,5,6-tetrafluoro-4-phenylaniline as dimethylaluminum amide affording the desired amide 72 in the 38% yield. Compound 72 was then converted to desired target 4 by treatment with TFA in presence of thioanisole as scavenger.

The synthetic strategy used to produce the 2-aryloxypyrazolo[1,5-a]pyridine building blocks 45 and 57, which are useful in the syntheses of target compounds 19-24, 26 and 28-37, were achieved using procedures and conditions detailed in our previous publication 6 (Scheme 4). Starting from the acid 45, compounds 73-82 were prepared, via a known procedure (see Scheme 4), the corresponding acyl chloride was obtained via treatment with oxalyl chloride and used directly after drying without further purification. On the opposite of our earlier work dedicated to hDHODH, the acyl chloride, was allowed to react directly with the corresponding aniline (83-92) previously synthetized. The desired amides 73-82 were obtained in the 50-92% yield range. Moving forward, compounds 73-82 were then converted to desired target compounds 19-24, 28-30 and 37 by applying room-pressure catalytic hydrogenation. Starting from the acid 57, compounds 93-99 were prepared, (see Scheme 5), the corresponding acyl chloride was obtained via treatment with oxalyl chloride and used directly after drying without further purification. The acyl chloride was allowed to react directly with the corresponding aniline (100-106) previously synthetized. The desired amides 93-99 were obtained in the 61-94% yield range. Moving forward, compounds 93-99 were converted to the desired targets 26, 31-36 by treatment with trifluoroacetic acid (TFA) in presence of thioanisole as scavenger. On the opposite of strategy applied in Scheme 1, the 4-methoxybenzyl protecting group was used, because it can be easily removed in acidic conditions, applicable also to molecules containing sulphur atoms and pyridine ring, both known to poison metal catalyst during hydrogenation in the last step.

The synthetic strategy used to produce compounds 25 and 38, were achieved via a diazotization of 2,3,5,6-tetrafluoro-[1,1′-biphenyl]-4-amine and commercial 4-phenoxyaniline using sodium nitrite and nitric acid and its was coupling with 108 under basic condition (Scheme 6). Compound 108 can be assimilated to a phenol, and the C-atom with the highest electron density is usually the preferred coupling position of diazonium salt. Due to the directing influence of hydroxyl groups in pyrazolo[1,5-a]pyridin-2-ol systems, coupling takes place at the 3-position.

The synthetic strategy used to produce compound 39 (Scheme 7) started from 2-hydroxypyrazolo[1,5-a]pyridine 44, that was protected with methyl iodine to afford the regioisomers 110a and 110b in ratio 77 and 13% respectively. The methyl protecting group can be easily removed in acidic conditions. The regioisomer 110a was allowed to react with hydrazine monohydrate to afford compound 111. The reactions proceeded through cyclo-condensation of 111 with 4-bromo-2,3,5,6-tetrafluorobenzoic acid to obtain compound 112. This latter was functionalized via Suzuki reaction to obtain compound 113. Moving forward, compound 113 was converted to the desired target 39 by treatment with BBr₃

For the synthesis of compounds 40-43 we designed a convergent synthetic approach with late-step Suzuki coupling of compound 114, as common intermediate. Also in this case the scheme started from 57, which was then used for the preparation of the common intermediate 114. Starting from acid 57, the corresponding acyl chloride was obtained via treatment with oxalyl chloride that was used without any further purification in the reaction with the lithium salt of 2,3,5,6-tetrafluoro-4-bromoaniline affording the desired amide 114 in the 80% yield. In this case, the transposition of the benzylic protecting group from the exocyclic oxygen to the endocyclic N1 nitrogen the pyrazolo[1,5-a]pyridine system was not observed and the yield was increased. Compound 57 was used as commune building block for the synthesis of desired compounds 119, 121 and 125 by Suzuki reaction involving the corresponding boronic acid pinacol ester (referrer Schemes 8, 9 and 10 for the synthesis) Compounds 119, 121 and 125 were then converted to the desired targets 40-42 by treatment with trifluoroacetic acid (TFA) in presence of thioanisole as scavenger. Finally compound 43 was obtained from compound 41 by reduction of ketone using NaBH 4.

hDHODH inhibitory activity and SAR. We evaluated the recombinant hDHODH inhibition activity of compounds 4-43 using three clinical-trial candidates (brequinar, BAY-2402234 and ASLAN003) and 1 as comparisons. While BAY-2402234 was purchased from a commercial source, brequinar was synthesized following known procedures. In order to complete the scenario and prepare the discussion of the following cell-based studies, LogD^{7.4}, solubility in PBS and protein binding were also measured for each compound.

SAR Analysis of the Pyridine Ring (A Ring) of the 2-Hydroxypyrazolo[1,5-a]Pyridine Scaffold.

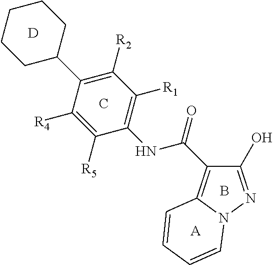

As reported in our previous publication,⁶ the interaction between 1 and the small lipophilic pocket created by Val134 and Val143 (subsite 4) was explored using Molecular Dynamics (MD) free energy perturbation (FEP),⁹ as a possible source of further modulation. Of the four sites on the A ring (positions 4-7), in-silico analyses suggested that position 7 is the most profitable for hydrogen substitution. Moreover, the study indicated that chlorine derivatives were generally preferred over methyl ones. Moving to experimental work (Table 1), taking into account the MD/FEP results, a derivative with a chlorine substituent in position 7 (4, IC₅₀=3.4 nM) was synthesized. Compared to the methyl analogue (2, IC₅₀=4.3 nM), the chlorine is better tolerated, leading to an analogue of 1 with comparable activity, but higher LogD^{7.4}. We therefore also considered a reduced 4,5,6,7-tetrahydropyrazolo[1,5-a]pyridine analogue, 5 (IC₅₀=5.8 nM), which gave a slight decrease in potency compared to 1. While the A ring modulations did not result in increased inhibitory activity compared to 1, higher LogDs^{7.4} were observed in all compounds, but were unfortunately all associated with reduced solubility. Solubility in PBS was not measured, as the concentration of the soluble fraction was below the LOD value (6 μM). In terms of protein binding, any significant improvement was observed.

TABLE 1
Enzymatic inhibitor activity of compounds 2-17, brequinar,
BAY-2402234, ASLAN003 and 1 as comparisons and relative
LogD^{7.4}, solubility and protein binding. The effect of the compounds
is expressed as IC₅₀ values. Limit of Detection (LOD):
value 6 μM. The “nd” notation indicates that
the compound was not tested in that specific assay.

| | hDHODH^{a} | | Solubility | Protein |
|---|---|---|---|---|
| | IC₅₀ ± SE | Log D^{7.4} ± | (μM) in | Binding |
| Compound | (nM) | SDc | PBS | (% bond) |
| Brequinar | 1.8 ± 0.3 | 1.83 ± 0.02 | 229 | 98.83 |
| BAY- | 6.0 ± 0.6 | 2.7¹⁰ | <1¹¹ | 90.1¹⁰ |
| 2402234 | (1.2 from lit¹⁰) | | | |
| ASLAN003 | 35¹² | nd | nd | >99¹² |
| 1 | 1.2 ± 0.2 | 2.35 ± 0.02 | 12 | 99.10 |
| 2 | 4.3 ± 0.5 | 2.70 ± 0.02 | <LOD | nd |
| 3 | 35 ± 3 | 2.47 ± 0.09 | <LOD | nd |
| 4 | 3.4 ± 0.5 | 2.81 ± 0.13 | <LOD | nd |
| 5 | 5.8 ± 0.4 | 2.36 ± 0.02 | <LOD | nd |
| 6 | 90.9 ± 13.1 | 0.66 ± 0.08 | 438 | nd |
| 7 | 1.35 ± 0.45 | nd | nd | nd |
| 8 | 6.23 ± 0.63 | 0.98 ± 0.03 | 47.3 | 99.58 |
| 9 | 150 ± 15 | 1.84 ± 0.06 | 20.2 | nd |
| 10 | 17.7 ± 3.30 | Insoluble | <LOD | nd |
| 11 | 2.03 ± 0.44 | 2.09 ± 0.04 | <LOD | 99.96 |
| 12 | 71.8 ± 9.42 | Insoluble | <LOD | nd |
| 13 | 6.34 ± 0.63 | 2.69 ± 0.03 | <LOD | 99.94 |
| 14 | 2.78 ± 0.32 | 1.82 ± 0.09 | 55.3 | nd |
| 15 | 2.30 ± 0.33 | 3.27 ± 0.19 | 8.1 | 100 |
| 16 | 2.75 ± 0.31 | 2.46 ± 0.04 | 74.3 | 99.95 |
| 17 | 4.09 ± 0.62 | 3.28 ± 0.12 | 12.9 | 100 |

SAR analysis of the D ring of the biphenylic scaffold: phenyl replacement/modulation. Moving to the D ring, we assigned the first four compounds (6-9) to the investigation of its possible isosteric replacement (Table 1). The incorporation of a morpholine substituent (6, IC₅₀=90.9 nM) was not well tolerated, as a phenyl ring, and resulted in around a 50-fold potency decrease compared to 1. The introduction of heteroatoms that may interact with the lipophilic sub-pocket, composed of Pro69 and Leu68, may induce repulsive interactions, as the potency decrease is also observed for pyridine derivate 9 (FIG. 1A). However, 6 was the most soluble of the series, showing almost twice the solubility of brequinar. The replacement of the D ring with classical bioisostere thiophen (7, IC₅₀=1.35 nM) retained the inhibitory profile. The optimal logD^{7.4} range for optimal drug absorption, via the phenomena of passive permeability or diffusion, is considered to be in the range between 1 and 3.¹³ In the case of hDHODH inhibitors, the literature indicates an optimal logD^{7.4} value superior to 2.50 reduce adsorption issue.¹¹ In terms of activity, the replacement of the phenyl position of 1 with a classic isostere nitrogen, as in 8 and 9, resulted in losses of activity (IC₅₀=6.23 nM and 150 nM), as meta replacement is better tolerated. To better understand this result, 9 must be compared with 13 (IC₅₀=6.34 nM) in which the —CF₃ in the meta position is still present, but the nitrogen is ideally removed. The two pyridine analogues, 8 and 9 display better solubility than 1, four- and 1.5-times respectively. In terms of protein binding, any significant improvement was observed.

Moving on, we investigated the positions on the D ring that are suitable for substitution in compounds 10-17. The binding mode of 1 and derivatives places the D ring next to the entrance of the ubiquinone binding pocket, exposing the meta position to an empty area of the binding site, on the border between the pocket and the vacuum. With 10-13, we investigated the effect of lipophilic substitutions, such as F and CF₃, in the para (10, 12) and meta (11, 13) positions of the D ring. Analyzing the results (Table 1), it can be observed how meta replacement, 11 and 13 with IC₅₀=2.03 nM and IC₅₀=6.34 nM respectively, were better tolerated than the para isomers, 10 and 12 with IC₅₀=17.7 nM and IC₅₀=71.8 nM. While finding the activity of 11 to be in the same range as 1 is not surprising, as the fluorine is a classical proton bioisosteric replacement, this cannot be said for 13, for which small lipophilic groups, such as —CF₃, are well accepted. This replacement validated the predicted binding mode of 13, in which a trifluoromethyl is placed in an empty area of the binding site. These modulations resulted in compounds being more lipophilic, as expected, but unfortunately, this property is associated with insolubility, and these values are largely below the reference limit of 6 μM. Focusing on substitution on the meta position, we obtained compound 14 (IC₅₀=2.78 nM), which is comparable to 1 itself in terms of potency, but characterized by better solubility (around five times), as the oxygen atom is able to form hydrogen bonds with water, and has a logD^{7.4} comparable with that of brequinar itself. By ideally modulating 14, we introduced substitution to the phenolic oxygen, giving 15-17 (Table 1) and 40-43 (Table 2). This modulation resulted in an IC₅₀ that is comparable to that of 1, proving the predicted binding mode again, and was associated with an increase in LogD^{7.4} for each compound. The most interesting compounds are 17, 40-43 which are characterized by the introduction of a propoxy group. These are the most interesting compounds in the series here described, as they are comparable to the lead 1 in terms of potency, although the compound 17 showing similar solubility but higher LogD^{7.4} (above the 2.5 threshold).

SAR analysis of the biphenylic scaffold. Although its potent inhibition of hDHODH evaluated in vitro, compound 1 solubility profile is far away from that of brequinar and the presence of the tetrafluorobiphenylic substituent is detrimental for its solubility. With the purpose to obtain an alternative scaffold that will provide hDHODH inhibition comparable to that of compound 1, a new series of inhibitors was developed starting from lead structure of compound 18⁶. Eighteen analogues (compound 19-24, 26-37) that lacked the biphenylic scaffold were therefore deigned to explore novel possibilities. (FIGS. 2A and 2B) The inspiration was taken from Das et al. 14 in which “brequinar-like” activity was replicated by analogues which included a substituted diarylether. The rationale behind the design of targets 26 rises from isosteres concept. Since compound 18 showed good interactions within the enzyme binding pocket, the general structure of the molecule was maintained, but to achieve a more polar compound, it has been decided to develop a target where ring D has been replaced by a more polar isostere: the pyridine ring with the oxygen bridge between ring C and D changed into a sulphur one. Targets 27-35 represent the proposing compounds after SAR studies on compound 18 in attempt to increase its binding affinity. The presence of additional ether substituent could explore in the binding site a region that has still remained almost unexploited. The investigation on the possibility of additional hydrophobic interactions with some lipophilic amino acidic residues located in subsite 1 conduced to the development of differently substituted diaryl ethers which might make the compounds gain in potency and selectivity for hDHODH. Table 2 shows the result of the SAR study directed to modulate 18, which differs from 1 for the presence of an aryloxy moiety demanded to interact with hDHODH subsite 1.

The replacement of phenoxyphenyl moiety by the azotated moieties indole-5-yl, N-phenylindole-5-yl, N1-phenylbenzominidazol-5-yl or N1-phenylbenzotriazolyl (compounds 19-22 respectively) is detrimental for the activity as well as the replacement of one of the two phenyl by a pyridine ring (compound 23, 24, 26). The removal of the methyl in orto position respect to the amide function (compound 27) decrease ten time the activity while the removal of the methyl in meta position (compound 28) is detrimental highlighting the importance of the small substituent in the meta position. In fact, the substitution of the methyl substituent in meta position by bigger chain as isopropyl (compound 30) halve the activity. Moving to the orto position, huge substituents as isopropyl (compound 29) increase the activity, while its replacement by different alkyloxy moieties cancel the activity (compounds 31-35). Also in the phenoxyphenyl frame, a trifluoromethyl substituent in para position (compound 37) decrease the activity.

SAR analysis of the “M-Q” and “Het” linkers. The replacement of the amide function by the diazo function maintains the activity only in the presence of the tetrafluorobiphenylic substituent as showed by the IC50 of derivative 25 and 38 showing that the bioisosteric properties between amide and diazo linkers are present only in compound 25. While the replacement of the amide function by 1,3,5-oxadiazole moieties is not beneficial for the activity as showed by the very low IC50 of derivative 39 in comparison with compound 1.

TABLE 2
Enzymatic inhibitor activity of compounds 18-43, and relative
LogD^{7.4} and solubility. The effect of the compounds is expressed
as IC₅₀ value. Limit of Detection (LOD): value 6 μM.

| | hDHODH^{a} | Log | Solubility |
|---|---|---|---|
| Compound | IC₅₀ ± SE (nM) | D^{7.4} ± SDc | (μM) in PBS |
| 18⁶ | 43 ± 5 | nd | 1.72 ± 0.03 |
| 19 | >10000 | 0.45 ± 0.02 | nd |
| 20 | 2757 ± 386 | 2.89 ± 0.05 | nd |
| 21 | >10000 | 2.55 ± 0.04 | nd |
| 22 | 7196 ± 1600 | 1.59 ± 0.03 | nd |
| 23 | 12000 ± 1000 | nd | nd |
| 24 | >10000 | nd | nd |
| 25 | 13 (n = 2) | nd | |
| 26 | 2209 ± 366 | 1.76 ± 0.07 | nd |
| 27⁶ | 480 ± 31 | nd | nd |
| 28 | 400 ± 60 | 2.46 ± 0.05 | nd |
| 29 | 7.2 ± 0.9 | 3.18 ± 0.09 | 1.2 ± 0.28 |
| 30 | 114 ± 11 | 2.91 ± 0.07 | 0.98 ± 0.23 |
| 31 | 5201 ± 924 | nd | nd |
| 32 | >10000 | nd | nd |
| 33 | 3938 ± 797 | nd | nd |
| 34 | 2209 ± 346 | nd | nd |
| 35 | 6983 ± 1007 | nd | nd |
| 36 | 70 ± 11 | 2.51 ± 0.07 | 111.1 ± 0.35 |
| 37 | 18 ± 4 | >3 | 1.23 ± 0.09 |
| 38 | Inactive at 1 microM | nd | nd |
| 39 | Inactive at 1 microM | nd | nd |
| 40 | 1.48 ± 0.20 | nd | nd |
| 41 | 0.82 ± 0.10 | nd | nd |
| 42 | 3.29 ± 0.33 | nd | nd |
| 43 | 2.50 ± 0.20 | nd | nd |

Physicochemical Properties

The determination of the main physicochemical properties that govern the ADME profile was carried out for all compounds by measuring their lipophilicity (logD7.4) and solubility at physiological pH.

Thermodynamic solubility was determinate using the equilibrium shake flask thermodynamic solubility method in phosphate saline buffer (PBS at pH=7.4), chosen for its capability to simulate plasmatic pH and osmolarity.

Lipophilicity. The shake flask method was chosen to determinate logD7.4. Besides, lipophilicity was estimated in silico, clogP has been calculated for each compound with the software Bio-Loom for Windows, version 1.5.

Summarizing the results, an optimization in terms of solubility was observed for compound 6 (438 μM), due to the depletion of the second aromatic ring and the insertion of a morpholine ring. This replacement gives to the molecule two important polar contributions: an amine and an ether. Also compounds 8 and 14 represent an effort to increase solubility, introducing a pyridine and a phenol ring instead of ring C. These compounds allow to move from 12 μM of the analogue compound 1 to 47 μM and 55 μM. Target 17, with its propoxy chain, has almost the same low solubility (12.9 μM) of compound 1, so it does not represent an effective improvement in this sense.

In general it is possible to say that all the measured solubility are far away from that of brequinar and this low solubility has a reasonable explication: at pH=7.4, the hydroxy moiety of most compounds is completely deprotonated (pKa˜5-6) and it has been hypothesized that an intramolecular reinforced hydrogen bond takes place between the amidic hydrogen and the deprotonated hydroxy group, consequently forming a stable six-term ring.

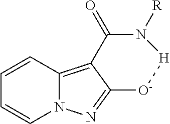

In this way, the amidic hydrogen would not be available to hydrogen-binding (HBD) to water's molecules and thus is not contributing for solubility. This hypothesis is confirmed by the crystallographic pose of that molecule in the enzyme, where the intramolecular HB is highlighted.

All the compounds have a good lipophilic-hydrophilic balance with optimal logD values for a favourable pharmacokinetic behaviour; the differences observed between clogP and measured logD7.4 agree with the large ionization of these compounds at physiological pH.

The goal for lipophilicity is a value between 1 and 3, that is an optimal value for absorption by passive diffusion permeability after oral dosing. Compounds 6, 8 and 14 that are characterised by a morpholine, a pyridine and a phenol ring, have a very low lipophilicity, instead their solubilities are high; as result these molecules probably are not able to pass membranes. Compound 17 presents a propoxy substituent that could contribute to make it able to easily cross phospholipids bilayers. Even though this target seems to have a good attitude to reach the enzymatic target, it is very insoluble and therefore difficult to use for in vitro tests. A good compromise appears to be compound 26 in which the presence of the pyridine allows to have a better solubility and, at the same time, the thioether a good lipophilic-hydrophilic balance.

Antiviral Profile of Selected Compounds.

Compound 1 as a new potent BSAA candidate. To investigate the feasibility of targeting hDHODH activity to develop pan-coronavirus HTA, 13 newly designed hDHODH inhibitors (Table 3) were selected for antiviral activity against the prototypical human β-cov, hCoV-0C43, by using FFRAs in which test compounds were present before, during and after infection (full-treatment). As shown in Table 3, when tested at 0.1 μM, six of hDHODH inhibitors (1, 9, 16, 17, 40) were able to decrease hCoV-0C43 replication more than 50%. Notably, compound 1, MEDS433, was the most effective among the tested hDHODH inhibitors, since it abrogated completely hCoV-0C43 replication. Compound 1 was therefore selected for further investigations. Having such an interesting preclinical hDHODH inhibitor in hand, we assayed 1 for antiviral activity against a large panel of human viruses.

TABLE 3
% Decrease of hCoV-OC43 replication in presence of inhibitor
0.1 μM. HCT8 cells were pretreated and treated vehicle (DMSO)
or with 0.1 μM of the different hDHODH inhibitors during
infection with hCoV-OC43 (100 PFU/well), and throughout the
experiment. At 72 h p.i., viral foci were immunostained and
the mean foci number in treated culture compared to that of
DMSO-treated and hCoV-OC43-infected control HCT8 cell monolayers.

| | % Decrease of hCoV-OC43 |
|---|---|
| | replication in presence of |
| Compound | inhibitor 0.1 μM |
| 1 | 99.30 |
| 5 | 31.30 |
| 9 | 52.56 |
| 15 | 93.59 |
| 16 | 94.87 |
| 17 | 85.89 |
| 25 | 18.70 |
| 37 | 36.09 |
| 40 | 97.39 |

Herpes simplex virus. Incidence and severity of HSV infections have increased over the past decades due to the increasing number of immunocompromised patients, with genital herpes infection becoming one of the world's most prevalent sexually transmitted infections (STIs). In the absence of efficient preventive vaccines, the control of HSV infections, in particular genital herpes, thus remains a high-priority. These facts highlight the need to development of new anti-HSV agents, possibly endowed with a mechanism of action different from that of approved nucleoside analogs (acyclovir, famcyclovir, valacyclovir) whose prolonged use may lead to treatment failures due to the development of antiviral-resistant strains. To this end, we tested 1 against HSV-1 and HSV-2 compared to brequinar. As shown in Table 4, 1 potently inhibited HSV-1 and HSV-2 replication (PRA, Vero cells) with EC₅₀ of 0.110 and 0.170 μM, respectively. Noteworthy, the anti-HSV activity of 1 was about one order of magnitude more potent than that of brequinar, and even lower than that of the reference drug ACV (0.180 μM). Further preliminary observations indicated that the anti-HSV activity of 1 was associated to an inhibition of viral DNA synthesis and Late protein expression, while Immediate-Early proteins were not significantly reduced, thus indicating that 1 targets a phase of HSV replication cycle prior to viral DNA replication, in agreement to its ability to inhibit hDHODH activity, thus causing pyrimidine depletion.

TABLE 4
Activity against the replication of different viruses of
compound 1 and 17, compared to brequinar, remdesivir and
EIDD-1931, the drug released by pro-drug molnupiravir.

| Compound | Virus | EC₅₀^{a} (μM) | CC₅₀^{b} (μM) | SI^{c} |
|---|---|---|---|---|
| 1 | SARS-CoV-2 | 0.063 | >500 | >7,900 |
| | hCoV-OC43 | 0.012 | 78.48 | >6,329 |
| | a-hCoV-229E | 0.022 | 104.8 | >4,763 |
| | HSV-1 | 0.110 | 234 | 2,127 |
| | HSV-2 | 0.170 | 234 | 1,376 |
| | RSV | 0.008 | 84 | >10,000 |
| | Influenza A | 0.120 | 12.2 | 102 |
| 17 | SARS-CoV-2 | Nd | nd | nd |
| | HSV-1 | 0.090 | 117 | 1300 |
| | HSV-2 | 0.110 | 117 | 1063 |
| | RSV | Nd | nd | nd |
| | Influenza A | Nd | nd | nd |
| Brequinar | SARS-CoV-2 | 0.200 | — | — |
| | hCoV-OC43 | 0.0427 | | |
| | a-hCoV-229E | 0.022 | | |
| | HSV-1 | 0.53 | 100 | 188 |
| | HSV-2 | 1.77 | 100 | 56 |
| | RSV | 0.015 | 168 | 11,200 |
| | Influenza A | 0.76 | 5.0 | 6.6 |
| Remdesivir | SARS-CoV-2¹⁵ | 0.77 | >100 | >129.87 |
| | hCoV-OC43 | 0.147 | | |
| | a-hCoV-229E | 0.0348 | | |
| EIDD-1931 | SARS-CoV-2¹⁶ | 0.3 | >10 | 33 |
| | RSV¹⁷ | 0.69 | | 394 |

Respiratory viruses: Respiratory viral infections (RVI) are a global health concern in terms of morbidity and mortality. Influenza virus, respiratory syncytial virus and coronavirus are among the most common viruses causing lower respiratory tract infections.

Influenza virus. Influenza viruses A and B are widespread major human pathogens and responsible for seasonal epidemics and pandemics. Seasonal vaccines represent the most effective measure to prevent and control Influenza infections. Treatment of Influenza infections may also benefit from two classes of licensed DAA drugs, such as matrix protein inhibitors and neuraminidase inhibitors. However, their use is severely restricted by selection of resistance strains. Thus, the development of alternative anti-Influenza compounds, both effective against antigenically different viruses and characterized by new mechanisms of action, is an urgent priority. 1 was therefore tested against a reference strains of Influenza A virus (A/Puerto Rico/8/34) and it showed a potent inhibitory activity (EC₅₀ 0.120 μM, PRA, MDCK cells, Table 4). Again, 1 performed better than brequinar with an EC₅₀ value of more than 6 times lower.

Respiratory syncytial virus. Respiratory syncytial virus (RSV) is the most important cause of lower-respiratory tract infections in infants and young children, leading to severe bronchiolitis and pneumonia. Nevertheless, no vaccines are yet available and antiviral treatment are restricted to palivizumab for preventive treatment and ribavirin, a nucleoside purine analog, which is penalized by severe drawbacks. Thus, there is an urgent medical need to develop new compounds able to block RSV replication. With this in mind, we tested 1 vs RSV and a very potent antiviral activity (EC₅₀ 0.008 μM) (Table 3) was measured in HEp-2 cells associated with an effective SI (>10,000, CC₅₀ 84 μM), and with a profile slightly superior to that of brequinar (EC₅₀ 0.015 μM). Moreover, to verify whether the observed inhibition of RSV replication by 1 resulted from the blockage of pyrimidine biosynthesis, antiviral assays were repeated using uridine-supplemented culture medium. Since uridine can be converted to UMP via the salvage pathway (FIG. 3), cells can bypass the requirement for the de novo pyrimidine biosynthesis. As shown in FIG. 3, the uridine supplement abrogated the anti-RSV of 1, thus confirming that the pyrimidine pathway is affected by 1 in RSV-infected cells. However, UMP from which all the other pyrimidines are derived, can be generated by both the salvage pathway and de novo biosynthesis. To distinguish between the two pathways, we examined, the effects of addition of the substrate (Dihydroorotic acid, DHO) or product (Orotic acid, ORO) of hDHODH for their ability to restore RSV replication. Consistent with a pivotal role of hDHODH inhibition in the anti-RSV activity of 1, the addition of ORO but not DHO reversed the inhibitory effect of 1 (FIG. 3). Taken together, these preliminary observations suggest the specific targeting of hDHODH activity by 1 as the mechanism of the antiviral activity against RSV.

α- and β-coronavirus. Effective pan-CoVs antivirals can be highly valuable to fill the gap in the control of emerging CoVs diseases given their rapid repositioning from one pandemic event to another. A significant concentration-dependent inhibition of hCoV-0C43 replication was then confirmed in HCT-8 cells treated with compound 1 (Table 4). The measured EC₅₀ was 0.0124±0.0034 μM. Compound 1 was also very effective against another hCoV, the prototypic β-hCoV-229E, whose replication in MRC5 fibroblasts was severely impaired (Table 4) with EC₅₀ value of 0.022±0.003 μM. The comparison with the reference drug RDV, used as a positive control for anti-hCoV antiviral activity, highlighted an anti-hCoV-229E potency of compound 1 comparable to that of RDV (EC₅₀ 0.0348±0.005 μM), while the hDHODH inhibitor was much more effective than RDV (EC₅₀ 0.147±0.034 μM) against hCoV-0C43. To the contrary, compound 1 was more effective than brequinar against hCOV-229E, as the EC₅₀ of the latter was 0.0427±0.003 μM, whereas against hCoV-0C43 the EC₅₀ of brequinar (0.022±0.003 μM) was commensurate with that of compound 1. Finally, the anti-hCoVs activity of compound 1 was not due to cytotoxicity of target cells themselves, since its cytotoxic concentration (CC₅₀) as determined in uninfected cells was 78.48±4.6 μM for HCT8 cells, and 104.80±19.75 μM for MRC5 fibroblasts, with a favorable Selective Index (SI) greater than 6,329 and 4,763 for hCoV-0C43 and hCoV-299E, respectively.

SARS-CoV-2. The emergence of COVID-19 as global public health emergency in the past few months prompted us to immediately investigate the sensitivity of SARS-CoV-2 to 1. As shown in Table 4 and FIG. 4, we measured a potent antiviral effect (EC₅₀ 0.063 μM) (PRA, Vero E6 cells) against the COVID-19 virus that was associated to a very safe profile (SI>7900, CC₅₀>500 μM). Just to allocate these numbers, the anti-SARS-CoV-2 activity of 1 is one order of magnitude more potent than that of the Best Available Therapy Remdesivir (EC₅₀ 0.77 μM) and five time more potent than EIDD-1931, the drug released by pro-drug Molnupiravir.

Taken as a whole, these preliminary results sustained by a strong rational on the development of hDHODH inhibitors as BSAAs, strongly validate the proposal to further develop 1 as an effective anti-SARS-CoV-2 agent and a potent and safe BSAA that could be very valuable also in future pandemic scenarios.

Conclusions

The present inventors identified a novel class of inhibitors that are based on hydroxyl-pyrazolo[1,5-a]pyridine, an unusual bioisostere of the carboxylic acid function. Compounds 1 and 17, the most powerful hDHODH inhibitors yet discovered, show brequinar-like hDHODH potency levels in vitro and are superior in terms of antiviral potency and selectivity blocking the viral replication at concentrations that are one log digit lower than those achieved in experiments with brequinar.

Since compounds 1 and 17 target a cellular gene product (DHODH), and it is unlikely that viruses can overcome the need for pyrimidine nucleotides, the likelihood that their therapeutic effect will be compromised by the development of viral resistance is low. This may be particularly important for RNA viruses whose high mutation frequency often promotes evasion of direct-acting antivirals.

The key to a successful COVID-19 treatment is to not only have a potent molecule, but to also have a dose that can be delivered safely and that will sustain exposure in the blood to inhibit viral replication or infection.

It is therefore apparent that compound 1 displays an optimal toxicity profile and highly selective on-target activity, making it an ideal candidate for further in vivo studies in SARS-CoV-2 models. Against other virus, compound 1 was found also effective being its EC₅₀ always below 170 nM

Materials and Methods

Chemistry

General methods. All chemical reagents were obtained from commercial sources (Sigma Aldrich, Alfa Aesar, FluoroChem), and used without further purification. Thin-layer chromatography (TLC), was carried out to monitor reaction progress. Analytical grade solvents (acetonitrile, diisopropyl ether, diethyl ether, dichloromethane [DCM], dimethylformamide [DMF], ethanol 99.8% v/v, ethyl acetate [EtOAc], hexane, methanol [MeOH], petroleum ether b.p. 40-60° C. [petroleum ether], toluene), were used without further purification. When needed, solvents were dried over 4 Å molecular sieves. Tetrahydrofuran (THF), was distilled from Na and benzophenone under N₂ immediately prior to use. Thin layer chromatography (TLC), on silica gel was carried out on 5×20 cm plates at 0.25 mm layer thickness. Anhydrous Na₂SO₄ was used as a drying agent for the organic phases. Compound purification was either achieved using flash column chromatography on silica gel (Merck Kieselgel 60, 230-400 mesh ASTM), and the eluents indicated in the procedures for each compound, or using CombiFlash Rf 200 (Teledyne Isco), with 5-200 mL/min, 200 psi (with automatic injection valve), and RediSep Rf Silica columns (Teledyne Isco), with the eluents indicated in the procedures for each compound. Compounds synthesized in our laboratory generally varied between 90% and 99% purity. Biological experiments were performed on compounds with a purity of at least 95%. Purity was checked using two UHPLC analytical methods. HPLC analyses were performed on an UHPLC chromatographic system (Perkin Elmer, Flexar). The analytical column was an UHPLC Acquity CSH Fluoro-Phenyl (2.1×100 mm, 1.7 μm particle size, Waters) and reverse-phase (RP) C18 Phenomenex column (2.1×100 mm, 1.7 μm particle size). Compounds were dissolved in acetonitrile and injected through a 20 μl loop. The mobile phase consisted of acetonitrile/water with 0.1% trifluoroacetic acid (ratio between 60/40 and 40/60, depending on the compound's retention factor). UHPLC retention times were obtained at flow rates of 0.5 mL/min, and the column effluent was monitored at 254 and 262 nm, referenced against a 360 nm wavelength. Melting points (m.p.), were measured on a capillary apparatus (Büchi 540). Final m.p. determination was achieved by placing the sample at a temperature 10° C. below the m.p. and applying a heating rate of 1° C. min⁻¹. All compounds were routinely checked by ¹H- and ¹³C-NMR and mass spectrometry. The IR spectra of solid compounds were recorded on FT-IR (PerkinElmer SPECTRUM BXII, KBr dispersions), using the diffuse reflectance apparatus DRIFT ACCY. MS spectra were performed on a Waters Micromass ZQ equipped with an ESCi source for electrospray ionization mass spectra. ¹H- and ¹³C-NMR spectra were performed on a JEOL ECZR600. The following abbreviations are used for coupling patterns: br=broad, s=singlet, d=doublet, dd=doublet of doublets, t=triplet, q=quartet, m=multiplet. Chemical shifts (5) are given in parts per million (ppm). In this work protons and carbons are labelled (a, b, c, d, e, f, g, h, 1, m, n, o, p, q, r and s) according to figure in supporting info. Values marked with an asterisk (*, ** and ***) are interchangeable. Detailed ¹³C spectra of tetrafluorinated biphenyl compounds (final compounds 4-17 and protected final compounds) have not been entirely reported due to their especially complicated patterns (attributable to the multiple couplings between fluorine and carbon atoms). For these spectra, only the ¹³C signals caused by the heterocyclic substructure and non-aromatic carbons are assigned. For the final compounds 4-17, HRMS spectra were recorded on an LTQ-Orbitrap XL Plus (Thermo Scientific, Bremen, Germany) mass spectrometer, equipped with an atmospheric pressure interface and an ESI ion source instrument. Compounds 44, 45^{6, 18} has been prepared according to previously-described.

Ethyl 2-((4-methoxybenzyl)oxy)pyrazolo[1,5-a]pyridine-3-carboxylate (56a) and ethyl N-(4-methoxybenzyl)-2-oxo-pyrazolo[1,5-a]pyridine-3-carboxylate (56b). 4-Methoxybenzyl bromide (645 mg, 3.20 mmol, 1.10 eq) was added dropwise to a mixture of 44 (600 mg, 2.91 mmol) and Cs₂CO₃ (2.295 g, 7.04 mmol, 2.4 eq) in dry DMF (15 mL). The reaction mixture was stirred overnight at room temperature, then water (100 mL) was added. The mixture was extracted with EtOAc (4×70 mL), the combined organic layer was dried under Na₂SO₄ and evaporated under reduced pressure to afford a yellow oil. This latter showed two spots on TLC (eluent: petroleum ether/EtOAc 60/40 v/v), ascribed to the two pyrazolo[1,5-a]-pyridine regioisomers. The mixture was separated using flash chromatography (eluent: petroleum ether/EtOAc 2/1 v/v, then dichloromethane/MeOH 95/5 v/v).

56a) First isomer eluted. White solid after trituration with hexane followed by water (111.3-112.5° C.). Yield 61%. ¹H NMR (600 MHz, Chloroform-d) δ 1.40 (t, J=7.1 Hz, 3H, —OCH₂CH₃); 3.81 (s, 3H, —OCH₃), 4.36 (q, J=7.1 Hz, 2H, —OCH₂CH₃), 5.43 (s, 2H, —OCH₂Ar), 6.83 (t, 1H, J=6.7 Hz, H-b), 6.91 (d, 2H, J=8.6 Hz, H-n), 7.35 (t, 1H, J=7.7 Hz, H-c), 7.48 (d, 2H, J=8.5 Hz, H-m), 8.00 (d, 1H, J=8.8 Hz, H-d), 8.29 (d, 1H, J=6.8 Hz, H-a), ¹³C NMR (151 MHz, Chloroform-d) δ 14.7 (—OCH₂CH₃), 55.4 (—OCH₃), 59.7 (—OCH₂CH₃), 70.7 (—OCH₂Ar), 88.5 (C-f), 112.6 (C-b), 113.9 (C-n), 118.3 (C-d), 127.8 (C-c)*, 128.9 (C-a)*, 129.0 (C-l)*, 129.3 (C-m), 142.9 (C-e), 159.5 (C-o), 163.4 (C-g)*, 165.2 (C-h)*. MS (ES+): 327 (M+1).

56b) Second isomer eluted. White solid. (158.3-159.2° C., from diisopropyl ether). Yield 27%. ¹H NMR (600 MHz, DMSO-d₆) δ 1.28 (t, 3H, J=7.1 Hz, —OCH₂CH₃); 3.69 (s, 3H, —OCH₃), 4.21 (q, 2H, J=7.1 Hz, —OCH₂CH₃), 5.35 (s, 2H, —NCH₂Ar), 6.88 (d, 2H, J=8.5 Hz, H-n), 6.96 (t, 1H, J=6.8 Hz, H-b), 7.19 (d, 2H, J=8.4 Hz, H-m), 7.58 (t, 1H, J=8.0 Hz, H-c), 7.91 (d, 1H, J=8.8 Hz, H-d), 8.45 (d, 1H, J=6.8 Hz, H-a); ¹³C NMR (151 MHz, DMSO-d₆) δ 14.6 (—OCH₂CH₃), 43.2 (—NCH₂Ar), 55.1 (—OCH₃), 58.5 (—OCH₂CH₃). 83.5 (C-f), 112.4 (C-b), 114.3 (C-n), 116.3 (C-d), 125.3 (C-a), 125.7 (C-l), 128.8 (C-m), 132.4 (C-c), 142.8 (C-e), 159.0 (C-o), 160.0 (C-h)*, 163.2 (C-g)*; MS (ES+): 327 (M+1).

2-((4-Methoxybenzyl)oxy)pyrazolo[1,5-a]pyridine-3-carboxylic acid (57). 6 M NaOH (5.0 eq) was added to a solution of compound 56a (785 mg, 2.40 mmol) in EtOH (20 mL). The mixture was stirred for 4 h at 75° C., then neutralized with 6 M HCl and concentrated under reduced pressure. The mixture was cooled 0° C. then acidified with 2 M HCl until pH 2 was reached, obtaining a suspension. This latter was filtered to get 57 as a white solid (162.8-163.9° C. dec., from water). Yield 90%. ¹H NMR (600 MHz, DMSO-d₆) δ 3.76 (s, 3H, —OCH₃), 5.34 (s, 2H, —OCH₂Ar), 6.96 (d, 2H, J=8.4 Hz, H-n), 7.02 (t, 1H, J=6.7 Hz, H-b), 7.45 (d, 2H, J=8.3 Hz, H-m), 7.51 (t, 1H, J=7.9 Hz, H-c), 7.92 (d, 1H, J=8.8 Hz, H-d), 8.66 (d, 1H, J=6.7 Hz, H-a), 12.07 (s, 1H, —COOH); ¹³C NMR (151 MHz, DMSO-d₆) δ 55.1 (—OCH₃), 70.1 (—OCH₂Ar), 87.6 (C-f), 113.1 (C-b), 113.8 (C-n), 117.3 (C-d), 128.4 (C-a)*, 128.5 (C-l)*, 129.5 (C-c)*, 129.9 (C-m), 142.3 (C-e), 159.2 (C-o), 163.5 (C-h)*, 164.4 (C-g)*; MS (ES+): 299 (M+1).

N-(4-Bromo-2,3,5,6-tetrafluorophenyl)-2-((4-methoxybenzyl)oxy)pyrazolo[1,5-a]pyridine-3-carboxamide (58). Oxalyl chloride (0.54 mL, 6.30 mmol, 3.0 eq) and dry DMF (1 drop) were added to a cooled (0° C.) solution of 57 (630 mg, 2.10 mmol) in dry THF (15 mL) kept under a nitrogen atmosphere; the resulting mixture was stirred for 2 h at room temperature. In parallel a 2M solution of AlMe₃ in toluene (1.8 mL, 3.57 mmol, 1.7 eq) was added to a solution of 4-bromo-2,3,5,6-tetrafluoroaniline (769 mg, 3.15 mmol, 1.5 eq) in dry toluene (10 mL) under nitrogen atmosphere. The resulting suspension was stirred 3 h at room temperature. The solution of acyl chloride was then concentrated under reduced pressure, and the residue was dissolved in dry THF (10 mL, this step was repeated three times). The acyl chloride was dissolved in dry toluene (15 mL) and the solution was added to the above described suspension. The reaction mixture was stirred at 85° C. overnight, then cooled to room temperature, quenched with methanol, then evaporated. The residue was dissolved in EtOAc (80 mL), 0.5 M HCl (50 mL) was then added and layers resolved. The combined organic layers were washed with brine, dried and evaporated under reduced pressure. The crude material was purified using flash chromatography (eluent: petroleum ether/EtOAc/DCM 2/1/1 v/v/v) to afford the title compound as white solid (177.4-178.0° C., triturated with diisopropyl ether). Yield 55%. ¹H NMR (600 MHz, Chloroform-d) δ 3.79 (s, 3H, —OCH₃), 5.41 (s, 2H, —NCH₂Ar), 6.77 (t, 1H, J=6.9 Hz, H-b), 6.90 (d, 2H, J=8.5 Hz, H-n), 7.21 (d, 2H, J=8.5 Hz, H-m), 7.46 (t, 1H, J=7.9, Hz, H-c), 7.75 (d, 1H, J=6.9 Hz, H-a), 8.27 (t, 1H, J=8.8 Hz, H-d), 9.98 (s, 1H, —NH); n C NMR (151 MHz, Chloroform-d) δ 45.2 (—NCH₂Ar), (—OCH₃), 87.1 (C-f), 96.4 (t, J=22.6 Hz, C-s)*, 112.9 (C-b), 115.0 (C-n), 117.0 (t, J=14.8 Hz, C-p)*, 118.3 (C-d), 123.1 (C-a), 124.2 (C-l), 128.6 (C-m), 131.8 (C-c), 142.5 (C-e), 142.8 (dd, J=251.6, 14.9 Hz, (C-r)**, 145.2 (dd, J=246.4, 14.2 Hz, (C-q)**, 160.0 (C-o)***, 161.4 (C-h)***, 162.2 (C-g)***; MS (ES+): 524/526 (M+1).

1-(4-Methoxybenzyl)-2-oxo-N-(2,3,5,6-tetrafluoro-4-morpholinophenyl)-1,2-dihydropyrazolo[1,5-a]pyridine-3-carboxamide (59). To a solution of 58 (420 mg, 0.80 mmol, 1.00 eq) and morpholine (209 mg, 2.40 mmol, 3.00 eq) in toluene (30 mL) was added Cs₂CO₃ (782 mg, 2.4 mmol, 3.00 eq). After degasification with nitrogen for 10 min, Pd(OAc)₂ (18 mg, 0.08 mmol, 0.10 eq) and BINAP (100 mg, mmol, 0.20 eq) were added, and mixture was degassed again for 5 min. The resulting suspension was heated at 110° C. in sealed flask under atmosphere of nitrogen. After 3.5 h, the heating was stopped, the mixture concentrated to reduced pressure then water was added. The resulting suspension was extracted with EtOAc (3×50 mL), the combined organic fractions collected, dried and concentrated under reduced pressure. The crude was purified by flash chromatography (eluent: petroleum ether/EtOAc/DCM 1/1/1 v/v/v) affording a solid crude that was triturated with diisopropyl ether to obtain the title compound as a white solid (237.2-237.5° C. dec). Yield: 59%. ¹H NMR (600 MHz, Chloroform-d) δ 3.24-3.28 (m, 4H, —NCH₂CH₂O—), 3.79 (s, 3H, —OCH₃), 3.81-3.85 (m, 4H, —NCH₂CH₂O—), 5.40 (s, 2H, —NCH₂Ar), 6.74 (t, 1H, J=6.9 Hz, H-b), 6.90 (d, 2H, J=8.5 Hz, H-n), 7.21 (d, 2H, J=8.5 Hz, H-m), 7.44 (t, 1H, J=7.9 Hz, H-c), 7.73 (d, 1H, J=6.9, Hz, H-a), 8.28 (d, 1H, J=8.9 Hz, H-d), 9.75 (s, 1H, —NH); ¹³C NMR (151 MHz, Chloroform-d) δ 45.1 (—NCH₂Ar), 51.5 (—NCH₂CH₂O—), 55.5 (—OCH₃), 67.5 (—NCH₂ CH₂O—), 87.2 (C-f), 111.3 (t, J=15.5 Hz, C-p)*, 112.6 (C-b), 114.9 (C-n), 118.3 (C-d), 123.1 (C-a), 124.3 (C-l), 127.8 (t, J=11.0 Hz, C-s), 128.6 (C-m), 131.6 (C-c), 142.5 (C-e), 143.2 (d, J=248.2 Hz, C-q)**, 143.5 (d, J=247.9, Hz, C-r)**, 160.0 (C-o)***, 162.0 (C-h)***, 162.1 (C-g)***; MS (ES+): 553 (M+Na).

General Procedure: the Suzuki reaction used for production of compounds 60-66. Pd(PPh₃)₄ (90 mg, 0.08 mmol, 0.20 eq) was added to a solution of 58 (200 mg, 0.38 mmol, 1.00 eq) and K₂CO₃ (158 mg, 1.14 mmol, 3.00 eq) in dioxane/water mixture (9:1 v/v). After stirring the resulting mixture under atmosphere of nitrogen for 1 h at r.t. the corresponding boronic acid (0.760 mmol, 2.0 eq) was added; the reaction mixture was then heated at reflux under atmosphere of nitrogen. After 2 h, an additional amount of boronic acid (0.38 mmol, 1.0 eq) was added, the reaction mixture was heated at reflux for another 2 h before cooling to room temperature and concentrating under reduced pressure. The crude material was taken-up with water (100 mL) and the mixture was extracted with EtOAc (3×60 mL), the combined organic layers were dried over Na₂SO₄ and concentrated under reduced pressure. The crude was purified by flash chromatography (see below the conditions).

1-(4-Methoxybenzyl)-2-oxo-N-(2,3,5,6-tetrafluoro-4-(thiophen-2-yl)phenyl)-1,2-dihydropyrazolo[1,5-a]pyridine-3-carboxamide (60). The crude was purified by flash chromatography (eluent: petroleum ether/EtOAc 1/1 v/v) obtaining a solid crude that was recrystallized from acetonitrile (8 mL) to obtain the title compound as white solid (197.4-198.1° C. from acetonitrile). Yield: 72%. ¹H NMR (600 MHz, Chloroform-d) δ 3.79 (s, 3H, —OCH₃), 5.41 (s, 2H, —NCH₂Ar), 6.76 (t, 1H, J=6.6 Hz, H-b), 6.90 (d, 2H, J=8.5 Hz, H-n), 7.16-7.20 (m, 1H, aromatic proton), 7.21 (d, 2H, J=8.5 Hz, H-m), 7.45 (t, 1H, J=7.7 Hz, H-c), 7.54 (d, 1H, J=5.0 Hz, aromatic proton), 7.59 (d, 1H, J=3.2 Hz, aromatic proton), 7.75 (d, 1H, J=6.9 Hz, H-a), 8.28 (d, 1H, J=8.8 Hz, H-d), 10.00 (s, 1H, —NH); ¹³C NMR (151 MHz, Chloroform-d) δ 45.2 (—NCH₂Ar), 55.5 (—OCH₃), 87.2 (C-f), 112.3 (C-b), 115.0 (C-n), 115.7 (t, J=16.2 Hz, C-p)*, 118.4 (C-d), 123.1 (C-a), 124.3 (C-l), 127.3 (thiophene carbon), 127.9 (thiophene carbon), 128.1 (t, J=3.3 Hz, C-s)*, 128.6 (C-m), 128.5 (thiophene carbon), 130.0 (t, J=5.3 Hz, thiophene carbon), 131.7 (C-c), 142.6 (C-e), 142.9 (dd, J=248.0, 15.8 Hz, C-q)**, 144.0 (d, J=247.0, Hz, C-r)**, 160.0 (C-o)***, 161.6 (C-h)***, 162.2 (C-g)***. MS (ES+): 528.2 (M+1).

1-(4-Methoxybenzyl)-2-oxo-N-(2,3,5,6-tetrafluoro-4-(pyridin-3-yl)phenyl)-1,2-dihydropyrazolo[1,5-a]pyridine-3-carboxamide (61). The crude was purified by flash chromatography (eluent: from petroleum ether/EtOAc 6/4 v/v to 3/7 v/v) obtaining a solid crude. This latter was then triturated with diisopropyl ether to obtain the title compound as a white solid (m.p. 202.6-203.8° C. from trituration with diisopropyl ether). Yield: 90%. ¹H NMR (600 MHz, Chloroform-d) δ 3.78 (s, 3H, —OCH₃), 5.41 (s, 2H, —NCH₂Ar), 6.77 (t, 1H, J=6.7 Hz, H-b), 6.89 (d, 2H, J=8.5 Hz, H-n), 7.21 (d, 2H, J=8.5 Hz, H-m), 7.43-7.49 (m, 2H, aromatic protons and H-c), 7.77 (d, 1H, J=6.9 Hz, H-a), 7.84 (d, 1H, J=7.8 Hz, aromatic proton), 8.27 (d, 1H, J=8.8 Hz, H-d), 8.69 (d, 1H, J=2.9 Hz, aromatic proton), 8.76 (s, 1H, aromatic proton), 10.06 (s, 1H, —NH); ¹³C NMR (151 MHz, Chloroform-d) δ 45.2 (—NCH₂Ar), 55.5 (—OCH₃), 87.1 (C-f), 112.9 (C-b), 114.0 (t, J=16.9 Hz, C-p)*, 115.0 (C-n), 117.4 (t, J=15.8 Hz, C-s)*, 118.3 (C-d), 123.1 (C-a), 123.7 (pyridine carbon), 124.2 (C-l), 124.3 (pyridine carbon), 128.6 (C-m), 131.8 (C-c), 137.9 (pyridine carbon), 142.6 (C-e), 142.9 (d, J=252.7 Hz, C-q)**, 144.2 (d, J=249.7 Hz, C-r)**, 149.9 (pyridine carbon), 150.5 (pyridine carbon), 160.0 (C-o)***, 161.6 (C-h)***, 162.2 (C-g)***; MS (ES+): 523 (M+1).

1-(4-Methoxybenzyl)-2-oxo-N-(2,3,5,6-tetrafluoro-4-(2-(trifluoromethyl)pyridin-4-yl)phenyl)-1,2-dihydropyrazolo[1,5-a]pyridine-3-carboxamide (62). The crude was purified by flash chromatography (eluent: from petroleum ether/EtOAc 6/4 v/v to 3/7 v/v) obtaining a solid crude. This latter was then triturated with diisopropyl ether and obtain the title compound as a pale yellow solid (m.p 192.7-193.9° C. from trituration with diisopropyl ether). Yield: 70%. ¹H NMR (600 MHz, Chloroform-d) δ 3.79 (s, 3H, —OCH₃), 5.42 (s, 2H, —NCH₂Ar), 6.79 (t, 1H, J=6.7 Hz, H-b), 6.90 (d, 2H, J=8.5 Hz, H-n), 7.22 (d, 2H, J=8.5 Hz, H-m), 7.48 (t, 1H, J=7.9 Hz, H-c), 7.64 (d, 1H, J=4.9 Hz, pyridine proton), 7.78 (d, 1H, J=6.9 Hz, H-a), 7.84 (s, 1H, pyridine proton), 8.29 (d, 1H, J=8.8 Hz, H-d), 8.88 (d, 1H, J=4.9 Hz, pyridine proton), 10.15 (s, 1H, —NH); ¹³C NMR (151 MHz, Chloroform-d) δ 45.3 (—NCH₂Ar), 55.5 (—OCH₃), 87.1 (C-f), 113.0 (C-b), 113.2 (t, J=15.6 Hz, C-p)*, 115.0 (C-n), 118.4 (C-d), 118.9 (t, J=14.7 Hz, C-s)*, 121.5 (q, J=274.5 Hz, —CF₃), 121.7 (q, J=2.1 Hz, pyridine carbon), 123.1 (C-a), 124.2 (C-l), 127.6 (pyridine carbon), 128.6 (C-m), 131.9 (C-c), 137.9 (pyridine carbon), 142.6 (C-e), 142.8 (d, J=247.0 Hz, C-q)**, 144.2 (d, J=245.4 Hz, C-r)**, 149.0 (q, J=34.9 Hz, pyridine carbon), 150.6 (pyridine carbon), 160.1 (C-o)***, 161.4 (C-h)***, 162.3 (C-g)***; MS (ES+): 591.

1-(4-Methoxybenzyl)-2-oxo-N-(2,3,5,6-tetrafluoro-3′-hydroxy-[1,1′-biphenyl]-4-yl)-1,2-dihydropyrazolo[1,5-a]pyridine-3-carboxamide (63). The crude was purified by flash chromatography (eluent: petroleum ether/EtOAc 1/2 v/v) obtaining a solid crude. This latter was then triturated with diisopropyl ether, in order to remove O═PPh₃O, obtaining the title compound as a white solid (236.9-237.4° C. from diisopropyl ether). Yield: 79%. ¹H NMR (600 MHz, DMSO-d₆) δ 3.71 (s, 3H, —OCH₃), 5.49 (s, 2H, —NCH₂Ar), 6.88-6.97 (m, 5H, aromatic protons and H-n), 7.07 (t, 1H, J=7.1 Hz, H-b), 7.28 (d, 2H, J=8.3 Hz, H-m), 7.35 (t, 1H, J=8.1 Hz, aromatic proton), 7.67 (t, 1H, J=7.9 Hz, H-c), 8.03 (d, 1H, J=8.8 Hz, H-d), 8.57 (d, 1H, J=6.9 Hz, H-a), 9.78 (br s, 1H, —OH), (s, 1H, —NH); ¹³C NMR (151 MHz, DMSO-d₆) δ 43.8 (—NCH₂Ar), 55.1 (—OCH₃), 85.5 (C-f), 113.2 (C-b), 114.4 (C-n), 116.2 (C-d), 116.3 (aromatic carbon), 116.6 (t, J=14.6 Hz, C-p)*, 116.9 (2 carbon, C-s partially overlapped with aromatic carbon)*, 120.7 (aromatic carbon), 125.3 (C-a), 125.4 (C-l), 127.7 (aromatic carbon), 128.8 (C-m), 129.9 (aromatic carbon), 132.9 (C-c), 141.4 (C-e), 142.3 (d, J=248.9 Hz, C-q)**, 143.3 (d, J=251.3 Hz, C-r)**, 157.5 (aromatic carbon), 159.1 (C-o)***, 160.7 (C-h)***, 161.7 (C-g)***; MS (ES+): 538 (M+1).

1-(4-Methoxybenzyl)-2-oxo-N-(2,3,5,6-tetrafluoro-3′-(trifluoromethoxy)-[1,1′-biphenyl]-4-yl)-1,2-dihydropyrazolo[1,5-a]pyridine-3-carboxamide (64) The crude was purified by flash chromatography (eluent: petroleum ether/DCM 2/1 v/v) obtaining a solid crude. This latter was then triturated with diisopropyl ether obtaining the title compound as a white solid (162.7-163.0° C. from diisopropyl ether). Yield: 82%. ¹H NMR (600 MHz, Chloroform-d) δ 3.80 (s, 3H, —OCH₃), 5.42 (s, 2H, —NCH₂Ar), 6.77 (t, 1H, J=6.9 Hz, H-b), 6.91 (d, 2H, J=8.5 Hz, H-n), 7.22 (d, 2H, J=8.5 Hz, H-m), 7.32 (d, 1H, J=8.1 Hz, aromatic proton), 7.37 (s, 1H, aromatic proton), 7.43 (d, 1H, J=7.7 Hz, aromatic proton), 7.47 (t, 1H, J=7.9 Hz, H-c), 7.53 (t, 1H, J=8.0 Hz, aromatic proton), 7.76 (d, 1H, J=6.9 Hz, H-a), 8.31 (d, 1H, J=8.8 Hz, H-d), 10.03 (s, 1H, —NH); ¹³C NMR (151 MHz, Chloroform-d) δ 45.2 (—NCH₂Ar), 55.4 (—OCH₃), 87.1 (C-f), 112.8 (C-b), 115.0 (C-n), 115.9 (t, J=16.1 Hz, C-p)*, 117.0 (t, J=15.6 Hz, C-s)*, 118.3 (C-d), 120.6 (q, J=257.6 Hz, —OCF 3), 121.5 (aromatic carbon), 123.0 (aromatic carbon), 123.1 (C-a), 124.2 (C-l), 128.5 (C-m), 128.8 (aromatic carbon), 129.4 (aromatic carbon), 130.1 (aromatic carbon), 131.7 (C-c), 142.5 (C-e), 142.8 (d, J=247.5 Hz, C-q)**, 144.0 (d, J=249.5 Hz, C-r)**, 149.3 (aromatic carbon), 160.0 (C-o)***, 161.6 (C-h)***, 162.2 (C-g)***; MS (ES+): 606.6, 628.6.

1-(4-Methoxybenzyl)-2-oxo-N-(2,3,5,6-tetrafluoro-3′-methoxy-[1,1′-biphenyl]-4-yl)-1,2-dihydropyrazolo[1,5-a]pyridine-3-carboxamide (65) The crude was purified by flash chromatography (eluent: petroleum ether/EtOAc/dichloromethane 1.5/1/2 v/v/v) obtaining the title compound as a beige solid (172.9-173.8° C. from diisopropyl ether). Yield: 94%. ¹H-NMR (600 MHz, Chloroform-d): δ 3.79 (s, 3H, —OCH₃), 3.85 (s, 3H, —OCH₃), (s, 2H, —NCH₂Ar), 6.76 (t, 1H, J=6.9 Hz, H-b), 6.90 (d, 2H, J=8.5 Hz, H-n), 6.97-7.03 (m, 2H, aromatic protons), 7.06 (d, 1H, J=7.5 Hz, aromatic proton), 7.22 (d, 2H, J=8.5 Hz, H-m), 7.41 (t, 1H, J=7.9 Hz, aromatic proton), 7.45 (t, 1H, J=7.9 Hz, H-c), 7.75 (d, 1H, J=6.9 Hz, H-a), 8.29 (d, 1H, J=8.8 Hz, H-d), 9.99 (s, 1H, —NH); ¹³C-NMR (150 MHz, Chloroform-d): δ 45.2 (—NCH₂Ar), 55.5 (2× —OCH₃), 87.2 (C-f), 112.3 (C-b), 114.9 (aromatic carbon), 115.0 (C-n), 115.8 (aromatic carbon), 116.3 (t, J=15.0 Hz, C-p)*, 117.6 (t, J=19.6 Hz, C-s)*, 118.4 (C-d), 122.7 (aromatic carbon), 123.1 (C-a), 124.3 (C-l), 128.6 (C-m), 128.8 (aromatic carbon), 129.7 (aromatic carbon), 131.7 (C-c), 142.6 (C-e), 142.9 (d, J=248.6 Hz, C-g)**, 144.2 (d, J=244.2, Hz, C-r)**, 159.7 (aromatic carbon)***, 160.0 (C-o)***161.7 (C-h)****, 162.2 (C-g)****; MS (ES+): 552.5 (M+1).

1-(4-Methoxybenzyl)-2-oxo-N-(2,3,5,6-tetrafluoro-3′-propoxy-[1,1′-biphenyl]-4-yl)-1,2-dihydropyrazolo[1,5-a]pyridine-3-carboxamide (66) The crude was purified by flash chromatography (eluent: petroleum ether/EtOAc 2/3 v/v) to obtain the title compound as a beige solid (168.8-169.9° C. from diisopropyl ether). Yield: 86%. ¹H NMR (600 MHz, Chloroform-d) δ 1.06 (t, 3H, J=7.4 Hz, —OCH₂CH₂CH₃), 1.84 (h, 2H, J=7.3 Hz, —OCH₂CH₂CH₃), 3.80 (s, 3H, —OCH₃), 3.96 (t, 2H, J=6.6 Hz, —OCH₂CH₂CH₃), 5.42 (s, 2H, —NCH₂Ar), 6.76 (t, 1H, J=7.0 Hz, H-b), 6.91 (d, 2H, J=8.6 Hz, H-n), 6.99 (dd, 1H, J=8.4, 2.2 Hz, aromatic proton), 7.00 (s, 1H, aromatic proton), 7.04 (d, 1H, J=7.6 Hz, aromatic proton), 7.22 (d, 2H, J=8.6 Hz, H-m), 7.39 (t, 1H, J=7.9 Hz, aromatic proton), 7.46 (t, 1H, J=7.9 Hz, H-c), 7.75 (d, 1H, J=7.0 Hz, H-a), 8.31 (d, 1H, J=8.8 Hz, H-d), 9.98 (s, 1H, —NH); ¹³C NMR (151 MHz, Chloroform-d) δ 10.6 (—OCH₂CH₂ CH₃), 22.7 (—OCH₂CH₂CH₃), 45.2 (—NCH₂Ar), 55.5 (—OCH₃), 69.8 (—OCH₂CH₂CH₃), 87.2 (C-f), 112.8 (C-b), 115.0 (C-n), 115.5 (aromatic carbon), 116.2 (t, J=15.6 Hz, C-p)*, 116.4 (aromatic carbon), 117.7 (t, J=16.8 Hz, C-s)*, 118.3 (C-d), 122.5 (aromatic carbon), 123.1 (C-a), 124.3 (C-l), 128.6 (C-m), 128.7 (aromatic carbon), 129.7 (aromatic carbon), 131.7 (C-c), 142.5 (C-e), 142.8 (dd, J=248.5, Hz, C-q)**, 144.2 (d, J=248.5 Hz, C-r)**, 159.2 (aromatic carbon)***, 160.0 (C-o)***161.7 (C-h)****, 162.2 (C-g)****; MS (ES+): 580 (M+1).

General Procedure for the Synthesis of Aniline 47-50.

Pd(PPh₃)₄ (90 mg, 0.08 mmol, 0.20 eq) was added to a solution of 4-bromo-2,3,5,6-tetrafluoroaniline (200 mg, mmol, 1.00 eq) and K₂CO₃ (158 mg, 1.14 mmol, 3.00 eq) in 1,2-dimethoxyethane (35 mL). After stirring the resulting mixture under atmosphere of nitrogen for 1 h at r.t. the corresponding boronic acid (0.760 mmol, 2.0 eq) was added; the reaction mixture was then heated at reflux under atmosphere of nitrogen. After 2 h, an additional amount of boronic acid (0.38 mmol, 1.0 eq) was added, the reaction mixture was heated at reflux for another 2 h before cooling to room temperature and concentrating under reduced pressure. The crude material was taken-up with water (100 mL) and the mixture was extracted with EtOAc (3×60 mL), the combined organic layers were dried over Na₂SO₄ and concentrated under reduced pressure. The crude was purified by flash chromatography (see below the conditions).

2,3,4′,5,6-pentafluoro-[1,1′-biphenyl]-4-aniline (47). Flash chromatography (eluent: petroleum ether/EtOAc from 95:5 v/v). White solid (m.p. 134.1-134.7° C. from trituration with diisopropyl ether). Yield 74%. ¹H NMR (600 MHz, Chloroform-d): δ 4.05 (s, 2H, —NH₂), 7.11-7.17 (m, 2H, aromatic protons), 7.37-7.42 (m, 2H, aromatic protons); ¹³C NMR (151 MHz, CDCl₃): δ 107.3 (t, J=16.8 Hz), 115.7 (d, J=21.7 Hz), 124.0 (d, J=1.7 Hz), 125.6 (t, J=14.1 Hz), 132.2 (d, J=7.7 Hz), 136.9 (d, J=238.0 Hz), 144.1 (d, J=243.5 Hz), 162.7 (d, J=248.3 Hz). MS (ES−) 258 (M−1).

2,3,3′,5,6-pentafluoro-[1,1′-biphenyl]-4-aniline (48). Flash chromatography (eluent: petroleum ether/EtOAc from 95:5 v/v). White solid (m.p. 108.8-109.7° C. from trituration with diisopropyl ether). Yield 90%. ¹H NMR (600 MHz, Chloroform-d): δ 4.08 (s, 2H, —NH 2), 7.10 (t, 1H, J=8.4 Hz aromatic proton), 7.15 (d, 1H, J=9.7 Hz, aromatic protons), 7.21 (d, 1H, J=7.5 Hz, aromatic proton), 7.38-7.44 (m, 1H, aromatic proton); ¹³C NMR (151 MHz, CDCl₃): δ 107.0 (t, J=17.6 Hz), 115.3 (d, J=20.8 Hz), 117.5 (d, J=22.3 Hz), 125.9 (t, J=14.2 Hz), 126.2, 130.0 (d, J=8.3 Hz), 130.1, 136.8 (dd, J=238.4, 16.2 Hz), 144.1 (d, J=243.9 Hz), 162.7 (d, J=245.9 Hz). MS (ES−) 258 (M−1).

2,3,5,6-tetrafluoro-4′-(trifluoromethyl)-[1,1′-biphenyl]-4-aniline (49). Flash chromatography (eluent: petroleum ether/EtOAc from 95:5 v/v). White solid (m.p. 163.9-164.8° C. from trituration with diisopropyl ether). Yield 60%. ¹H NMR (600 MHz, Chloroform-d): δ 4.12 (s, 2H, —NH 2), 7.56 (d, 2H, J=8.0 Hz, aromatic protons), 7.71 (d, 2H, J=8.2 Hz, aromatic protons); ¹³C NMR (151 MHz, CDCl₃): δ 106.8 (t, J=15.1 Hz), 124.1 (q, J=271.7 Hz, —CF 3), 125.5 (q, J=6.7 Hz), 126.4 (t, J=3.8 Hz), 130.4 (q, J=32.7 Hz), 130.8, 132.0, 136.9 (d, J=238.7 Hz), 144.2 (d, J=244.2 Hz). MS (ES−) 308 (M−1).

2,3,5,6-tetrafluoro-3′-(trifluoromethyl)-[1,1′-biphenyl]-4-aniline (50). Flash chromatography (eluent: petroleum ether/EtOAc from 95:5 v/v). Pale yellow solid (m.p. 46.6-47.0° C. from trituration with diisopropyl ether). Yield 92%. ¹H NMR (600 MHz, Chloroform-d): δ 4.11 (s, 2H, —NH 2), 7.55-7.60 (m, 1H, aromatic proton), 7.61 (d, 1H, J=7.2 Hz, aromatic proton), 7.65 (d, 1H, J=7.4 Hz, aromatic proton), 7.70 (s, 1H, aromatic proton); ¹³C NMR (151 MHz, CDCl₃): δ 106.7 (t, J=16.6 Hz), 124.1 (q, J=272.7 Hz, —CF 3), 125.2 (q, J=3.8 Hz), 126.3 (t, J=14.3 Hz), 127.3, 129.0, 129.1, 131.5 (q, J=32.7 Hz), 133.8, 136.9 (dd, J=239.4, 16.5 Hz), 144.2 (d, J=244.2 Hz). MS (ES−) 308 (M−1).

### General Procedure for the Synthesis of Pyrazolo[1,5-a]Pyridine Related Amides 52-55

Oxalyl chloride (3.0 mmol) and dry DMF (1 drop) were added to a cooled (0° C.) solution of the 0-protected pyrazolo[1,5-a]pyridine acid (1.0 mmol) 45 in dry THF (20 mL), under a nitrogen atmosphere. The obtained solution was stirred at room temperature for 2 hours. The solution was then concentrated under reduced pressure and the residue dissolved in dry THF (10 mL, this step was repeated three times). The resulting acyl chloride was immediately used without any further purification and dissolve in 10 mL of dry toluene and transferred to the solution described later. Trimethylaluminium (2.0 M in hexane, 1.5 mmol), was added to a solution of the appropriate aniline (see supporting info for the synthesis, 1.5 mmol), in dry toluene (15 mL), under a nitrogen atmosphere. The resulting mixture was stirred for 2 hours at room temperature producing a brown suspension, then the solution of the previously described acyl chloride in dry toluene (30 mL) was quantitatively added. The mixture was heated overnight at 90° C. and then cooled to r.t. The reaction was quenched with 1M HCl. The layers were resolved, and the aqueous phase was exhaustively extracted using EtOAc. The combined organic layer was washed with 1M NaOH and brine, dried and the solvent was evaporated under reduced pressure. The crude product was purified by column chromatography.

1-Benzyl-2-oxo-N-(2,3,4′,5,6-pentafluoro-[1,1′-biphenyl]-4-yl)-1,2-dihydropyrazolo[1,5-a]pyridine-3-carboxamide (52). 2,3,4′,5,6-Pentafluoro-[1,1′-biphenyl]-4-aniline was used. flash chromatography (eluent: petroleum ether/EtOAc from 80:20 v/v to 50:50 v/v). Grey solid (m.p. 234.2-235.4° C. from trituration with diisopropyl ether). Yield 31%. ¹H NMR (600 MHz, Chloroform-d): δ 5.48 (s, 2H, —NCH₂ Ph), 6.77 (t, 1H, J=6.9 Hz, H-b), 7.19 (t, 2H, J=8.6 Hz, aromatic protons), 7.28 (d, 2H, J=7.4 Hz, aromatic protons), 7.32-750 (m, 6H, aromatic protons), 7.73 (d, 1H, J=6.9, H-a), 8.30 (d, 1H, J=8.8, H-d), 9.98 (s, 1H, —NH); ¹³C NMR (151 MHz, Chloroform-d): δ 45.7 (—NCH₂Ph), 87.2 (C-f), 112.9 (C-b), 115.9 (d, J=21.9 Hz, aromatic carbon), 116.4 (t, J=17.1 Hz, C-p)*, 116.7 (d, J=16.7 Hz, C-s)*, 118.4 (C-d), 123.0 (C-a), 123.5 (aromatic carbon), 127.1 (aromatic carbon), 129.0 (aromatic carbon), 129.7 (aromatic carbon), 131.8 (C-c), 132.8 (d, J=8.4 Hz, aromatic carbon), 132.5 (aromatic carbon), 142.6 (C-e), 142.8 (d, J=244.1 Hz, C-q)**, 144.2 (d, J=250.7 Hz, C-r)**, 161.7 (C-g)***, 162.2 (C-h)***, 163.1 (d, J=249.5 Hz, —CF); MS (ESI) 510 (M+1).

1-Benzyl-2-oxo-N-(2,3,3′,5,6-pentafluoro-[1,1′-biphenyl]-4-yl)-1,2-dihydropyrazolo[1,5-a]pyridine-3-carboxamide (53). 2,3,3′,5,6-Pentafluoro-[1,1′-biphenyl]-4-aniline was used. flash chromatography (eluent: petroleum ether/EtOAc from 80:20 v/v to 50:50 v/v). Pale yellow solid (m.p. 195.4-196.3° C. from trituration with diisopropyl ether). Yield 40%. ¹H NMR (600 MHz, DMSO-d₆): δ 5.57 (s, 2H, —NCH₂Ph), 7.07 (t, 1H, J=6.9 Hz, H-b), 7.29-7.45 (m, 7H, aromatic protons), 7.50 (d, 1H, J=9.5 Hz, aromatic proton), 7.62 (dd, 1H, J=14.3, 7.7 Hz, aromatic proton), 7.68 (t, 1H, J=7.9 Hz, H-c), 8.05 (d, 1H, J=8.7 Hz, H-d), 8.53 (d, 1H, J=7.0 Hz, H-a), 10.12 (s, 1H, —NH); ¹³C NMR (151 MHz, DMSO-d₆): δ 44.2 (—NCH₂Ph), 85.4 (C-f), 113.3 (C-b), 115.4 (t, J=17.3 Hz, C-p)*, 116.2 (C-d), 116.4 (d, J=20.8 Hz, aromatic carbon), 117.0 (C-s)*, 117.2 (d, J=23.1 Hz, aromatic carbon), 125.2 (C-a), 126.5 (C-l), 127.2 (aromatic carbon), 128.2 (C-c), 128.8 (d, J=9.8 Hz, aromatic carbon), 129.1 (aromatic carbon), 130.9 (d, J=8.4 Hz, aromatic carbon), 132.9 (aromatic carbon), 133.7 (aromatic carbon), 141.4 (C-e), 142.2 (d, J=242.5 Hz, C-q)**, 143.3 (d, J=245.8 Hz, C-r)**, 144.1, 160.6 (C-g)***, 161.2 (C-h)***, 162.0 (d, J=244.3 Hz, aromatic carbon); MS (ESI) 508 (M−1).

1-Benzyl-2-oxo-N-(2,3,5,6-tetrafluoro-4′-(trifluoromethyl)-[1,1′-biphenyl]-4-yl)-1,2-dihydropyrazolo[1,5-a]pyridine-3-carboxamide (54). 2,3,5,6-Tetrafluoro-4′-(trifluoromethyl)-[1,1′-biphenyl]-4-aniline was used; flash chromatography eluent: from petroleum ether/EtOAc 80:20 v/v to 50:50 v/v. Pale yellow solid (m.p. 262.2-263.5° C. from trituration with diisopropyl ether). Yield 41%. ¹H NMR (600 MHz, Chloroform-d): δ 5.49 (s, 2H, —NCH₂Ph), 6.78 (t, 1H, J=6.8 Hz, H-b), 7.29 (d, 2H, J=7.4 Hz, aromatic protons), 7.33-7.43 (m, 3H, aromatic protons), 7.48 (t, 1H, J=7.9 Hz, H-c), 7.62 (d, 2H, J=7.9 Hz, aromatic protons), 7.73 (d, 1H, J=6.9 Hz, H-a), 7.76 (d, 2H, J=8.1 Hz, aromatic protons), 8.31 (d, 1H, J=8.8 Hz, H-d), 10.04 (s, 1H, —NH); ¹³C NMR (151 MHz, Chloroform-d): δ 45.7 (—NCH₂Ph), 87.2 (C-f), 112.9 (C-b), 116.1 (t, J=16.5 Hz, C-p)*, 117.2 (t, J=15.6 Hz, C-s)*, 118.4 (C-d), 123.0 (C-a), 124.0 (q, J=272.5 Hz, —CF₃), 125.7 (q, J=3.8 Hz, aromatic carbon), 127.1 (aromatic carbon), 129.0 (C-c), 129.7 (aromatic carbon), 130.8 (aromatic carbon), 131.1 (q, J=32.8 Hz, aromatic carbon), 131.4 (aromatic carbon), 131.9 (aromatic carbon), 132.5 (aromatic carbon), 142.6 (C-e), 142.8 (dd, J=251.8, 17.4 Hz, C-q)**, 144.1 (dd, J=244.7, 15.4 Hz, C-r)**, 161.6 (C-g)***, 162.2 (C-h)***; MS (ESI) 560 (M+1).

1-Benzyl-2-oxo-N-(2,3,5,6-tetrafluoro-3′-(trifluoromethyl)-[1,1′-biphenyl]-4-yl)-1,2-dihydropyrazolo[1,5-a]pyridine-3-carboxamide (55). 2,3,5,6-Tetrafluoro-3′-(trifluoromethyl)-[1,1′-biphenyl]-4-aniline was used; flash chromatography eluent: from petroleum ether/EtOAc 80:20 v/v to 50:50 v/v. White solid (m.p. 190.9-191.8° C. from trituration with diisopropyl ether). Yield 40%. ¹H NMR (600 MHz, Chloroform-d): δ 5.49 (s, 2H, —NCH₂Ph), 6.78 (t, 1H, J=6.8 Hz, H-b), 7.29 (d, 2H, J=7.3 Hz, aromatic protons), 7.33-7.42 (m, 3H, aromatic protons), 7.48 (t, 1H, J=7.9 Hz, H-c), 7.64 (t, 1H, J=7.7 Hz, aromatic proton), 7.68 (d, 1H, J=7.5 Hz, aromatic proton), 7.72 (t, 2H, J=7.6 Hz, aromatic protons), 7.76 (s, 1H, aromatic proton), 8.31 (d, 1H, J=8.8, H-d), 10.04 (s, 1H, —NH); ¹³C NMR (151 MHz, Chloroform-d): δ 45.7 (—NCH₂Ph), 87.2 (C-f), 112.9 (C-b), 116.4 (t, J=16.2 Hz, C-p)*, 117.2 (t, J=15.2 Hz, C-s)*, 118.5 (C-d), 123.0 (C-a), 124.0 (q, J=272.1 Hz, —CF 3), 125.9 (q, J=3.9 Hz, aromatic carbon), 127.1 (aromatic carbon), 127.2 (aromatic carbon), 128.5 (aromatic carbon), 129.0 (aromatic carbon), 129.3 (aromatic carbon), 129.7 (aromatic carbon), 131.3 (q, J=32.4 Hz), 131.8 (C-c), 132.5 (aromatic carbon), 133.7 (aromatic carbon), 142.7 (C-e), 142.8 (d, J=252.0 Hz, C-q)**, 144.2 (d, J=248.2 Hz, C-r)**, 161.6 (c-g)***, 162.2 (C-h)***; MS (ESI) 560 (M+1).

General Procedure: removal of the 4-MeObenzyloxy moiety for the production of cpds 7-9, 15-17. Thioanisole (220 μL, 1.87 mmol, from 5.0 eq to 10 equivalent) was added to a solution of corresponding starting material (200 mg, 0.37 mmol, 1.0 eq) in TFA (3 mL). The mixture was heated at 70° C. for 2 h then cooled to r.t. The mixture was partially concentrated, and the crude was taken up with water obtaining a suspension that was filtered, the solid washed with additional amount of cold water. The resulting solid was triturated with diisopropyl ether to afford the title compounds, often directly in pure form (see details above).

2-Hydroxy-N-(2,3,5,6-tetrafluoro-4-morpholinophenyl)pyrazolo[1,5-a]pyridine-3-carboxamide (6). Compound 59 (200 mg, 0.38 mmol, 1.0 eq) was dissolved in a solution of thioanisole (250 μL, 2.26 mmol, 6.0 eq) in TFA (2 mL). The residue was triturated with hexane and diisopropyl ether, then purified by flash chromatography (eluent: petroleum ether/DCM/MeOH 5/4/0.4 v/v/v). The resulting crude solid was triturated with diisopropyl ether to afford the title compound as white solid (276.5-277.2° C. dec. from diisopropyl ether). Yield: 42%. ¹H NMR (600 MHz, DMSO-d₆) δ 3.18-3.25 (m, 4H, —NCH₂ CH₂O—), 3.68-3.75 (m, 4H, —NCH₂ CH₂O—), 7.0 (t, 1H, J=6.8 Hz, H-b), 7.48 (t, 1H, J=7.8 Hz, H-c), 7.94 (d, 1H, J=8.7 Hz, H-d), 8.58 (d, 1H, J=6.7 Hz, H-a), 8.76 (s, 1H, —NH), 12.83 (v br s, 1H, —OH); ¹³C NMR (151 MHz, DMSO-d₆) δ 51.0 (—NCH₂CH₂O—), 66.7 (—NCH₂CH₂O—), 88.2 (C-f), 111.5 (t, J=15.1 Hz, C-p)*, 113.1 (C-b), 116.8 (C-d), 127.5 (t, J=11.2 Hz, C-s)*, 128.2 (C-a), 129.1 (C-c), 141.7 (C-e), 142.2 (dd, J=243.5, 6.2 Hz, C-g)**, 143.4 (dd, J=244.6, 14.4 Hz, C-r)**, 160.8 (C-h)***, 162.7 (C-g)***. MS (ES−): 409 (M−1). ESI-HRMS (m/z): [M−H]⁻ calcd for C₁₈H₁₃F₄N₄O₃, 409.0929; obsd, 409.0925.

2-Hydroxy-N-(2,3,5,6-tetrafluoro-4-(thiophen-2-yl)phenyl)pyrazolo[1,5-a]pyridine-3-carboxamide (7). The solid was crystallized three times from acetonitrile (20 mL) to obtain the title compound as a grey solid (278.4-279.9° C. dec. from acetonitrile). Yield: 23%. ¹H NMR (600 MHz, DMSO-d₆) δ 7.03 (t, 1H, J=6.6 Hz, H-b), 7.31 (t, 1H, J=4.3 Hz, aromatic proton), 7.51 (t, 1H, J=7.8 Hz, H-c), 7.64 (d, 1H, J=2.8 Hz, aromatic proton), 7.92 (d, 1H, J=5.0 Hz, aromatic proton), 7.97 (d, 1H, J=8.8 Hz, H-d), 8.61 (d, 1H, J=6.7 Hz, H-a), 8.95 (s, 1H, —NH), 12.90 (br s, 1H, —OH); ¹³C NMR (151 MHz, DMSO-dd δ 88.2 (C-f), 110.9 (t, J=15.6 Hz, C-p)*, 113.8 (C-b), 17.03 (t, J=16.5 Hz, C-s)*, 116.8 (C-d), 126.2 (thiophene carbon), 127.8 (thiophene carbon), 128.4 (C-a), 129.2 (thiophene carbon), 129.5 (C-c), 130.7 (t, J=4.0 Hz, thiophene carbon), 141.8 (C-e), 143.0 (dd, J=246.5, 14.3 Hz, C-q and C-r), 160.4 (C-h)**, 162.7 (C-g)**. MS (ES−): 406 (M−1). ESI-HRMS (m/z): [M−H]⁻ calcd for C₁₈H₈F₄N₃O₂S, 406.0279; obsd, 406.0275.

2-Hydroxy-N-(2,3,5,6-tetrafluoro-4-(pyridin-3-yl)phenyl)pyrazolo[1,5-a]pyridine-3-carboxamide (8). Pale yellow solid (m.p. 283.4-286.7° C. dec. from trituration with diisopropyl ether). Yield: 76%. ¹H NMR (600 MHz, DMSO-d₆) δ 6.98 (t, 1H, J=6.7 Hz, H-b), 7.47 (t, 1H, J=7.9 Hz, H-c), 7.61 (dd, 1H, J=7.7, 5.0 Hz, aromatic proton), 7.95 (d, 1H, J=8.6 Hz, H-d), 8.05 (d, 1H, J=7.4 Hz, aromatic proton), 8.57 (d, 1H, J=6.5 Hz, H-a), 8.71 (d, 1H, J=4.1 Hz, aromatic proton), 8.78 (s, 1H, aromatic proton), 9.30 (br s, 1H, —NH); ¹³C NMR (151 MHz, DMSO-d₆) δ 88.3 (C-f), 112.9 (C-b), 113.8 (t, J=17.4 Hz, C-p)*, 116.6 (C-d), 117.9 (t, J=14.7 Hz, C-s)*, 123.3 (pyridine carbon), 124.0 (pyridine carbon), 128.0 (C-a), 128.9 (C-c), 137.9 (pyridine carbon), 141.7 (C-e), 142.6 (dd, J=245.2, 15.1 Hz, C-q)**, 143.4 (dd, J=245.7, 17.2 Hz, C-q)**, 150.2 (pyridine carbon), 150.3 (pyridine carbon), 160.6 (C-h)***, 163.9 (C-g)***. MS (ES+): 403 (M+H). ESI-HRMS (m/z): [M+H]⁺ calcd for C₁₉H₁₁F₄N₄O₂, 403.0813; obsd, 403.0810.

2-Hydroxy-N-(2,3,5,6-tetrafluoro-4-(2-(trifluoromethyl)pyridin-4-yl)phenyl)pyrazolo[1,5-a]pyridine-3-carboxamide (9). Pale yellow solid (m.p. 258.4-259.2° C. dec. from trituration with diisopropyl ether). Yield: 55%. ¹H NMR (600 MHz, DMSO-d₆) δ 7.01 (t, 1H, J=6.6 Hz, H-b), 7.49 (t, 1H, J=7.7 Hz, H-c), 7.96 (d, 1H, J=8.6 Hz, H-d), 8.00 (d, 1H, J=3.5 Hz, aromatic proton), 8.22 (s, 1H, aromatic proton), 8.59 (d, 1H, J=6.6 Hz, H-a), 8.99 (d, J=4.6 Hz, 1H, aromatic proton), 9.22 (br s, 1H, —NH); n C NMR (151 MHz, DMSO-d₆) δ 88.2 (C-f), 113.1 (C-p* overlapped with C-b), 116.7 (C-d), 118.9 (t, J=13.5 Hz, C-s)*, 121.5 (q, J=274.6 Hz, —CF 3), 121.8 (pyridine carbon), 128.2 (C-a), 128.5 (C-c), 129.1 (q, J=12.5 Hz, pyridine carbon), 137.3 (pyridine carbon), 141.7 (C-e), 142.6 (d, J=246.8 Hz, C-q)**, 143.3 (d, J=247.0 Hz, C-r)**, 147.1 (q, J=34.1 Hz, pyridine carbon), 151.0 (pyridine carbon), 160.3 (C-h)***, 163.4 (C-g)***. MS (ES+): 470 (M+H). ESI-HRMS (m/z): [M+H]⁺ calcd for C₂₀H₁₀F₇N₄O₂, 471.0686; obsd, 471.0684.

2-Hydroxy-N-(2,3,5,6-tetrafluoro-3′-hydroxy-[1,1′-biphenyl]-4-yl)pyrazolo[1,5-a]pyridine-3-carboxamide (14). white solid (275.9-276.4° C. dec. from diisopropyl ether). Yield: 58%. ¹H NMR (600 MHz, DMSO-d₆) δ 6.84-6.99 (m, 3H, aromatic protons), 7.03 (t, 1H, J=6.8 Hz, H-b), 7.35 (t, 1H, J=8.2 Hz, aromatic proton), 7.51 (t, 1H, J=7.8 Hz, H-c), 7.98 (d, 1H, J=7.4 Hz, H-d), 8.61 (d, 1H, J=4.5 Hz, H-a), 8.90 (s, 1H, —Ar—OH), 9.78 (s, 1H, —NH), 12.86 (s, 1H, —OH); ¹³C NMR (151 MHz, DMSO-d₆) δ 88.2 (C-f), 113.3 (C-b), 114.7 (t, J=13.5 Hz, C-p)*, 116.3 (C-d), 116.8 (aromatic carbon), 117.4 (t, J=16.3 Hz, C-s)*, 120.7 (aromatic carbon), 127.6 (aromatic carbon), 128.4 (C-a), 128.3 (aromatic carbon), 129.1 (C-c), 129.9 (aromatic carbon), 141.7 (C-e), 142.7 (d, J=248.4 Hz, C-q)**, 143.2 (d, J=244.8 Hz, C-r)**, 157.5 (aromatic carbon), 160.4 (C-h)***, 162.6 (C-g)***; MS (ES−): 416 (M−1). ESI-HRMS (m/z): [M+H]⁺ calcd for C₂₀H₁₂F₄N₃O₃, 418.0809; obsd, 418.0807.

2-Hydroxy-N-(2,3,5,6-tetrafluoro-3′-(trifluoromethoxy)-[1,1′-biphenyl]-4-yl)pyrazolo[1,5-a]pyridine-3-carboxamide (15). The crude was dissolved in methanol, filtered from insoluble solid, partly evaporated and precipitated with addition of diisopropyl ether. Beige solid (228.1-229.5° C. dec. from diisopropyl ether). Yield: 26%. ¹H NMR (600 MHz, DMSO-d₆) δ 7.03 (t, 1H, J=6.7 Hz, H-b), 7.51 (t, 1H, J=7.8 Hz, H-c), 7.55 (d, 1H, J=8.0 Hz, aromatic proton), 7.60-7.67 (m, 2H, aromatic protons), 7.71 (t, 1H, J=7.9 Hz, aromatic proton), 7.98 (d, 1H, J=8.7 Hz, H-d), 8.61 (d, 1H, J=6.7 Hz, H-a), 8.97 (s, 1H, —NH), 12.94 (br s, 1H, —OH); ¹³C NMR (151 MHz, DMSO-d₆) δ 88.2 (C-f), 113.2 (C-b), 115.5 (t, J=17.2 Hz, C-p)*, 116.8 (C-d), 117.6 (t, J=15.1 Hz, C-s)*, 120.1 (q, J=257.0 Hz, —OCF 3), 122.1 (aromatic carbon), 123.0 (aromatic carbon), 128.4 (C-a), 128.8 (aromatic carbon), 129.2 (C-c), 129.5 (aromatic carbon), 130.9 (aromatic carbon), 141.7 (C-e), 142.7 (dd, J=246.2, 13.6 Hz, C-q)**, 143.3 (d, J=245.6 Hz, C-r)**, 148.4 (aromatic carbon), 160.3 (C-h)***, 162.7 (C-g)***; MS (ES−): 484 (M−1). ESI-HRMS (m/z): [M+H]⁺ calcd for C₂₁H₁₁F₇N₃O₃, 486.0683; obsd, 486.0681.

2-Hydroxy-N-(2,3,5,6-tetrafluoro-3′-methoxy-[1,1′-biphenyl]-4-yl)pyrazolo[1,5-a]pyridine-3-carboxamide (16). Residue was treated with diethyl ether and purified by flash chromatography (eluent: DCM/MeOH 97/3 v/v then petroleum ether/dichloromethane/methanol 5/4/0.6 v/v/v) to obtain the title compound as a white solid (223.6-224.1° C. dec. from diisopropyl ether). Yield: 47%. ¹H NMR (600 MHz, DMSO-d₆) δ 3.81 (s, 3H, —OCH₃), 7.02 (t, 1H, J=6.6 Hz, H-b), 7.08-7.16 (m, 3H, aromatic protons), 7.45-7.54 (m, 2H, H-c and aromatic proton), 7.98 (d, 1H, J=8.6 Hz, H-d), 8.61 (d, 1H, J=6.6 Hz, H-a), 8.99 (br s, 1H, —NH); ¹³C-NMR (150 MHz, DMSO-d₆): δ 55.4 (—OCH₃), 88.2 (C-f), 113.2 (C-b), 115.0 (C-d), 115.8 (aromatic carbon), 116.8 (aromatic carbon), 117.0 (t, J=16.1 Hz, C-s and C-p)*, 122.3 (aromatic carbon), 127.8 (C-a), 128.3 (aromatic carbon), 129.1 (C-c), 130.0 (aromatic carbon), 141.8 (C-e), 142.9 (d, J=246.3 Hz, C-q)**, 143.4 (d, J=252.5 Hz, C-r)**, 159.4 (aromatic carbon), 160.5 (C-h)****, 163.0 162.2 (C-g)****; MS (ES−): 430 (M−1), MS (ES+) 432 (M+1). ESI-HRMS (m/z): [M+H]₊ calcd for C₂₁H₁₄F₄N₃O₃, 432.0966; obsd, 432.0969.

2-Hydroxy-N-(2,3,5,6-tetrafluoro-3′-propoxy-[1,1′-biphenyl]-4-yl) pyrazolo[1,5-a]pyridine-3-carboxamide (17). Beige solid (209.4-210.0° C. from diisopropyl ether). Yield: 69%. ¹H NMR (600 MHz, DMSO-d₆) δ 0.98 (t, 3H, J=7.2 Hz, —OCH₂CH₂CH₃), 1.68-1.81 (m, 2H, OCH₂CH₂CH₃), 3.98 (t, 2H, J=6.1 Hz, —OCH₂CH₂CH₃), 7.02 (t, 1H, J=6.5 Hz, H-b), 7.05-7.14 (m, 3H, aromatic protons), 7.46 (t, 1H, J=7.8 Hz, aromatic proton or H-c), 7.51 (t, 1H, J=7.6 Hz, aromatic proton or H-c), 7.99 (d, 1H, J=8.7 Hz, H-d), 8.61 (d, 1H, J=6.5 Hz, H-a), 8.91 (s, 1H, —NH), 12.86 (s, 1H, —OH); ¹³C NMR (151 MHz, DMSO-d₆) δ 10.4 (—OCH₂CH₂CH₃), 22.0 (—OCH₂CH₂CH₃), 69.2 (—OCH₂CH₂ CH₃), 88.2 (C-f), 113.3 (C-b), 115.5 (C-d), 116.2 (aromatic carbon), 116.8 (aromatic carbon), 116.9-117. 7 (m, C-s and C-p), 122.2 (aromatic carbon), 127.8 (aromatic carbon), 128.4 (C-a), 129.2 (C-c), 129.9 (aromatic carbon), 141.7 (C-e), 142.8 (dd, J=245.5, 21.8 Hz, C-q)**, 143.3 (d, J=245.6 Hz, C-r)**, 158.8 (aromatic carbon)***, 160.4 (C-h)****, 162.6 (C-g)****; MS (ES−): 458 (M−1), MS (ES+): 460 (M+1). ESI-HRMS (m/z): [M+H]⁺ calcd for C₂₃H₁₈F₄N₃O₃, 460.1279; obsd, 460.1280.

2-Hydroxy-N-(2,3,5,6-tetrafluoro-[1,1′-biphenyl]-4-yl)-4,5,6,7-tetrahydropyrazolo[1,5-a]pyridine-3-carboxamide (5). Palladium on carbon (Pd/C, 20% w/w) was added to a solution of compound 51, (1.0 mmol) in dry THF (10 mL). The resulting mixture was stirred under a hydrogen atmosphere of 40 bar, at temperature of 65° C. for 3 hours using Microwave SynthWAVE. The suspension was filtered through Celite and the cake was washed with methanol. The filtrate was concentrated under reduced pressure. The obtained solid was further purified by flash chromatography (eluent: dichloromethane/EtOAc/HCOOH v/v/v). White solid (m.p. 270.9-272.9° C. dec, from diisopropyl ether). Yield 40% ¹H NMR (600 MHz, DMSO-d 5): δ 1.70-1.80 (m, 2H, H-b), 1.89-1.98 (m, 2H, H-c), 2.92 (t, 2H, J=6.1 Hz, H-d), 3.86 (t, 2H, J=5.8 Hz, H-a), 7.49-7.55 (m, 5H, aromatic protons), 9.11 (s, 1H, —NH), 11.94 (v br s, 1H, —OH). Exchangeable proton signals overlapped with the water signal; ¹³C NMR (151 MHz, DMSO-d₆): δ 18.5 (C-b), 22.1 (C-d), 22.9 (C-c), 46.5 (C-a), 95.5 (C-f), 116.8 (t, J=14.3 Hz, C-s)*, 117.0 (t, J=17.4 Hz, C-p)*, 126.7 (aromatic carbon), 128.9 (aromatic carbon), 129.4 (aromatic carbon), 130.1 (aromatic carbon), 141.8 (C-e) 143.2 (d, J=248.4, 21.8 Hz, C-q)**, 144.3 (d, J=244.0 Hz, C-r)**, 159.7 (C-g)***, 160.7 (C-h)***; MS (ES−) 404 (M−1). IR (KBr) v (cm⁻¹): 3338, 2924, 2519, 1685, 1577, 1522, 1437, 1374, 1316, 1283, 1241, 1144, 992. ESI-HRMS (m/z): [M+H]⁺ calcd for C₂₀H₁₆F₄N₃O₂, 406.1173; obsd, 406,1170.

General Hydrogenation Procedure for the Obtainment of Target Compounds 10-13.

Palladium on carbon (Pd/C, 6% w/w), was added to a solution of the appropriate amide (compounds 52-55, 1.0 mmol), in dry THF (15 mL), and HCl (1.0 mmol). The resulting mixture was vigorously stirred under a hydrogen atmosphere for 6 hours. The suspension was filtered through Celite then the cake was washed with methanol. The filtrate was concentrated under reduced pressure. When necessary, the obtained solid was further purified by flash chromatography (see details below).

2-Hydroxy-N-(2,3,4′,5,6-pentafluoro-[1,1′-biphenyl]-4-yl)pyrazolo[1,5-a]pyridine-3-carboxamide (10). Obtained from 52, flash chromatography (eluent: dichloromethane/EtOAc/HCOOH 80:20:1 v/v/v). White solid (m.p. 293.4-294.5° C. dec. from trituration with diisopropyl ether). Yield 75%. ¹H NMR (600 MHz, DMSO-d₆): δ 7.03 (t, 1H, J=6.7 Hz, H-b), 7.42 (t, 2H, J=8.7 Hz, aromatic protons), 7.51 (t, 1H, J=7.8 Hz, H-c), 7.57-7.71 (m, 2H, aromatic protons), 7.98 (d, 1H, J=8.7, H-d), 8.62 (d, 1H, J=6.7, H-a), 8.93 (s, 1H, —NH), 12.88 (br s, 1H, —OH); ¹³C NMR (151 MHz, DMSO-d₆): δ 88.2 (C-f), 113.3 (C-b), 116.0 (d, J=22.0 Hz, aromatic carbon), 116.3 (t, J=17.4 Hz, C-p)*, 116.8 (C-d), 117.1 (t, J=14.1 Hz, C-s)*, 123.0 (aromatic carbon), 128.4 (C-a), 129.2 (C-c), 132.5 (d, J=8.4 Hz, aromatic carbon), 141.7 (C-e), 142.8 (d, J=246.4 Hz, C-q)**, 143.3 (d, J=248.6 Hz, C-r)**, 160.4 (C-g)***, 162.6 (d, J=247.1 Hz, —CF), 162.7 (C-h)***; MS (ESI) 420 (M+1). ESI-HRMS (m/z): [M+H]⁺ calcd for C₂₀H₁₁F₅N₃O₂, 420.0766; obsd, 420.0766.

2-Hydroxy-N-(2,3,3′,5,6-pentafluoro-[1,1′-biphenyl]-4-yl)pyrazolo[1,5-a]pyridine-3-carboxamide (11). Obtained from 53, flash chromatography eluent: dichloromethane/EtOAc/HCOOH 80:20:1 v/v/v. White solid (m.p. 255.4-256.2° C. dec. from trituration with diisopropyl ether). Yield 75%. ¹H NMR (600 MHz, DMSO-d₆): δ 7.03 (t, 1H, J=6.8 Hz, H-b), 7.33-7.56 (m, 4H, aromatic protons), 7.62 (dd, 1H, J=14.3, 7.7 Hz, aromatic proton), 7.99 (d, 1H, J=8.7, H-d), 8.62 (d, 1H, J=6.8, H-a), 8.94 (s, 1H, —NH), 12.83 (br s, 1H, —OH); ¹³C NMR (151 MHz, CDCl₃): δ 88.2 (C-f), 113.3 (C-b), 115.9 (t, J=17.4 Hz, C-p)*, 116.4 (d, J=20.9 Hz, aromatic carbon), 116.9 (C-d), 117.2 (d, J=23.0 Hz, aromatic carbon), 117.4 (t, J=14.8 Hz, C-s)*, 126.5 (aromatic carbon), 128.4 (C-a), 128.7 (d, J=9.6 Hz, aromatic carbon), 129.2 (C-c), 130.9 (d, J=8.3 Hz, aromatic carbon), 141.7 (C-e), 142.8 (d, J=243.6 Hz, C-q)**, 143.2 (d, J=244.6 Hz, C-r)**, 160.4 (C-g)***, 162.0 (d, J=244.4 Hz, —CF), 162.7 (C-h)***; MS (ESI) 418 (M−1). ESI-HRMS (m/z): [M+H]⁺ calcd for C₂₀H₁₁F₅N₃O₂, 420.0766; obsd, 420.0763.

2-Hydroxy-N-(2,3,5,6-tetrafluoro-4′-(trifluoromethyl)-[1,1′-biphenyl]-4-yl)pyrazolo[1,5-a]pyridine-3-carboxamide (12). Obtained from 54, flash chromatography eluent: dichloromethane/EtOAc/HCOOH 80:20:1 v/v/v. White solid (m.p. 286.1-286.8° C. dec. from trituration with diisopropyl ether). Yield 95%. ¹H NMR (600 MHz, DMSO-d₆): δ 7.03 (t, 1H, J=6.7 Hz, H-b), 7.51 (t, 1H, J=7.8 Hz, H-c), 7.83 (d, 2H, J=7.8 Hz, aromatic protons), 7.94 (d, 2H, J=8.0 Hz, aromatic protons), 7.98 (d, 1H, J=8.7 Hz, H-d), 8.61 (d, 1H, J=6.7 Hz, H-a), 9.00 (s, 1H, —NH), 12.92 (br s, 1H, —OH); ¹³C NMR (151 MHz, DMSO-d₆): δ 88.2 (C-f), 113.3 (C-b), 115.7 (t, J=16.9 Hz, C-p)*, 116.8 (C-d), 117.8 (t, J=13.1 Hz, C-s)*, 124.0 (q, J=272.4 Hz, —CF 3), 125.8 (q, J=3.3 Hz, aromatic carbon), 128.4 (C-a), 129.2 (C-c), 129.7 (q, J=32.3 Hz, aromatic carbon), 131.0 (aromatic carbon), 131.2 (aromatic carbon), 141.7 (C-e), 142.7 (dd, J=246.0, 14.9 Hz, C-q)**, 143.2 (dd, J=244.9, 19.5 Hz, C-r)**, 160.3 (C-g)***, 162.7 (C-h)***; MS (ESI) 468 (M−1). ESI-HRMS (m/z): [M+H]⁺ calcd for C₂₁H₁₁F₇N₃O₂, 470.0734; obsd, 470.0731.

2-Hydroxy-N-(2,3,5,6-tetrafluoro-3′-(trifluoromethyl)-[1,1′-biphenyl]-4-yl)pyrazolo[1,5-a]pyridine-3-carboxamide (13). Obtained from 55, flash chromatography eluent: dichloromethane/EtOAc/HCOOH 80:20:1 v/v/v. Pale pink solid (m.p. 220.3-220.7° C. dec. from trituration with diisopropyl ether). Yield 98%. ¹H NMR (600 MHz, DMSO-d₆): δ 7.03 (t, 1H, J=6.7 Hz, H-b), 7.51 (t, 1H, J=7.8 Hz, H-c), 7.82 (t, 1H, J=7.7 Hz, aromatic proton), 7.91 (d, 2H, J=7.6 Hz, aromatic protons), 7.95-8.02 (m, 2H, aromatic proton and H-d), 8.62 (d, 1H, J=6.7, H-a), 8.98 (s, 1H, —NH), 12.87 (br s, 1H, —OH); ¹³C NMR (151 MHz, DMSO-d₆): δ 88.2 (C-f), 113.3 (C-b), 115.6 (t, J=17.0 Hz, C-p)*, 116.8 (C-d), 117.6 (t, J=15.6 Hz, C-s)*, 123.9 (q, J=271.9 Hz, —CF 3), 126.2 (aromatic carbon), 126.9 (aromatic carbon), 127.9 (aromatic carbon), 128.4 (C-a), 129.2 (C-c), 129.7 (q, J=32.4 Hz, aromatic carbon), 130.1 (aromatic carbon), 134.4 (aromatic carbon), 141.7 (C-e), 143.8 (d, J=246.0 Hz, C-q)**, 143.9 (d, J=246.9 Hz, C-r)**, 160.3 (C-g)***, 162.7 (C-h)***; MS (ESI) 468 (M−1). ESI-HRMS (m/z): [M+H]⁺ calcd for C₂₁H₁₁F₇N₃O₂, 470.0734; obsd, 470.0735.

Ethyl 2-((tert-butoxycarbonyl)oxy)pyrazolo[1,5-a]pyridine-3-carboxylate (67). Cs₂CO₃ (2.86 g, 8.74 mmol) and tert-butoxycarbonyl anhydride (0.699 g, 3.2 mmol) were added to a solution of 44 (0.600 g, 2.91 mmol) in dry THF (25 mL). The reaction mixture was stirred under reflux overnight and allowed to reach room temperature. The solvent was concentrated under reduced pressure and the residue was dissolved in water (50 mL) and extracted with diethyl ether (3×50 mL). The combined organic layer was washed with brine, dried over anhydrous Na₂SO₄ and concentrated under reduced pressure. The residue was purified by flash chromatography (eluent: petroleum ether/ethyl acetate 80:20 v/v) to afford the title compound as a white solid (m.p. 95.4-96.4° C. from trituration with diisopropyl ether). Yield 93%. ¹H NMR (600 MHz, Chloroform-d₃): δ 1.39 (t, 3H, J=7.1 Hz, —OCH₂CH₃), 1.58 (s, 9H, —OC(CH₃)₃), 4.36 (q, 2H, J=7.0 Hz, —OCH₂CH₃), 6.96 (t, 1H, J=6.8 Hz, H-b), 7.42 (t, 1H, J=7.9 Hz, H-c), 8.08 (d, 1H, J=8.8 Hz, H-d), 8.38 (d, 1H, J=6.6 Hz, H-a). ¹³C NMR (151 MHz, Chloroform-d₃): δ 14.6 (—OCH₂CH₃), 27.8 (—C(CH₃)₃), 60.2 (—OCH₂CH₃), 84.8 (C-f), 93.4 (—C(CH₃)₃), 114.1 (C-b), 119.2 (C-d), 128.0 (C-a), 129.3 (C-c), 142.3 (C-e), 150.2 (C-h), 158.2 (C-g)*, 162.1 (C-i)*. MS (ESI) 307 (M+1).

Ethyl 2-((tert-butoxycarbonyl)oxy)-7-chloropyrazolo[1,5-a]pyridine-3-carboxylate (68). LiHMDS (1.0 M THF solution: 0.980 mL, 0.98 mmol, 1.5 equiv.) was added dropwise to a solution of 67 (0.400 g, 0.654 mmol) in dry THF (10 mL) cooled to a −78° C. The mixture was stirred at −78° C. for 1 h, then a solution of hexachloroethane (0.170 g, 0.72 mmol, 1.1 equiv.) in dry THF was added at −78° C. and the reaction mixture was stirred for 15 minutes at room temperature. Subsequently, the reaction was quenched with aqueous sutured solution of NH₄Cl (100 mL) The water phase was extracted with dichloromethane (4×100 mL). The combined organic phases were dried over Na₂SO₄, filtered and evaporated to dryness under vacuum. The crude product was purified by flash chromatography (eluent: petroleum ether/ethyl acetate 80:20 v/v) to afford the title compound as a white solid (m.p. 104.6-106.0° C. from trituration with diisopropyl ether). Yield 81%. ¹H NMR (600 MHz, Chloroform-d₃): δ 1.40 (t, 3H, J=7.1 Hz, —OCH₂CH₃), 1.58 (s, 9H, —OC(CH₃)₃), 4.37 (q, 2H, J=7.2 Hz, —OCH₂CH₃), 7.11 (dd, 1H, J=7.5, 1.1 Hz, H-b), 7.39 (dd, 1H, J=8.9, 7.5 Hz, H-c), 8.09 (dd, 1H, J=8.9, 1.2 Hz, H-d). ¹³C NMR (151 MHz, Chloroform-d₃): δ 14.6 (—OCH₂CH₃), 27.8 (—C(CH₃)₃), 60.5 (—OCH₂CH₃), 85.0 (C-f)₃, 95.2 (—C(CH₃)₃), 114.4 (C-b), 117.5 (C-d), 128.2 (C-c), 131.0 (C-a), 143.9 (C-e), 150.0 (C-h), 158.0 (C-g)*, 161.8 (C-i)*. MS (ESI) 241 (M+1, -Boc).

Ethyl 7-chloro-2-hydroxypyrazolo[1,5-a]pyridine-3-carboxylate (69). Trifluoroacetic acid (10 mL) was added to a solution of 68 in dry dichloromethane (25 mL) and the reaction mixture was stirred at room temperature for 4 h. The mixture was quenched with water and the layers were separated. The aqueous solution was further extracted with dichloromethane (3×25 mL). The combined organic phases were dried over Na₂SO₄, filtered and evaporated to dryness under reduced pressure. The crude product was purified by flash chromatography (eluent: dichloromethane/methanol 98:2 v/v) to afford the title compound as a white solid (m.p. 134.0-135.8° C. from trituration with diisopropyl ether). Yield 94%. ¹H NMR (600 MHz, Chloroform-d₃): δ 1.44 (t, 3H, J=7.2 Hz, —OCH₂CH₃), 4.44 (q, 2H, J=7.2 Hz, —OCH₂CH₃), 7.02 (dd, 1H, J=7.5, 1.2 Hz, H-b), 7.35 (dd, 1H, J=8.7, 7.5 Hz, H-c), 7.72 (dd, 1H, J=8.7, 1.2 Hz, H-d), 9.09 (s, 1H, —OH). ¹³C NMR (151 MHz, Chloroform-d₃): δ 14.6 (—OCH₂CH₃), 60.9 (—OCH₂CH₃), 87.9 (C-f), 113.6 (C-b), 115.5 (C-d), 128.4 (C-c), 131.3 (C-a), 142.0 (C-e), 166.0 (C-h)*, 166.9 (C-g)*. MS (ESI) 241 (M+1).

Ethyl 2-(benzyloxy)-7-chloropyrazolo[1,5-a]pyridine-3-carboxylate (70). Benzyl bromide (645 mg, 3.20 mmol, 1.10 eq) was added dropwise to a mixture of 69 (600 mg, 2.91 mmol) and Cs₂CO₃ (2.295 g, 7.04 mmol, 2.4 eq) in dry DMF (15 mL). The reaction mixture was stirred overnight at room temperature, then water (100 mL) was added. The mixture was extracted with EtOAc (4×70 mL), the combined organic layer was dried under Na₂SO₄ and evaporated under reduced pressure to get a yellow oil. The mixture was separated using flash chromatography (eluent: petroleum ether/EtOAc 6/4 v/v) to afford the title compound as a pale-yellow solid (m.p. 98.2-99.3° C. from trituration with diisopropyl ether). Yield 85% ¹H NMR (600 MHz, Chloroform-d₃): δ 1.41 (t, 3H, J=7.1 Hz, —OCH₂CH₃), 4.38 (q, 2H, J=7.1 Hz, —OCH₂CH₃), 5.58 (s, 2H, —OCH₂Ph), 6.96 (dd, 1H, J=7.4, 1.2 Hz, H-b), 7.27-7.34 (m, 2H, H-c and aromatic proton), 7.39 (t, 2H, J=7.5 Hz, aromatic protons), 7.59 (d, 2H, J=7.4 Hz, aromatic protons), 7.99 (dd, 1H, J=8.8, 1.2 Hz, H-d). ¹³C NMR (151 MHz, Chloroform-d₃): δ 14.6 (—OCH₂CH₃), 60.0 (—OCH₂CH₃), 71.1 (—OCH₂Ph), 90.1 (C-f), 112.8 (C-b), 116.5 (C-d), 127.8 (aromatic carbon), 127.9 (aromatic carbon), 128.0 (C-c), 128.5 (aromatic carbon), 130.6 (C-a), 136.7 (aromatic carbon), 144.6 (C-e), 163.1 (C-h)*, 164.8 (C-g)*. MS (ESI) 331 (M+1).

2-(Benzyloxy)-7-chloropyrazolo[1,5-a]pyridine-3-carboxylic acid (71). 6 M NaOH (5.0 eq) was added to a solution of compound 70 (785 mg, 2.40 mmol) in EtOH abs (20 mL). The mixture was stirred for 4 h at 75° C., then neutralized with 6 M HCl and concentrated under reduced pressure. The mixture was cooled 0° C. then acidified with 2 M HCl until pH 2 was reached, obtaining a suspension. This latter was filtered to get the title compound as a white solid (m.p. 178.4-179.8° C. dec. with gas developed, from trituration with diisopropyl ether). Yield 84%. ¹H NMR (600 MHz, DMSO-d₆): δ 5.48 (s, 2H, —OCH₂Ph), 7.31 (dd, 1H, J=7.5, 1.1 Hz, H-b), 7.35 (t, 1H, J=7.4 Hz, aromatic proton), 7.41 (t, 2H, J=7.4 Hz, aromatic protons), 7.52 (dd, 1H, J=8.7, 7.6 Hz, H-c), 7.55 (d, 2H, J=7.5 Hz, aromatic protons), 7.95 (dd, 1H, J=8.9, 1.1 Hz, H-d), 12.34 (br s, 1H, —COOH). ¹³C NMR (151 MHz, DMSO-d₆): δ 70.5 (—OCH₂Ph), 89.4 (C-f), 113.3 (C-b), 116.1 (C-d), 127.9 (C-a), 128.1 (aromatic carbon), 128.4 (aromatic carbon), 128.9 (aromatic carbon), 129.4 (C-c), 136.4 (aromatic carbon), 144.1 (C-e), 163.3 (C-h)*, 164.0 (C-g)*. MS (ESI) 301 (M−1).

1-Benzyl-7-chloro-2-oxo-N-(2,3,5,6-tetrafluoro-[1,1′-biphenyl]-4-yl)-1,2-dihydropyrazolo[1,5-a]pyridine-3-carboxamide (72). Oxalyl chloride (3.0 mmol) and dry DMF (1 drop) were added to a cooled (0° C.) solution of the 71 (1.0 mmol) 1-3, in dry THF (20 mL), under a nitrogen atmosphere. The obtained solution was stirred at room temperature for 2 hours. The solution was then concentrated under reduced pressure and the residue dissolved in dry THF (10 mL, this step was repeated three times). The resulting acyl chloride was immediately used without any further purification and dissolve in 10 mL of dry toluene and transferred to the solution described later. Trimethylaluminium (2.0 M in hexane, 1.5 mmol), was added to a solution of 4-phenyl-2,3,5,6-tetrafluoroaniline (1.5 mmol) in dry toluene (15 mL), under a nitrogen atmosphere. The resulting mixture was stirred for 2 hours at room temperature producing a brown suspension, then the solution of the previously described acyl chloride in dry toluene (30 mL) was quantitatively added. The mixture was heated overnight at 90° C. and then cooled to r.t. The reaction was quenched with 1M HCl, the layers resolved, and the aqueous phase was exhaustively extracted using EtOAc. The combined organic layer was washed with 1M NaOH and brine, dried and the solvent was evaporated under reduced pressure. The crude product was purified by flash chromatography (eluent: from petroleum ether/EtOAc 8:2 v/v to petroleum ether/EtOAc 4:6 v/v) to afford the title compound as a white solid (m.p. 201.1-202.4° C. from trituration with diisopropyl ether). Yield 38%. ¹H NMR (600 MHz, Chloroform-d₃): δ 5.74 (s, 2H, —NCH₂ Ph), 6.73 (dd, 1H, J=7.5, 1.2 Hz, H-b), 7.06 (dd, 2H, J=7.4, 1.7 Hz, aromatic protons), 7.22-7.30 (m, 3H, aromatic protons), 7.35 (dd, 1H, J=8.8, 7.6 Hz, H-c), 7.42-7.53 (m, 5H, aromatic protons), 8.28 (dd, 1H, J=8.8, 1.2 Hz, H-d), 10.01 (s, 1H, —NH); ¹³C NMR (151 MHz, Chloroform-d₃): δ 52.6 (—NCH₂Ph), 89.2 (C-f), 114.6 (C-b), 115.9 (t, J=16.3 Hz, C-p)*, 116.7 (C-d), 118.0 (t, J=17.2 Hz, C-s)*, 127.2 (aromatic carbon), 127.6 (C-a), 128.6 (aromatic carbon), 128.7 (aromatic carbon), 129.1 (aromatic carbon), 129.2 (aromatic carbon), 130.4 (C-c), 130.6 (aromatic carbon), 133.7 (aromatic carbon), 133.8 (aromatic carbon), 142.8 (dd, J=248.8, 15.1 Hz, C-q)**, 144.2 (d, J=248.3 Hz, C-r)**, 150.3 (C-e), 161.2 (C-g)***, 167.5 (C-h)***. MS (ESI) 526 (M−1).

7-Chloro-2-hydroxy-N-(2,3,5,6-tetrafluoro-[1,1′-biphenyl]-4-yl)pyrazolo[1,5-a]pyridine-3-carboxamide (4). Thioanisole (240 μL, 1.90 mmol, 10.0 eq) was added to a solution of 72 (100 mg, 0.19 mmol, 1.0 eq) in TFA (2 mL). The mixture was heated at 70° C. for 3 h then cooled to r.t. The mixture was partially concentrated, and the crude was taken up with water obtaining a suspension that was filtered, the solid washed with additional amount of cold water. The resulting solid was triturated with diisopropyl ether to afford the title compounds, in pure form as a white solid (m.p. 259.3-260.4° C. dec. from trituration with diisopropyl ether). Yield 64%. ¹H NMR (600 MHz, DMSO-d₆): δ 7.32 (dd, 1H, J=7.5, 1.1 Hz, H-b), 7.53 (dd, 1H, J=8.7, 7.5 Hz, H-c), 7.53-7.60 (m, aromatic protons), 8.02 (dd, 1H, J=8.8, 1.1 Hz, H-d), 8.99 (s, 1H, —NH), 13.33 (br s, 1H, —OH). ¹³C NMR (151 MHz, DMSO-d₆): δ 89.9 (C-f), 113.5 (C-b), 115.6 (C-d), 116.7 (t, J=17.8 Hz, C-p)*, 117.4 (t, J=17.7 Hz, C-s)*, 126.6 (C-a), 128.8 (aromatic carbon), 128.9 (aromatic carbon), 128.9 (C-c), 129.4 (aromatic carbon), 130.1 (aromatic carbon), 142.9 (d, J=246.6 Hz, C-q)**, 143.2 (d, J=241.3 Hz, C-r)**, 143.5 (C-e), 160.2 (C-g)***, 162.6 (C-h)***. MS (ESI) 436 (M+1). ESI-HRMS (m/z): [M+H]+ calcd for C₂₀H₁₁ClF₄N₃O₂, 436.0470; obsd, 436.0472.

General procedure for synthesis of pyrazolo[1,5-a]pyridine related amides (73-82). Oxalyl chloride (1.75 mL, 3.5 mmol) and dry DMF (7 μL) were added to a cooled (0° C.) solution of 45 (1.0 mmol) in dry THF (15 mL) under a nitrogen atmosphere. The reaction mixture was stirred for 2 hours at room temperature under a nitrogen atmosphere, then concentrated under reduced pressure. The residue was dissolved in dry THF (10 mL) and the solution again concentrated, this step was repeated three times. A solution of the appropriate aniline (83-92, 1.00 mmol), dry pyridine (3.0 mmol) in dry toluene (5 mL) was added dropwise to the solution of above acyl chloride in dry toluene (15 mL) kept under nitrogen atmosphere. The resulting mixture was stirred at reflux overnight, then cooled to room temperature and only for compounds 75-78 quenched with 0.5 M HCl (25 mL). The layers were resolved, the aqueous phase was further extracted with EtOAc (3×50 mL), the combined organic layers were washed with brine, dried over Na₂SO₄ and evaporated under reduced pressure. The crude material was purified using flash chromatography (details in each specific recipe).

2-Benzyloxy-N-(5-phenoxypiridin-2-yl)pyrazolo[1,5-a]pyridine-3-carboxamide (73). Obtained from 45 using aniline 83. Flash chromatography eluent: petroleum ether/EtOAc 80/20 v/v. White solid (m.p. 120.6-120.7° C.; from trituration with diisopropyl ether) Yield 87%. ¹H NMR (600 MHz, Chloroform-d): δ 5.55 (s, 2H, —OCH₂Ph), 6.86-6.92 (m, 2H, aromatic proton and H-b), 7.10 (d, 2H, J=7.9 Hz, aromatic protons), 7.17 (t, 1H, J=7.4 Hz, H-c), 7.34-7.42 (m, 4H, aromatic protons), 7.45 (t, 2H, J=7.3 Hz, aromatic protons), 7.53 (d, 2H, J=7.2 Hz, aromatic protons), 8.01 (d, 1H, J=2.6 Hz, aromatic proton), 8.25-8.30 (m, 2H, aromatic proton and H-d), 8.32 (d, 1H, J=6.8 Hz, H-a), 8.64 (s, 1H, —NH). ¹³C NMR (151 MHz, Chloroform-d): δ 72.4 (—OCH₂ Ph), 90.5 (C-f), 111.8, 113.1 (C-b), 118.8 (C-d), 120.7, 124.4, 127.9 (C-c), 128.3, 128.8 (C-a), 129.1, 129.2, 129.8, 131.4, 131.9, 135.6, 138.6, 143.0 (C-e), 155.0 (C-s)*, 159.6 (C-v)*, 161.4 (C-g)*, 162.3 (C-h)*. IR (KBr) v (cm⁻¹): 3373, 3100, 3044, 2925, 1947, 1663, 1636, 1534, 1473, 1365, 1296, 1249, 1207, 1120, 1005. MS (ESI) 435 (M−1).

2-Benzyloxy-N-5-[3-(trifluoromethyl)phenoxy]piridin-2-ylpyrazolo[1,5-a]pyridine-3-carboxamide (74). Obtained from 45 using aniline 84. Flash chromatography eluent: petroleum ether/EtOAc 70/30 v/v). White solid (m.p. 133.6-135.9° C.; from trituration with diisopropyl ether). Yield 50%. ¹H NMR (600 MHz, Chloroform-d): δ (s, 2H, —OCH₂ Ph), 6.89 (t, 1H, J=6.7 Hz, H-b), 7.14 (dd, 1H, J=8.2, 1.9 Hz, aromatic proton), 7.23 (s, 1H, aromatic proton), 7.33-7.46 (m, 7H, aromatic protons), 7.60 (d, 2H, J=7.3 Hz, aromatic protons), 8.12 (d, 1H, J=2.8 Hz, aromatic proton), 8.29 (d, 1H, J=8.9 Hz, H-d), 8.31 (d, 1H, J=6.9 Hz, H-a), 8.42 (d, 1H, J=9.0 Hz, aromatic proton), 9.40 (s, 1H, —NH), ¹³C-NMR (151 MHz, Chloroform-d): δ 72.0 (—OCH₂ Ph), 90.7 (C-f), 113.1 (C-b), 114.6 (q, J=3.8 Hz), 114.9, 118.8 (C-d), 119.8 (q, J=3.8 Hz), 120.8, 123.8 (q, J=272.0 Hz, —CF₃), 127.9, 128.0, 128.6, 128.8, 128.9, 129.7, 130.6, 132.5 (q, J=32.8 Hz), 135.9, 140.3, 143.2 (C-e), 148.3, 148.9 (C-s)*, 158.2 (C-v)*, 161.2 (C-g)*, 162.4 (C-h)*. IR (KBr) v (cm⁻¹): 3373, 3069, 2924, 2853, 1666, 1634, 1538, 1449, 1328, 1287, 1163, 1130, 1012. MS (ESI) 505 (M+1).

2-(Benzyloxy)-N-(2-methyl-4-phenoxyphenyl)pyrazolo[1,5-a]pyridine-3-carboxamide (75). Obtained from 45 using aniline 85. Flash chromatography (eluent petroleum ether/EtOAc 85/15 v/v). Light yellow solid (m.p. 186.1-190.9° C. from diisopropyl ether). Yield 87%. ¹H NMR (600 MHz, Chloroform-d): δ 1.83 (s, 3H, Ar—CH₃), 5.54 (s, 2H, —OCH₂Ph), 6.76 (d, J=2.2 Hz, 1H, H-t), 6.85-6.91 (m, 2H, aromatic protons), 6.96 (d, 2H, J=8.1, aromatic protons), 7.05 (t, 1H, J=7.3 Hz, H-b), 7.30 (t, 2H, J=7.8 Hz, aromatic protons), 7.34-7.45 (m, 4H, aromatic protons and H-c), 7.53 (d, 2H, J=6.3 Hz, aromatic protons), 8.20 (d, 1H, J=8.8 Hz, H-d), 8.29-8.37 (m, 2H, aromatic protons), 8.44 (s, 1H, —NH). ¹³C NMR (151 MHz, Chloroform-d): δ 17.6 (Ar—CH₃), 72.6 (—OCH₂ Ph), 91.1 (C-f), 112.9 (C-b), 117.5 (C-d), 118.3, 119.0, 121.1, 122.8, 123.0, 127.6, 128.6, 129.0, 129.2, 129.4, 129.5, 129.7, 132.7, 135.4, 143.1 (C-e), 152.6 (C-g)*, 158.1 (C-s)*, 161.2 (C-v)*, 162.3 (C-h)*; IR (KBr) v (cm⁻¹): 3391, 3308, 3040, 2922, 2737, 1963, 1882, 1660, 1590, 1588, 1362, 1334, 1219, 1151, 1130, 1101; MS (ESI) 450 (M+1).

2-(Benzyloxy)-N-(2-isopropyl-5-methyl-4-phenoxyphenyl)pyrazolo[1,5-a]pyridine-3-carboxamide (76). Obtained from 45 using aniline 86. Flash chromatography eluent: petroleum ether/EtOAc 70/30 v/v). White solid (m.p. 166.2-167.7° C.; from trituration with diisopropyl ether). Yield 54%. ¹H NMR (600 MHz, Chloroform-d): δ 0.90 (d, 6H, J=6.8 Hz, CH(CH₃)₂), 2.16 (s, 3H, Ar—CH₃), 2.72 (hept, 1H, J=6.7 Hz, —CH(CH₃)₂), (s, 2H, —OCH₂Ph), 6.81 (s, 1H, H-t), 6.84 (d, 2H, J=8.1 Hz, aromatic protons), 6.87 (t, 1H, J=6.9 Hz, H-b), 6.98 (t, 1H, J=7.3 Hz, aromatic proton), 7.22-7.28 (m, 2H, aromatic protons), 7.34-7.45 (m, 4H, aromatic protons), 7.52 (d, 2H, J=6.9 Hz, aromatic protons), 8.02 (s, 1H, H-q), 8.31 (d, 1H, J=6.8 Hz, H-a), 8.35 (d, 1H, J=8.8 Hz, H-d), 8.48 (s, 1H, —NH). ¹³C NMR (151 MHz, Chloroform-d): δ 16.1 (Ar—CH₃), 22.7 CH(CH₃)₂), 27.8 (—CH(CH₃)₂), 72.5 (—OCH₂Ph), 91.1 (C-f) 112.9 (C-b), 116.3, 117.9, 119.1 (C-d), 121.8, 126.4, 127.6 (C-c), 128.3, 128.6, 129.0 (C-a), 129.1, 129.2, 129.7, 131.5, 135.5, 138.4, 143.2 (C-e), 150.4 (C-s)*, 158.7 (C-v)*, 161.5 (C-h)**, 162.4 (C-g)**; IR (KBr) v (cm⁻¹): 3398, 3040, 2963, 1652, 1636, 1528, 1490, 1445, 1402, 1368, 1289, 1220, 1181, 1147, 1127, 1044, 993; MS (ESI) 492 (M+1).

2-(Benzyloxy)-N-(5-isopropyl-2-methyl-4-phenoxyphenyl)pyrazolo[1,5-a]pyridine-3-carboxamide (77). Obtained from 45 using aniline 87. Flash chromatography eluent: petroleum ether/EtOAc 60/40 v/v). White solid (m.p. 157.5-158.9° C.; from trituration with diisopropyl ether). Yield 92%. ¹H NMR (600 MHz, Chloroform-d): δ 1.24 (d, 6H, J=6.8 Hz, —CH(CH₃)₂), 1.77 (s, 3H, Ar—CH₃), 3.20 (hept, 1H, J=6.8 Hz, —CH(CH₃)₂), 5.54 (s, 2H, —OCH₂Ph), 6.64 (s, 1H, H-t), 6.85-6.91 (m, 3H, aromatic protons and H-b), 7.01 (t, 1H, J=7.3 Hz, H-c), 7.20-7.45 (m, 6H, aromatic protons), 7.52 (d, 2H, J=6.4 Hz, aromatic protons), 8.29 (s, 1H, H-q), 8.32 (d, 1H, J=6.8 Hz, H-a), 8.37 (d, 1H, J=8.8 Hz, H-d), 8.47 (s, 1H, —NH). ¹³C NMR (151 MHz Chloroform-d) δ 17.1 (—CH(CH₃)₂), 23.2 (—CH(CH₃)₂), 27.4 (Ar—CH₃) 72.6 (—OCH₂ Ph), 91.2 (C-f), 112.8 (C-b), 117.0, 119.1 (C-d), 120.2, 122.0, 122.1, 126.4, 127.5, 128.6, 128.9, 129.2, 129.4, 129.6, 133.6, 135.4, 138.8, 143.0 (C-e), 148.8 (C-s)*, 159.0 (C-v)*, 161.2 (C-g)**, 162.3 (C-h)**; IR (KBr) v (cm⁻¹): 3392, 3045, 2970, 1652, 1636, 1597, 1533, 1486, 1456, 1407, 1360, 1290, 1223, 1146, 1129, 1007, 911; MS (ESI) 492 (M+1).

2-(Benzyloxy)-N-(2-isopropyl-5-methyl-4-(4-(trifluoromethyl)phenoxy)phenyl)pyrazolo[1,5-a]pyridine-3-carboxamide (78). Obtained from 45, using aniline 88. Flash chromatography (eluent petroleum ether/EtOAc 85/15 v/v) to obtain a white solid (186.2-187.3° C. from diisopropyl ether). Yield 95%. ¹H NMR (600 MHz Chloroform-d): δ 0.91 (d, 6H, J=6.8 Hz, —CH(CH₃)₂), 2.14 (s, 3H, Ar—CH₃), 2.73 (hept, 1H, J=6.8 Hz, —CH(CH₃)₂), 5.56 (s, 2H, —OCH₂Ph), 6.82 (s, 1H, H-t), 6.87-6.91 (m, 3H, aromatic protons and H-b), 7.36-7.46 (m, 4H, aromatic protons), 7.49-7.56 (m, 4H, aromatic protons), 8.10 (s, 1H, H-q), 8.33 (d, 1H, J=6.9 Hz, H-a), 8.35 (d, 1H, J=8.8 Hz, H-d), 8.53 (s, 1H, —NH). ¹³C NMR (151 MHz, Chloroform-d): δ 16.0 (Ar—CH₃), 22.7 (—CH(CH₃)₂), 27.8 (—CH(CH₃)₂), 72.6 (—OCH₂ Ph), 91.0 (C-f), 112.9 (C-b), 115.9, 118.2 (C-d), 119.0, 123.8 (q, J=33.2 Hz), 124.5 (q, J=270.9, —CF 3), 126.4, 127.2 (q, J=3.7 Hz), 127.7, 128.4, 128.7, 129.0, 129.2, 129.3, 132.3, 135.5, 138.5, 143.2 (C-e), 149.2 (C-s), 161.4 (C-v)*, 161.5 (C-g)*, 162.4 (C-h)*; IR (KBr) v (cm⁻¹): 3403, 3084, 3043, 2976, 2891, 1655, 1638, 1613, 1578, 1543, 1511, 1501, 1477, 1446, 1401, 1360, 1330, 12090, 1240, 1215, 1148, 1120, 1064, 1042, 994; MS (ESI) 560 (M+1).

2-(Benzyloxy)-N-(1H-indol-5-yl)pyrazolo[1,5-a]pyridine-3-carboxamide (79). Obtained from 45 using aniline 89. Flash chromatography eluent: petroleum ether/EtOAc 60/40 v/v). White solid (m.p. 133.6-135.9° C.; from trituration with diisopropyl ether). Yield 74%. ¹H NMR (600 MHz, DMSO-d₆): δ 5.60 (s, 2H, —OCH₂ Ph), 6.38 (s, 1H, aromatic proton), 7.03 (t, 1H, J=6.9 Hz, H-b), 7.11 (dd, 1H, J=8.6, 1.6 Hz, aromatic proton), 7.30-7.34 (m, 2H, aromatic protons), 7.41 (t, 1H, J=7.4 Hz, H-c), 7.47 (t, 2H, J=7.5 Hz, aromatic protons), 7.50-7.54 (m, 2H, aromatic protons), 7.65 (d, 2H, J=7.4 Hz, aromatic protons), 7.88 (s, 1H, aromatic proton), 8.13 (d, 1H, J=8.8 Hz, H-d), 8.69 (d, 1H, J=6.8 Hz, H-a), 8.82 (s, 1H, —NH), 11.03 (s, 1H, —NH indole). ¹³C-NMR (151 MHz, DMSO-d₆): δ 71.5 (—OCH₂ Ph), 90.4 (C-f), 101.1, 110.6, 111.4, 113.1 (C-b), 114.8, 117.5 (C-d), 126.1, 127.6, 128.1, 128.2, 128.5, 128.7, 129.4, 130.6, 132.8, 136.3, 141.9 (C-e), 160.0 (C-g)*, 161.6 (C-h)*. MS (ESI) 505 (M+1).

2-(Benzyloxy)-N-(1-phenyl-1H-indol-5-yl)pyrazolo[1,5-a]pyridine-3-carboxamide (80). Obtained from 45 using aniline 90. Flash chromatography eluent: petroleum ether/EtOAc 60/40 v/v). White solid (m.p. 133.6-135.9° C.; from trituration with diisopropyl ether) Yield 74%. ¹H NMR (600 MHz, DMSO-d₆): δ 5.60 (s, 2H, —OCH₂Ph), 6.38 (s, 1H, aromatic proton), 7.03 (t, 1H, J=6.9 Hz, H-b), 7.11 (dd, 1H, J=8.6, 1.6 Hz, aromatic proton), 7.30-7.34 (m, 2H, aromatic protons), 7.41 (t, 1H, J=7.4 Hz, H-c), 7.47 (t, 2H, J=7.5 Hz, aromatic protons), 7.50-7.54 (m, 2H, aromatic protons), 7.65 (d, 2H, J=7.4 Hz, aromatic protons), 7.88 (s, 1H, aromatic proton), 8.13 (d, 1H, J=8.8 Hz, H-d), 8.69 (d, 1H, J=6.8 Hz, H-a), 8.82 (s, 1H, —NH). ¹³C-NMR (151 MHz, DMSO-d₆): δ 71.5 (—OCH₂Ph), 90.4 (C-f), 101.1, 110.6, 111.4, 113.1 (C-b), 114.8, 117.5 (C-d), 126.1, 127.6, 128.1, 128.2, 128.5, 128.7, 129.4, 130.6, 132.8, 136.3, 141.9 (C-e), 160.0 (C-g)*, 161.6 (C-h)*. MS (ESI) 505 (M+1).

2-(Benzyloxy)-N-(1-phenyl-1H-benzo[d]imidazol-5-yl)pyrazolo[1,5-a]pyridine-3-carboxamide (81). Obtained from 45 using aniline 91. Flash chromatography eluent: DCM/MeOH 95/5 v/v). Pale pink solid (m.p. 133.6-135.9° C.; from trituration with diisopropyl ether) Yield 67%. ¹H NMR (600 MHz, Chloroform-d): δ 5.29 (s, 1H, aromatic proton), 5.59 (s, 2H, —OCH₂Ph), 6.87 (t, 1H, J=6.8 Hz, H-b), 7.35-7.60 (m, 11H, aromatic protons), 7.63 (d, 1H, J=8.7 Hz, aromatic proton), 7.94 (s, 1H, aromatic proton), 8.10 (s, 1H, aromatic proton), 8.31 (d, 1H, J=6.8 Hz, H-a), 8.35 (d, 1H, J=8.8 Hz, H-d), 8.84 (s, 1H, —NH). ¹³C-NMR (151 MHz, Chloroform-d): δ 72.3 (—OCH₂ Ph), 91.1 (C-f), 110.5, 111.4, 112.9 (C-b), 117.6, 119.0 (C-d), 124.0, 127.6, 128.1, 128.3, 128.7, 129.0, 129.1, 130.2, 130.4, 134.5, 135.9, 136.5, 142.9, 143.0 (C-e), 144.5, 161.4 (C-g)*, 162.3 (C-h)*. MS (ESI) 505 (M+1).

2-(Benzyloxy)-N-(1-phenyl-1H-benzo[d][1,2,3]triazol-5-yl)pyrazolo[1,5-a]pyridine-3-carboxamide (82). Obtained from 45 using aniline 92. Flash chromatography eluent: DCM/MeOH 95/5 v/v). Pale pink solid (m.p. 133.6-135.9° C.; from trituration with diisopropyl ether) Yield %. ¹H NMR (600 MHz, Chloroform-d): δ 5.61 (s, 2H, —OCH₂Ph), 7.06 (t, 1H, J=6.8 Hz, H-b), 7.40 (t, 1H, J=7.3 Hz, H-c), 7.47 (t, 2H, J=7.5 Hz, aromatic protons), 7.53-7.72 (m, 7H, aromatic protons), 7.85-7.92 (m, 3H, aromatic protons), 8.12 (d, 1H, J=8.8 Hz, H-d), 8.59 (s, 1H, aromatic proton), 8.70 (d, 1H, J=6.8 Hz, H-a), 9.23 (s, 1H, —NH). ¹³C-NMR (151 MHz, Chloroform-d): δ 71.5 (—OCH₂ Ph), 90.0 (C-f), 107.6, 111.3 (C-b), 113.5, 117.4 (C-d), 122.5, 128.1, 128.4, 128.5, 128.6, 128.7, 128.8, 129.6, 130.1, 135.8, 136.3, 136.4, 142.0 (C-e), 146.3, 160.6 (C-g)*, 161.9 (C-h)*. MS (ESI) 505 (M+1).

General hydrogenation procedure for target compounds 19-24, 28-30, and 37. 10% Palladium on carbon (Pd/C, mg) was added to a solution of the appropriate amide (compounds 73-82, 0.300 mmol) in dry THF (15 mL). The resulting mixture was vigorously stirred under a hydrogen atmosphere for 3 hours. The suspension was filtered through Celite and the cake was washed with methanol. The filtrate was concentrated under reduced pressure. When necessary, the obtained solid was further purified by flash chromatography.

2-Hydroxy-N-(1H-indol-5-yl)pyrazolo[1,5-a]pyridine-3-carboxamide (19). Obtained from 79. Flash chromatography eluent: dichloromethane/methanol 95/5 v/v). White solid (m.p. 133.6-135.9° C.; from trituration with diisopropyl ether). Yield 74%. ¹H NMR (600 MHz, DMSO-d₆): δ 6.39 (s, 1H, aromatic proton), 6.95 (t, 1H, J=6.7 Hz, H-b), 7.24 (dd, 1H, J=8.6, 1.1 Hz, aromatic proton), 7.27-7.38 (m, 2H, aromatic protons), 7.44 (t, 1H, J=7.8 Hz, H-c), 7.97 (d, 1H, J=1.9 Hz, aromatic proton), 8.07 (d, 1H, J=8.8 Hz, H-d), 8.55 (d, 1H, J=6.8 Hz, H-a), 9.03 (s, 1H, —NH), 11.02 (s, 1H, —NH indole), 18.84 (v br s, 1H, —OH). ¹³C-NMR (151 MHz, DMSO-d₆): δ 89.6 (C-f), 101.1, 110.6, 111.4, 112.5 (C-b), 115.0, 117.1 (C-d), 126.0, 127.3 (C-c), 127.7, 128.3, 130.8 (C-a), 132.7, 141.4 (C-e), 160.8 (C-g)*, 162.1 (C-h)*. MS (ESI) 293 (M+1).

2-Hydroxy-N-(1-phenyl-1H-indol-5-yl)pyrazolo[1,5-a]pyridine-3-carboxamide (20). Obtained from 80. Flash chromatography eluent: DCM/methanol 95/5 v/v). White solid (m.p. 133.6-135.9° C.; from trituration with diisopropyl ether). Yield 90%. ¹H NMR (600 MHz, DMSO-d₆): δ 6.68 (d, 1H, J=3.0 Hz, aromatic proton), 6.96 (t, 1H, J=6.7 Hz, H-b), 7.32-7.42 (m, 2H, aromatic protons), 7.45 (t, 1H, J=7.8 Hz, H-c), 7.49-7.62 (m, 5H, aromatic protons), 7.65 (d, 1H, J=3.1 Hz, aromatic proton), 8.08 (d, 1H, J=8.8 Hz, H-d), 8.13 (s, 1H, aromatic proton), 8.56 (d, 1H, J=6.7 Hz, H-a), 9.15 (s, 1H, —NH), 12.95 (s, 1H, —OH). ¹³C-NMR (151 MHz, DMSO-d₆): δ 89.6 (C-f), 103.6, 110.5, 111.2, 112.6 (C-b), 115.9, 117.0 (C-d), 123.5, 126.3, 127.4 (C-c), 128.9, 129.0, 129.3 (C-a), 129.9, 131.7, 132.2, 139.2, 141.4 (C-e), 160.9, 162.2. MS (ESI) 369 (M+1).

2-Hydroxy-N-(1-phenyl-1H-benzo[d]imidazol-5-yl)pyrazolo[1,5-a]pyridine-3-carboxamide (21). Obtained from 81. Flash chromatography eluent: DCM/MeOH 80/20 v/v). Pale pink solid (m.p. 133.6-135.9° C.; from trituration with diisopropyl ether). Yield 90%. ¹H NMR (600 MHz, DMSO-d₆): δ 6.87 (t, 1H, J=6.5 Hz, H-b), 7.37 (t, 1H, J=7.7 Hz, H-c), 7.46-7.54 (m, 2H, aromatic protons), 7.57 (d, 1H, J=8.6 Hz, aromatic proton), 7.63 (t, 2H, J=7.8 Hz, aromatic protons), 7.69 (d, 2H, J=7.7 Hz, aromatic protons), 8.00 (d, 1H, J=8.6 Hz, H-d), 8.32 (s, 1H, aromatic proton), 8.48 (d, 1H, J=6.4 Hz, H-a), 8.54 (s, 1H, aromatic proton), 9.99 (s, 1H, —NH). ¹³C-NMR (151 MHz, DMSO-d₆): δ 89.6 (C-f), 109.8, 110.6, 111.8, 116.4 (C-b), 116.5, 123.4, 126.7, 127.6 (C-c), 128.4, 128.9, 130.1 134.8, 136.1, 141.4 (C-e), 141.5, 143.6, 144.2, 161.9. MS (ESI) 368 (M−1).

2-Hydroxy-N-(1-phenyl-1H-benzo[d][1,2,3]triazol-5-yl)pyrazolo[1,5-a]pyridine-3-carboxamide (22). Obtained from 82. Flash chromatography eluent: DCM/MeOH 95/5 v/v). Grey solid (m.p. 133.6-135.9° C.; from trituration with diisopropyl ether). Yield 72%. ¹H NMR (600 MHz, DMSO-d₆): δ 6.85 (t, 1H, J=6.6 Hz, H-b), 7.35 (t, 1H, J=7.7 Hz, H-c), 7.58 (t, 1H, J=7.5 Hz, aromatic proton), 7.70 (t, 2H, J=7.9 Hz, aromatic protons), 7.78 (d, 1H, J=8.1 Hz, aromatic proton), 7.84-7.91 (m, 3H, aromatic protons), 7.95 (d, 1H, J=8.6 Hz, H-d), 8.44 (d, 1H, J=6.6 Hz, H-a), 8.71 (s, 1H, aromatic proton), 10.53 (s, 1H, —NH). ¹³C-NMR (151 MHz, DMSO-d₆): δ 89.5 (C-f), 106.8, 111.1 (C-b), 111.7, 116.1 (C-d), 122.5, 122.7, 126.6, 128.0, 128.3, 128.7, 130.1, 136.5, 136.8, 137.6, 141.5 (C-e), 146.5, 162.5. MS (ESI) 369 (M−1).

2-Hydroxy-N-(5-phenoxypiridin-2-yl)pyrazolo[1,5-a]pyridine-3-carboxamide (23). Obtained from 73, flash chromatography (eluent: DCM/methanol 90/10 v/v). White solid (m.p. 161.1-161.9° C. dec.; from trituration with diisopropyl ether). Yield 76%. ¹H NMR (600 MHz, DMSO-d₆): δ 6.75 (t, 1H, J=6.7 Hz, H-b), 6.97 (d, 1H, J=8.7 Hz, aromatic proton), 7.08 (d, 2H, J=8.0 Hz, aromatic protons), 7.16 (t, 1H, J=7.4 Hz, aromatic proton), 7.26 (t, 1H, J=7.8 Hz, aromatic proton) 7.39 (t, 2H, J=7.8 Hz, aromatic protons), 7.83 (d, 1H, J=8.7 Hz, H-d), 8.21 (dd, 1H, J=8.8, 2.5 Hz, aromatic proton), 8.33 (d, 1H, J=6.6 Hz, H-a), 8.48 (d, 1H, J=2.2 Hz, aromatic proton), 10.78 (s, 1H, —NH). ¹³C NMR (151 MHz, DMSO-d₆): δ 89.3 (C-f), 111.1 (C-b), 111.7, 115.6 (C-d), 120.2, 123.9, 125.9 (C-c), 127.9, 129.7, 131.1 (C-a), 132.9, 137.8, 141.4 (C-e), 154.9, 157.6 (C-g), 162.8 (C-h). MS (ESI) 345 (M−1). IR (KBr) v (cm⁻¹): 3061, 1653, 1636, 1534, 1476, 1379, 1248, 1205, 1124, 1023.

2-Hydroxy-N-5-[3-(trifluoromethyl)phenoxy]piridin-2-ylpyrazolo[1,5-a]pyridine-3-carboxamide (24). Obtained from 74, flash chromatography (eluent: DCM/methanol 90/10 v/v). White solid (m.p. 241.2-242.0° C. dec.; from trituration with diisopropyl ether). Yield 45%. ¹H NMR (600 MHz, DMSO-d₆): δ 6.59 (t, 1H, J=6.7 Hz, H-b), 7.10 (t, 1H, J=7.7 Hz, H-c), 7.26-7.34 (m, 2H, aromatic protons), 7.46 (d, 1H, J=7.6 Hz, aromatic proton), 7.55 (dd, 1H, J=9.0, 2.8 Hz, aromatic proton), 7.61 (t, 1H, J=8.0 Hz, aromatic proton), 7.65 (d, 1H, J=8.4 Hz, aromatic proton), 8.12 (d, 1H, J=2.8 Hz, aromatic proton), 8.14 (d, 1H, J=6.5 Hz, H-a), 8.40 (d, 1H, J=9.0 Hz, H-d), 12.48 (s, 1H, —NH); n C NMR (151 MHz, DMSO-d₆): δ 89.0 (C-f), 109.8, 113.6 (q, J=4.0 Hz), 114.3, 119.4 (q, J=3.4 Hz), 120.9, 123.8 (q, J=272.0 Hz, —CF₃), 124.4, 127.0, 129.9, 130.7 (q, J=32.1 Hz), 131.5, 140.1, 140.8, 141.7, 146.0, 150.7, 158.4, 163.9, 173.1. MS (ESI) 413 (M−1). IR (KBr) v (cm⁻¹): 3328, 2925, 1653, 1636, 1559, 1448, 1328, 1284, 1241, 1173, 1129, 1065.

2-Hydroxy-N-(2-methyl-4-(p-tolyloxy)phenyl) pyrazolo[1,5-a]pyridine-3-carboxamide (28). Obtained from 75, flash chromatography (eluent: DCM/methanol 90/10 v/v). White solid (m.p. 238.3-239.9° C. from trituration with diisopropyl ether). Yield 87%. ¹H NMR (600 MHz DMSO-d₆): δ 2.28 (s, 3H, Ar—CH₃), 6.88 (dd, 1H, J=8.8, 2.6 Hz, H-r), 6.94-7.01 (m, 4H, aromatic protons), 7.09 (t, 1H, J=7.3 Hz, H-b), 7.37 (t, 2H, J=7.9 Hz, aromatic protons), 7.47 (t, 1H, J=7.9, H-c), 8.06 (d, 1H, J=8.8 Hz, H-q), 8.20 (d, 1H, J=8.8 Hz, H-d), 8.57 (d, 1H, J=6.8 Hz, H-a), 8.93 (s, 1H, —NH), 12.99 (v br s, 1H, —OH). ¹³C NMR (151 MHz, DMSO-d₆): δ 17.6 (Ar—CH₃), 89.5 (C-f), 112.9 (C-b), 117.0, 117.1 (C-d), 117.9, 121.0, 122.2, 122.9, 127.8 (C-c), 129.0 (C-a), 129.2, 130.0, 133.1, 141.5 (C-e), 151.6 (C-s), 157.5 (C-v), 160.8 (C-g)*, 162.1 (C-h)*; IR (KBr) v (cm⁻¹): 3388, 3039, 2567, 1664, 1633, 1590, 1549, 1485, 1445, 1413, 1380, 1333, 1307, 1273, 1245, 1227, 1173, 1134; MS (ESI) 360 (M+1).

2-Hydroxy-N-(2-isopropyl-5-methyl-4-phenoxyphenyl)pyrazolo[1,5-a]pyridine-3-carboxamide (29). Obtained from 76, flash chromatography (eluent: DCM/methanol 98/2 v/v). White solid (m.p. 244.2-247.9° C. dec.; from trituration with diisopropyl ether). Yield 91%. ¹H NMR (600 MHz, DMSO-d₆): δ 1.17 (d, 6H, J=6.8 Hz, —CH(CH₃)₂), 2.10 (s, 3H, Ar—CH₃), 3.10 (hept, 1H, J=6.8 Hz, —CH(CH₃)₂), 6.85 (d, 2H, J=8.1 Hz, aromatic protons), 6.89 (s, 1H, H-t), 6.98 (t, 1H, J=6.9 Hz, H-b), 7.03 (t, 1H, J=7.3 Hz, aromatic proton), 7.33 (t, 2H, J=7.9 Hz, aromatic protons), 7.47 (t, 1H, J=7.9 Hz, H-c), 8.00 (s, 1H, H-q), 8.06 (d, 1H, J=8.8 Hz, H-d), 8.57 (d, 1H, J=6.8 Hz, H-a), 8.98 (s, 1H, —NH), 12.95 (v br s, 1H, —OH). ¹³C NMR (151 MHz, DMSO-d₆): δ (Ar—CH₃), 22.6 (—CH(CH₃)₂), 27.4 (—CH(CH₃)₂), 89.4 (C-f), 112.9 (C-b), 116.0, 117.1 (C-d), 117.5, 122.0, 125.7, 127.0, 127.8 (C-c), 129.0 (C-a), 129.9, 131.9, 138.0, 141.5 (C-e), 149.5, 158.0 (C-v), 161.0 (C-h)*, 162.1 (C-g)*. IR (KBr) v (cm⁻¹): 3400, 2964, 2579, 1661, 1637, 1547, 1492, 1446, 1404, 1332, 1228, 1185, 1130, 887; MS (ESI) 402 (M+1).

2-Hydroxy-N-(5-isopropyl-2-methyl-4-phenoxyphenyl)pyrazolo[1,5-a]pyridine-3-carboxamide (30). Obtained from 77, flash chromatography (eluent: DCM/methanol 95/5 v/v). White solid (m.p. 273.9-276.5° C. dec.; from trituration with diisopropyl ether). Yield 70%. ¹H NMR (600 MHz, DMSO-d₆) δ 1.16 (d, 6H, J=6.8 Hz, —CH(CH₃)₂), 2.23 (s, 3H, Ar—CH₃), 3.09 (hept, 1H, J=6.8 Hz, —CH(CH₃)₂), 6.82 (s, 1H, H-t), 6.87 (d, 2H, J=8.0 Hz, aromatic protons), 7.98 (t, 1H, J=6.6 Hz, H-b), 7.03 (t, 1H, J=7.2 Hz, aromatic proton), 7.33 (t, 2H, J=7.7 Hz, aromatic protons), 7.47 (t, 1H, J=7.8 Hz, H-c), 8.10 (d, 1H, J=8.7 Hz, H-d), 8.31 (s, 1H, H-q), 8.58 (d, 1H, J=6.6 Hz, H-a), 8.98 (s, 1H, —NH), 13.01 (v br s, 1H, —OH). ¹³C NMR (151 MHz, DMSO-d₆): δ 17.0 (—CH(CH₃)₂), 23.0 (—CH(CH₃)₂), 26.6 (Ar—CH₃), 89.5 (C-f), 112.9 8 (C-b), 116.4, 117.1 (C-d), 119.1, 122.1, 122.2, 126.2, 127.7 (C-c), 129.0 (C-a), 129.9, 134.2, 137.6, 141.5 (C-e), 147.5, 158.5, 160.8 (C-g)*, 162.1 (C-h)*. IR (KBr) v (cm⁻¹): 3393, 2961, 2578, 1659, 1636, 1548, 1486, 1446, 1407, 1333, 1217, 1160, 1126, 1042, 978; MS (ESI) 402 (M+1).

2-Hydroxy-N-(2-isopropyl-5-methyl-4-(4-(trifluoromethyl)phenoxy)phenyl)pyrazolo[1,5-a]pyridine-3-carboxamide (37). Obtained from 78, flash chromatography (eluent: DCM/methanol 95/5 v/v). White solid (m.p. 249.2-249.9° C. from trituration with diisopropyl ether). Yield 98%. ¹H NMR (600 MHz, DMSO-d₆): δ 1.20 (d, 6H, J=6.7 Hz, —CH(CH₃)₂), 2.08 (s, 3H, Ar—CH₃), 3.08-3.16 (m, 1H, —CH(CH₃)₂), 6.95-7.03 (m, 4H, aromatic protons, H-t and H-b), 7.48 (t, 1H J=7.8 Hz, H-c), 7.70 (d, 2H J=8.6 Hz, aromatic protons), 8.07 (d, 1H, J=8.8 Hz, H-d), 8.10 (s, 1H, H-q), 8.58 (d, 1H, J=6.8 Hz, H-a), 9.05 (s, 1H, —NH), 13.01 (v br s, 1H, —OH). ¹³C NMR (151 MHz, DMSO-d₆): δ 15.6 (Ar—CH₃), 22.6 (CH(CH₃)₂), 27.4 (CH(CH₃)₂), 89.4 (C-f), 112.9 (C-b), 115.9, 117.0 (C-d), 118.2, 122.4 (q, J=33.1 Hz), 124.4 (q, J=271.1 Hz, —CF 3), 125.5, 127.1, 127.5 (q, J=3.7 Hz), 127.8 (C-c), 129.0 (C-a), 132.8, 138.1, 141.6 (C-e), 148.1 (C-s), 161.0 (C-v), 161.1 (C-g), 162.1 (C-h); IR (KBr) ν (cm⁻¹): 3402, 2948, 2576, 1665, 1640, 1615, 1550, 1515, 1482, 1446, 1404, 1334, 1250, 1214, 1183, 1156, 114, 1103; MS (ESI) 470 (M+1).

N-(2,5-dimethyl-4-(pyridin-4-ylthio)phenyl)-2-((4-methoxybenzyl)oxy)pyrazolo[1,5-a]pyridine-3-carboxamide (93). Oxalyl chloride (201 μL, 2.35 mmol, 3.6 eq.) and dry DMF (7 μL) were added to a cooled (0° C.) solution of 57 (0.783 mmol, 1.2 eq.) in dry THF (15 mL), under a nitrogen atmosphere. The reaction mixture was stirred for 2 hours at room temperature under a nitrogen atmosphere. The solution was concentrated under reduced pressure and the residue was dissolved in dry THF (10 mL), this step was repeated three times. A solution of aniline 100 (0.652 mmol, 1 eq.) and dry pyridine (2.347 mmol, 3.6 eq.) in dry toluene (5 mL) was added to the solution of acyl chloride under nitrogen atmosphere. Due to the partial insolubility of 100 in dry toluene, 5 mL of dry THF were added. The resulting mixture was stirred at room temperature for 12 h then at 70° C. overnight. The mixture was then quenched with 0.5M HCl (25 mL). The layers were resolved, the aqueous phase was further extracted with EtOAc (3×50 mL), the combined organic layer was dried and evaporated under reduced pressure. The crude material was purified using flash chromatography (eluent: from petroleum ether/EtOAc 70:30 v/v to DCM/methanol 80:20 v/v) to afford the title compound as a brown solid (melting point: missing). Yield 80%. ¹H-NMR (600 MHz, Chloroform-d): δ 1.77 (s, 3H, Ar—CH₃), 2.34 (s, 3H, Ar—CH₃), 3.84 (s, 3H, —OCH₃), 5.48 (s, 2H, —OCH₂Ph), 6.81 (d, 2H, J=5.7 Hz, aromatic proton), 6.90 (t, 1H, J=6.9 Hz, H-b), 6.94 (d, 2H, J=8.4 Hz, H-n), 7.24 (s, 1H, aromatic protons), 7.40 (t, 1H, J=7.9 Hz, H-c), 7.47 (d, 2H, J=8.4 Hz, aromatic protons), 8.28 (d, 2H, J=5.5 Hz, aromatic protons); 8.31-8.36 (m, 2H, aromatic protons), 8.51 (s, 1H, aromatic proton), 8.62 (s, 1H, —NH); ¹³C-NMR (151 MHz Chloroform-d): δ 16.8 (Ar—CH₃), 20.7 (Ar—CH₃), 55.5 (—OCH₂ Ph), 72.6 (—OCH₂ Ph), 91.1 (C-f), 113.1 (C-b), 114.3, 118.9 (C-d), 120.1, 121.0, 122.5, 125.3, 127.4, 127.9 (C-a), 128.8 (C-c), 131.4, 138.5, 139.4, 141.9, 143.1 (C-e), 149.4, 150.9, 160.5, 161.4, 162.4; MS (ESI) 511 (M+1).

General procedure for synthesis of pyrazolo[1,5-a]pyridine related amides (94-99). Oxalyl chloride (1.75 mL, 1.5 mmol) and dry DMF (10 μL) were added to a cooled (0° C.) solution of 57 (1.2 mmol) in dry THF (15 mL), kept under a nitrogen atmosphere. The reaction mixture was stirred for 2 hours at room temperature under a nitrogen atmosphere. The solution was concentrated under reduced pressure and the residue was dissolved in dry THF (10 mL), this step was repeated three times. To a solution of the appropriate aniline (101-106, 1.00 mmol) and dry pyridine (3.6 mmol) in dry toluene (5 mL), was added to the solution of the above acyl chloride in dry toluene (10 mL), kept under nitrogen atmosphere. The resulting mixture was stirred at reflux overnight, then quenched with 0.5M HCl (25 mL). The layers were resolved, the aqueous phase was further extracted with ethyl acetate (3×50 mL), and the combined organic layer was dried and evaporated under reduced pressure. The crude material was purified using flash chromatography.

2-(Benzyloxy)-N-(2-isopropoxy-5-methyl-4-phenoxyphenyl)pyrazolo[1,5-a]pyridine-3-carboxamide (94). Obtained from 57, using aniline 101. The crude product was purified by flash chromatography (eluent: from petroleum ether/EtOAc 85:15 v/v) to afford the title compound as a sticky solid. Yield 90%. ¹H NMR (600 MHz, Chloroform-d): δ 1.12 (d, 6H, J=6.1 Hz, —CH(CH₃)₂), 2.17 (s, 3H, Ar—CH₃), 4.36 (h, 1H, J=6.1 Hz, —CH(CH₃)₂), 5.67 (s, 2H, —OCH₂Ph), 6.55 (s, 1H, H-t), 6.83 (t, 1H, J=6.8 Hz, H-b), 6.88 (d, 2H, J=8.1 Hz, H-n), 7.0 (t, 1H, J=7.3 Hz, aromatic proton), 7.25-7.41 (m, 6H, aromatic protons), 7.53 (d, 2H, J=7.5 Hz, aromatic protons), 8.26 (d, 1H, J=6.8 Hz, H-a), 8.35 (d, 1H, J=8.9 Hz, H-d), 8.48 (s, 1H, H-q), 9.20 (s, 1H, —NH); ¹³C NMR (151 MHz, Chloroform-d): {δ 15.8 (Ar—CH₃), 21.9 (—CH(CH₃)₂), 71.5 (—CH(CH₃)₂), 71.6 (—OCH₂ Ph), 91.6 (C-f), 106.7, 112.7 (C-b), 116.2, 119.0 (C-d), 121.8, 122.5, 122.9, 126.3, 127.4, 127.9, 128.4, 128.6, 128.8, 129.7, 136.4, 143.2 (C-e), 145.7, 148.6, 158.8, 161.3, 162.3. MS (ESI) 538 (M−1).

N-(2-Cyclobutoxy-5-methyl-4-phenoxyphenyl)-2-((4-methoxybenzyl)oxy)pyrazolo[1,5-a]pyridine-3-carboxamide (95). Obtained from 57 using aniline 102. The crude product was purified by flash chromatography (eluent: petroleum ether/EtOAc 85:15 v/v) to afford the title compound as a sticky solid. Yield 79%. ¹H NMR (600 MHz, Chloroform-d): δ 1.46-1.56 (m, 1H, cyclobutoxy proton), 1.59-1.68 (m, 1H, cyclobutoxy proton), 1.80-1.91 (m, 2H, cyclobutoxy proton), 2.15 (s, 3H, Ar—CH₃), 2.18-2.25 (m, 2H, cyclobutoxy proton), 3.80 (s, 3H, —OCH₃), 4.46 (p, 1H, J=7.1 Hz, cyclobutoxy proton), 5.58 (s 2H, —OCH₂Ph), 6.38 (s, 1H, H-t), 6.84 (t, 1H, J=6.9 Hz, H-b), 6.87 (d, 2H, J=8.0 Hz, aromatic protons), 6.91 (d, 2H, J=8.4 Hz, H-n), 7.00 (t, 1H, J=7.3 Hz, aromatic protons), 7.25-7.30 (m, 2H, aromatic protons), 7.35 (t, 1H, J=7.9 Hz, H-c), 7.49 (d, 2H, J=8.4 Hz, H-m), 8.28 (d, 1H, J=6.8 Hz, H-a), 8.34 (d, 1H, J=8.8 Hz, H-d), 8.44 (s, 1H, H-q), 9.16 (s, 1H, —NH); ¹³C NMR (151 MHz, Chloroform-d): δ 13.1, 15.8, 30.5, 55.4, 71.6, 72.2 (—OCH₂ Ph), 91.6 (C-f), 105.6, 112.7 (C-b), 114.1, 116.3, 118.9 (C-d), 121.9, 122.3, 122.8, 125.3, 127.4, 128.4, 128.6, 129.7, 129.8, 143.1 (C-e), 145.4, 148.6, 158.6, 159.8, 161.3, 162.4. MS (ESI) 550 (M+1).

N-(2-(Sec-butoxy)-5-methyl-4-phenoxyphenyl)-2-((4-methoxybenzyl)oxy)pyrazolo[1,5-a]pyridine-3-carboxamide (96). Obtained from 57, using aniline 103. The crude product was purified by flash chromatography (eluent: petroleum ether/EtOAc 85:15 v/v) to afford the title compound as a sticky solid. Yield 94%. ¹H NMR (600 MHz, Chloroform-d): δ 0.85 (t, 3H, J=7.5 Hz, —CHCH₂CH₃), 1.12 (d, 3H, J=6.1 Hz, —CH₃CHCH₂CH₃), 1.33-1.60 (m, 2H, —CH₃CHCH₂CH₃), 2.16 (s, 3H, Ar—CH₃), 3.79 (s, 3H, —OCH₃), 4.12 (h, 1H, J=6.1 Hz, —CH₃CHCH₂CH₃), 5.56 (d, 1H, J=12.1 Hz, —OCH₂ Ph), 5.59 (d, 1H, J=12.1 Hz, —OCH₂Ph). 6.54 (s, 1H, H-t), 6.83 (t, 1H, J=6.8 Hz, H-b), 6.86-6.92 (m, 4H, aromatic protons), 7.0 (t, 1H, J=7.3 Hz, aromatic proton), 7.25-7.31 (m, 2H, aromatic protons), 7.34 (t, 1H, J=7.9 Hz, H-c), 7.47 (d, 2H, J=8.5 Hz, H-m), 8.27 (d, 1H, J=6.8 Hz, H-a), 8.34 (d, 1H, J=8.9 Hz, H-d), 8.46 (s, 1H, H-q), 9.17 (s, 1H, —NH); ¹³C NMR (151 MHz, Chloroform-d): δ 10.1 (—CH₃ CHCH₂ CH₃) 15.8 (Ar—CH₃), 19.3 (—CH₃CHCH₂CH₃), 29.1 (—CH₃CHCH₂CH₃), 55.4 (—OCH)) 71.5 (—CH₃CHCH₂CH₃), 76.8 (—OCH₂ Ph), 91.6 (C-f), 106.7, 112.6 (C-b), 114.1, 116.2, 119.0 (C-d), 121.8, 122.4, 123.0, 126.3, 127.3, 128.4, 128.6, 129.7, 129.9, 143.2 (C-e), 146.0, 148.6, 158.8, 159.8, 161.4, 162.3. MS (ESI) 552 (M+1).

2-((4-Methoxybenzyl)oxy)-N-(5-methyl-2-(pentan-2-yloxy)-4-phenoxyphenyl)pyrazolo[1,5-a]pyridine-3-carboxamide (97). Obtained from 57, using aniline 104. The crude product was purified by flash chromatography (eluent: petroleum ether/EtOAc 85:15 v/v) to afford the title compound as a sticky solid. Yield 89%. ¹H NMR (600 MHz, Chloroform-d): δ 0.82 (t, 3H, J=6.9 Hz, —CH₂ CH₂ CH₃), 1.11 (d, 2H, J=6.0 Hz, —CH₃ CHCH₂ CH₂ CH₃) 1.21-1.40 (m, 3H, —CH₃CHCH₂CH₂CH₃), 1.47-1.57 (m, 1H, —CH₃CHCH₂CH₂CH₃), 2.16 (s, 3H, Ar—CH₃), 3.79 (s, 3H, —OCH₃), 4.16-4.23 (m, 1H, —CH₃CHCH₂CH₂CH₃), 5.56 (d, 1H, J=12.1 Hz, —OCH₂Ph), 5.60 (d, 1H, J=12.1 Hz, —OCH₂ Ph), 6.54 (s, 1H, H-t), 6.83 (t, 1H, J=6.7 Hz, H-b), 6.85-6.92 (m, 4H, aromatic protons), 7.0 (t, 1H, J=7.3 Hz, aromatic proton), 7.24-7.31 (m, 2H, aromatic protons), 7.34 (t, 1H, J=7.9 Hz, H-c), 7.47 (d, 2H, J=8.5 Hz, H-m), 8.27 (d, 1H, J=6.8 Hz, H-a), 8.34 (d, 1H, J=8.8 Hz, H-d), 8.47 (s, 1H, H-q), 9.17 (s, 1H, —NH); ¹³C NMR (151 MHz, Chloroform-d): 814.1 (—CH₃ CHCH₂ CH₂ CH₃) 15.8 (Ar—CH₃), 18.9 (—CH₃CHCH₂CH₂CH₃), 19.8 (—CH₃CHCH₂CH₂CH₃), 38.4 (—CH₃CHCH₂CH₂CH₃), 55.4 (—OCH₃), 71.5 (—OCH₂ Ph), 75.3 (—CH₃ CHCH₂ CH₂ CH₃), 91.6 (C-f), 106.6, 112.6 (C-b), 114.1, 116.2, 118.9 (C-d), 121.8, 122.4, 122.9, 126.3, 127.3, 128.4, 128.6, 129.7, 129.8, 143.2 (C-e), 146.0, 148.6, 158.7, 159.8, 161.4, 162.3. MS (ESI) 566 (M+1).

2-((4-Methoxybenzyl)oxy)-N-(5-methyl-2-(pentan-3-yloxy)-4-phenoxyphenyl)pyrazolo[1,5-a]pyridine-3-carboxamide (98). Obtained from 57 using aniline 105. The crude product was purified by flash chromatography (eluent: petroleum ether/EtOAc 85:15 v/v) to afford the title compound as a sticky solid (melting point from XXX). Yield 61%. ¹H NMR (600 MHz, Chloroform-d): δ 0.84 (t, 6H, J=7.4 Hz, —CHCH₂ CH₃), 1.43-1.54 (m, 4H, —CHCH₂ CH₃), 2.15 (s, 3H, Ar—CH₃), 3.79 (s, 3H, —OCH₃), 3.97 (p, 1H, J=5.8 Hz, —CHCH₂CH₃), 5.58 (s, 2H, —OCH₂Ph), 6.54 (s, 1H, H-t), 6.83 (t, 1H, J=6.8 Hz, H-b), 6.85-6.92 (m, 4H, aromatic protons), 7.0 (t, 1H, J=7.3 Hz, aromatic proton), 7.25-7.31 (m, 2H, aromatic protons), 7.34 (t, 1H, J=7.9 Hz, H-c), 7.46 (d, 2H, J=8.5 Hz, H-m), 8.27 (d, 1H, J=6.8 Hz, H-a), 8.34 (d, 1H, J=8.9 Hz, H-d), 8.47 (s, 1H, H-q), 9.20 (s, 1H, —NH); ¹³C NMR (151 MHz, Chloroform-d): δ 9.8 (—CHCH₂ CH₃) 15.8 (Ar—CH₃), 26.3 (—CHCH₂CH₃), 55.4 (—OCH₃), 71.5 (—OCH₂Ph), 81.9 (—CHCH₂CH₃), 91.6 (C-f), 106.5, 112.6 (C-b), 114.1, 116.2, 119.0 (C-d), 121.8, 122.3, 122.9, 126.3, 127.3, 128.5, 128.6, 129.7, 129.8, 143.2 (C-e), 146.5, 148.5, 158.7, 159.8, 161.4, 162.3. MS (ESI) 566 (M+1).

N-(2-Isopropyl-5-methyl-4-(pyridin-4-yloxy)phenyl)-2-((4-methoxybenzyl)oxy)pyrazolo[1,5-a]pyridine-3-carboxamide (99). Oxalyl chloride (198 μL, 2.23 mmol, 3.6 eq.) and dry DMF (7 μL), were added to a cooled (0° C.), solution of 57 (0.743 mmol, 1.2 eq.), in dry THF (15 mL), under a nitrogen atmosphere. The reaction mixture was stirred for 2 hours at room temperature under a nitrogen atmosphere. The solution was concentrated under reduced pressure and the residue was dissolved in dry THF (10 mL, this step was repeated three times). The resulting acyl chloride was dissolved in dry toluene (10 mL). A solution of aniline 106 (0.619 mmol, 1 eq.), and dry pyridine (2.228 mmol, 3.6 eq.), in dry toluene (5 mL), was added to the solution of acyl chloride under nitrogen atmosphere. The resulting mixture was stirred at room temperature overnight. The mixture was quenched in water (80 mL) and partially concentrated under reduce pressure. The aqueous phase was extracted with EtOAc (3×50 mL). The combined organic layers were dried and evaporated under reduced pressure. The crude material was purified using flash chromatography (eluent: petroleum ether/EtOAc 50:50 v/v) to afford the title compound as a brown solid (melting point: missing). Yield 80%. ¹H NMR (600 MHz, Chloroform-d): δ 0.94 (d, 6H, J=6.7 Hz, CH(CH₃)₂), 2.11 (s, 3H, —CH₃), 2.70 (hept, 1H, J=6.7 Hz, —CH(CH₃)₂), 3.82 (s, 3H, —OCH₃), 5.49 (s, 2H, —OCH₂Ph), 6.74 (d, 2H, J=4.3 Hz, aromatic protons), 6.83 (s, 1H, aromatic proton), 6.88 (t, 1H, J=6.8 Hz, H-b), 6.95 (d, 2H, J=8.4 Hz, H-n), 7.38 (t, 1H, J=7.9 Hz, H-c), 7.47 (d 2H, J=8.4 Hz, H-m), 8.13 (s, 1H, aromatic proton), 8.31-8.36 (m, 2H, aromatic protons), 8.41 (d, 2H, J=4.7 Hz, aromatic protons), 8.55 (s, 1H, —NH). ¹³C NMR (151 MHz, Chloroform-d): δ 15.9 (Ar—CH₃), 22.6 (—CH(CH₃)₂), 27.8 (—CH(CH₃)₂), 55.5 (—OCH₃), 72.4 (—OCH₂Ph), 91.0 (C-f), 111.3, 112.9 (C-b), 114.3, 118.3, 119.0 (C-d), 126.1, 127.6, 127.7 (C-a), 128.3, 128.7 (C-c), 131.1, 132.9, 138.3, 143.2 (C-e), 148.1, 151.4, 160.4, 161.6, 162.5, 165.2. MS (ESI) 523 (M+1).

General procedure for synthesis of compounds 26, 31-36. Thioanisole (10.0 eq.) was added to a solution of the respective protected amide 93-99 (1.0 eq.) in TFA (4 mL). The mixture was heated at 70° C. for 4 h then cooled to r.t. The mixture was partially concentrated, the crude was taken up with phosphate saline buffer pH=5 obtaining a suspension that was filtered, the solid so obtained was triturated with hexane to afford the title compounds, in pure form.

2-Hydroxy-N-(2,5-dimethyl-4-(pyridin-4-ylthio)phenyl) pyrazolo[1,5-a]pyridine-3-carboxamide (26). White solid. Yield 80%. ¹H-NMR (600 MHz, DMSO-d₆): δ 2.31 (s, 3H, Ar—CH₃), 2.32 (s, 3H, Ar—CH₃), 7.02 (t, 1H, J=6.9 Hz, H-b), 7.28 (d, 2H, J=6.1 Hz, aromatic protons), 7.48-7.56 (m, 2H, aromatic protons), 8.08 (d, 1H, J=8.8 Hz, H-d); 8.50 (d, 2H, J=5.0 Hz, aromatic protons), 8.58-8.65 (m, 2H, aromatic protons), 9.20 (s, 1H, —NH); ¹³C-NMR (151 MHz DMSO-d₆): δ 16.6 (Ar—CH₃), 20.2 (Ar—CH₃). 89.4 (C-f), 113.2 (C-b), 117.0, 118.1 (C-d), 120.8, 121.7, 125.5, 128.2 (C-a), 129.1 (C-c), 138.1, 140.2, 140.9, 141.6, 143.8 (C-e), 160.9, 162.2. MS (ESI) 389 (M−1).

2-Hydroxy-N-(2-isopropoxy-5-methyl-4-phenoxyphenyl)pyrazolo[1,5-a]pyridine-3-carboxamide (31). White solid. Yield 89%. ¹H NMR (600 MHz, Chloroform-d): δ 1.40 (d, 6H, J=6.0 Hz, —CH(CH₃)₂), 2.19 (s, 3H, Ar—CH₃), 4.51 (h, 1H, J=6.0 Hz, —CH(CH₃)₂), 6.63 (s, 1H, H-t), 6.88-6.92 (m, 3H, H-b and aromatic protons), 7.0 (t, 1H, J=7.3 Hz, aromatic proton), 7.30 (t, 2H, J=7.9 Hz, aromatic protons), 7.43 (t, 1H, J=7.9 Hz, H-c), 8.27 (d, 1H, J=6.8 Hz, H-a), 8.35 (d, 1H, J=8.8 Hz, H-d), 8.52 (s, 1H, H-q), 9.32 (s, 1H, —NH); ¹³C NMR (151 MHz, Chloroform-d): δ 15.9 (Ar—CH₃), 22.4 (—CH(CH₃)₂), 72.0 (—CH(CH₃)₂), 91.1 (C-f), 107.3, 113.2 (C-b), 116.3, 118.8 (C-d), 122.0, 122.5, 123.1, 126.6, 127.4, 128.2, 129.8, 136.4, 141.9 (C-e), 145.4, 148.7, 158.7, 161.4, 162.8. MS (ESI) 418 (M−1).

N-(2-cyclobutoxy-5-methyl-4-phenoxyphenyl)-2-hydroxypyrazolo[1,5-a]pyridine-3-carboxamide (32). White solid. Yield 75%. ¹H NMR (600 MHz, DMSO-d₆): δ 1.55-1.65 (m, 1H, cyclobutoxy proton), 1.73-1.82 (m, 1H, cyclobutoxy proton), 2.00-2.14 (m, 2H, cyclobutoxy proton), 2.06 (s, 3H, Ar—CH₃), 2.30-2.39 (m, 2H, cyclobutoxy proton), 4.70 (p, 1H, J=7.0 Hz, cyclobutoxy proton), 6.53 (s, 1H, H-t), 6.85 (d, 2H, J=8.1 Hz, aromatic proton), 6.98 (t, 1H, J=6.8 Hz, H-b), 7.03 (t, 1H, J=7.3 Hz, aromatic proton), 7.32 (t, 2H, J=7.8 Hz, aromatic protons), 7.47 (t, 1H, J=7.8 Hz, H-c), 8.09 (d, 1H, J=8.7 Hz, H-d), 8.46 (s, 1H, H-q), 8.58 (d, 1H, J=6.7 Hz, H-a), 9.62 (s, 1H, —NH), 12.85 (br s, 1H, —OH); ¹³C NMR (151 MHz, DMSO-d₆): δ 12.7, 15.5, 29.8, 71.8, 89.7 (C-f), 105.9, 112.8 (C-b), 115.9, 117.0 (C-d), 120.8, 121.0, 122.0, 125.8, 127.7, 129.0, 129.9, 141.5 (C-e), 144.3, 147.4, 158.0, 160.6, 162.0. MS (ESI) 430 (M+1).

N-(2-(Sec-butoxy)-5-methyl-4-phenoxyphenyl)-2-hydroxypyrazolo[1,5-a]pyridine-3-carboxamide (33). White solid. Yield 76%. ¹H NMR (600 MHz, DMSO-d₆): δ 0.91 (t, 3H, J=7.4 Hz, —CHCH₂CH₃), 1.22 (d, 3H, J=6.0 Hz, —CH₃CHCH₂CH₃), 1.56-1.75 (m, 2H, —CH₃CHCH₂CH₃), 2.06 (s, 3H, Ar—CH₃), 4.36 (h, 1H, J=5.8 Hz, —CH₃CHCH₂CH₃), 6.73 (s, 1H, H-t), 6.85 (d, 2H, J=8.1 Hz, aromatic protons), 6.98 (t, 1H, J=6.8 Hz, H-b), 7.02 (t, 1H, J=7.3 Hz, aromatic proton), 7.32 (t, 2H, J=7.9 Hz, aromatic protons), 7.34 (m, 1H, H-c), 8.09 (d, 1H, J=8.8 Hz, H-d), 8.47 (s, 1H, H-q), 8.57 (d, 1H, J=6.8 Hz, H-a), 9.63 (s, 1H, —NH), 12.79 (br s, 1H, —OH); ¹³C NMR (151 MHz, DMSO-d₆): δ 9.4 (—CH₃ CHCH₂ CH₃) 15.5 (Ar—CH₃), 18.9 (—CH₃CHCH₂CH₃), 28.4 (—CH₃CHCH₂CH₃), 76.1 (—CH₃CHCH₂CH₃), 89.7 (C-f), 107.0, 112.8 (C-b), 115.8, 117.1 (C-d), 120.9, 121.1, 121.9, 126.8, 127.7, 129.0, 129.9, 141.5 (C-e), 144.9, 147.4, 158.1, 160.6, 162.0. MS (ESI) 432 (M+1).

2-Hydroxy-N-(5-methyl-2-(pentan-2-yloxy)-4-phenoxyphenyl) pyrazolo[1,5-a]pyridine-3-carboxamide (34). White solid. Yield 89%. ¹H NMR (600 MHz, DMSO-d₆): δ 0.84 (t, 3H, J=7.3 Hz, —CH₂CH₂CH₃), 1.22 (d, 2H, J=6.0 Hz, —CH₃CHCH₂CH₂CH₃), 1.27-1.44 (m, 2H, —CH₃CHCH₂CH₂CH₃), 1.47-1.57 (m, 1H, —CH₃CHCH₂CH₂CH₃), 1.63-1.74 (m, 1H, —CH₃CHCH₂CH₂CH₃), 2.06 (s, 3H, Ar—CH₃), 4.37-4.43 (m, 1H, —CH₃CHCH₂CH₂CH₃), 6.72 (s, 1H, H-t), 6.85 (d, 2H, J=8.1 Hz, aromatic protons), 6.96 (t, 1H, J=6.7 Hz, H-b), 7.02 (t, 1H, J=7.3 Hz, aromatic proton), 7.32 (t, 1H, J=7.8 Hz, aromatic proton), 7.45 (t, 1H, J=7.8 Hz, H-c), 8.08 (d, 1H, J=8.7 Hz, H-d), 8.47 (s, 1H, H-q), 8.55 (d, 1H, J=6.7 Hz, H-a), 9.71 (s, 1H, —NH), 12.84 (s, 1H, —OH); ¹³C NMR (151 MHz, DMSO-d₆): δ 13.9 (—CH₃CHCH₂CH₂CH₃) 15.5 (Ar—CH₃), 18.1 (—CH₃CHCH₂CH₂CH₃), 19.4 (—CH₃CHCH₂CH₂CH₃), 37.8 (—CH₃CHCH₂CH₂CH₃), 74.9 (—CH₃CHCH₂CH₂CH₃), 89.7 (C-f), 106.9, 112.6 (C-b), 115.8, 116.9 (C-d), 120.9, 121.0, 121.9, 126.8, 127.5, 128.9, 129.9, 141.5 (C-e), 144.9, 147.4, 158.1, 160.7 MS (ESI) 446 (M+1).

2-Hydroxy-N-(5-methyl-2-(pentan-3-yloxy)-4-phenoxyphenyl)pyrazolo[1,5-a]pyridine-3-carboxamide (35). White solid. Yield 30%. ¹H NMR (600 MHz, DMSO-d₆): δ 0.96 (t, 6H, J=7.4 Hz, —CHCH₂CH₃), 1.70-1.77 (m, 4H, —CHCH₂CH₃), 2.18 (s, 3H, Ar—CH₃), 4.12 (p, 1H, J=5.6 Hz, CHCH₂CH₃), 6.59 (s, 1H, H-t), 6.89 (d, 2H, J=8.1 Hz, aromatic protons), 6.93 (t, 1H, J=6.7 Hz, H-b), 7.02 (t, 1H, J=7.3 Hz, aromatic proton), 7.30 (t, 2H, J=7.8 Hz, aromatic protons), 7.47 (t, 1H, J=7.9 Hz, H-c), 8.27-8.41 (m, 2H, H-a and H-d), 8.49 (s, 1H, H-q), 9.25 (s, 1H, —NH); ¹³C NMR (151 MHz, DMSO-d₆): δ 9.7 (—CHCH₂CH₃) 15.9 (Ar—CH₃), 26.2 (—CHCH₂CH₃), 81.7 (—CHCH₂CH₃), 91.0 (C-f), 106.5, 113.4 (C-b), 116.3, 118.6 (C-d), 122.0, 122.5, 122.6, 126.1, 127.6, 128.6, 129.8, 141.7 (C-e), 146.0, 148.9, 153.2, 158.7, 161.4. MS (ESI) 446 (M+1).

2-Hydroxy-N-(2-isopropyl-5-methyl-4-(pyridin-4-yloxy)phenyl)pyrazolo[1,5-a]pyridine-3-carboxamide (36). White solid. Yield 74%. ¹H NMR (600 MHz, DMSO-d₆): δ 1.22 (d, 6H, J=6.8 Hz, CH(CH₃)₂), 2.01 (s, 3H, —CH₃), 3.14 (hept, 1H, J=6.8 Hz, —CH(CH₃)₂), 6.96-7.04 (m, 1H, H-b), 7.09-7.16 (m, 3H, aromatic protons), 7.49 (t, 1H, J=7.9 Hz, H-c), 8.07 (d, 1H, J=8.8 Hz, H-d), 8.18 (s, 1H, aromatic protons), 8.59 (d, 1H, J=6.8 Hz, H-a), 8.60-8.70 (m, 2H, aromatic protons), 9.10 (s, 1H, —NH). ¹³C NMR (151 MHz, DMSO-d₆): δ 15.4 (Ar—CH₃), 22.5 (—CH(CH₃)₂), 27.4 (—CH(CH₃)₂), 89.4 (C-f), 112.3, 112.9 (C-b), 117.0, 118.3 (C-d), 125.3 (C-a), 127.1, 127.9 (C-c), 129.0, 133.7, 138.1, 141.6, (C-e), 146.6, 146.7, 147.7, 161.0, 162.1. MS (ESI) 403 (M+1)

2-Hydroxypyrazolo[1,5-a]pyridine-3-carboxylic acid (107). 5M NaOH solution (5 eq.) was added to a solution of 44 (1 g, 4.84 mmol) in EtOH (20 mL), the reaction mixture was stirred at reflux for 6 h. Then the EtOH was evaporated under vacuum and the residue was taken up with distilled water (20 mL). The suspension was acidified with 6M HCl until pH=2 in order to obtain the product as precipitate. The white solid was filtered and washed with distilled water until neutrality. Yield: 98%.

Pyrazolo[1,5-a]pyridin-2-ol (108). A 6M NaOH solution (10 eq.) was added to a suspension of 2-hydroxypyrazolo[1,5-a]pyridine-3-carboxylic acid (107, 1.4 g, 7.86 mmol) in EtOH (25 mL). The reaction mixture was stirred and heated under reflux at 85° C. until compound 107 was completely dissolved, then 12M HCl (2.38 mL) was added dropwise at 85° C. The reaction mixture was stirred for 20 minutes. The reaction was quenched in distilled water (30 mL) and extracted with EtOAc (3×15 mL). Organic phases were collected, washed with a saturated NaHCO₃ solution (3×20 mL), a saturated brine solution, dried over Na₂SO₄ and concentrated under reduced pressure. The latter was filtered via flash chromatography (eluent: petroleum ether/ethyl acetate 9/1 v/v). Yield: 90%.

2,3,5,6-tetrafluoro-[1,1′-biphenyl]-4-amine (46). K₂CO₃ (0.33 g, 3.0 eq.) and Pd(PPh₃)₄ (0.187 g, 0.2 eq.) were added to a solution of 4-Bromo-2,3,5,6-tetrafluoroaniline (0.2 g, 0.81 mmol) in dioxane/H₂O (30 mL, 9/1 v/v) under inert atmosphere, and the reaction mixture was stirred for 1 h. Then phenylboronic acid (0.296 g, 3.0 eq.) was added and the reaction mixture was stirred at 90° C. overnight. The reaction was quenched in distilled water (30 mL) and extracted with EtOAc (3×15 mL). Organic layers were collected, washed with a saturated brine solution, dried over Na₂SO₄ and concentrated under reduced pressure. The latter was purified via flash chromatography (eluent: petroleum ether/ethyl acetate 95/5 v/v). Yield: 90%. ¹H NMR (300 MHz, Chloroform-d): δ 4.04 (s, 2H, —NH₂), 7.35-7.51 (m, 5H, aromatic protons); ¹³C NMR (75 MHz, Chloroform-d): δ 108.4 (t, J=17.1 Hz), 125.5 (tt, J=13.8, 3.6 Hz), 128.2 (t, J=2.2 Hz), 128.4, 128.6, 130.5 (t, J=2.0 Hz), 136.9 (d, J=238.4 Hz), 144.2 (d, J=242.0 Hz). MS (ESI) 242 (M+1).

(E)-3-((2,3,5,6-tetrafluoro-[1,1′-biphenyl]-4-yl)diazinyl)pyrazolo[1,5-a]pyridin-2-ol (25). A solution of NaNO₂ (51 mg, 0.74 mmol.) in water (5 mL) was added dropwise to a cooled (0° C.) solution of 2,3,5,6-tetrafluoro-[1,1′-biphenyl]-4-amine (46,150 mg, 0.62 mmol) in nitric acid (1.55 mL); the resulting mixture was stirred for 10 minutes. The resulting solution was added dropwise to a cooled solution of 108 (83 mg, 0.62 mmol) in water (5 mL) at pH-10. The resulting mixture was stirred for 5 minutes, and an orange precipitate was observed. The precipitate was filtrated over Büchner to afford the title compound as orange solid (triturated with diisopropyl ether). Yield 48%. ¹H NMR (600 MHz, DMSO-d₆): δ 7.27 (t, 1H, J=6.5 Hz, H-b), 7.50-7.60 (m, 5H, aromatic protons), 7.80 (t, 1H, J=7.7 Hz, H-c), 8.19 (d, 1H, J=8.2 Hz, H-d), 8.75 (d, 1H, J=6.6 Hz, H-a), 13C NMR (151 MHz, DMSO-d₆): δ 117.0, 117.2, 118.4, 127.4, 129.1, 129.3, 129.9, 130.7, 130.8, 130.9, 133.3, 133.4, 140.3 (d, J=246.5 Hz, CF), 144.2 (d, J=244.3 Hz, CF), 164.0; MS (ESI) 387 (M+1)

(E)-3-((4-phenoxyphenyl)diazinyl)pyrazolo[1,5-a]pyridin-2-ol (38). A solution of NaNO₂ (51 mg, 0.74 mmol, 1.2 eq.) in water (5 mL) was added dropwise to a cooled (0° C.) solution of 4-phenoxyaniline (46) (150 mg) in nitric acid (1.55 mL); the resulting mixture was stirred for 10 minutes. The resulting solution was added dropwise to a cooled solution of (108, 83 mg, 1 eq.) in water (5 mL) at pH-10. The resulting mixture was stirred for 5 minutes, and a precipitate was observed. The precipitate was filtrated over Büchner to afford the title compound as orange solid (triturated with diisopropyl ether). Yield 60%. ¹H NMR (600 MHz, DMSO-d₆): δ 7.04-7.13 (m, 4H, aromatic protons), 7.13-7.20 (m, 2H, aromatic proton, H-b), 7.42 (t, 2H, J=8.0 Hz, aromatic protons), 7.63 (t, 1H, J=7.8 Hz, H-c), 7.76 (d, 2H, J=8.8 Hz, aromatic protons), 8.15 (d, 1H, J=8.5 Hz, H-d), 8.61 (d, 1H, J=6.5 Hz, H-a), ¹³C NMR (151 MHz, DMSO-d₆): δ 116.2, 117.2, 117.7, 119.0, 119.0, 121.6, 123.8, 130.0 130.2, 130.7, 132.8, 145.5, 156.39, 156.42, 164.5. MS (ESI) 331 (M+1)

Ethyl 2-methyloxypyrazolo[1,5a]pyridine-3-carboxylate (110a) and ethyl N-methyl-2-oxo-pyrazolo[1,5a]pyridine-3-carboxylate (110b) from (44). Cs₂CO₃ (3 eq.) was added to a solution of 44 (5.0 g, 24.24 mmol) in dry DMF (50 mL), and the resulting mixture was stirred for 30 minutes at room temperature before adding iodomethane (29.1 mmol, 1.81 mL) drop-wise to the mixture. The reaction was quenched with distilled water (300 mL) and extracted with ethyl acetate (6×70 mL). Organic phases were collected, washed with brine solution, dried over Na₂SO₄, filtered and concentrated under reduced pressure to obtain a pale-yellow solid crude containing the two isomers. The latter were separated via flash chromatography (eluent: petroleum ether/ethyl acetate 6/4 v/v, then DCM/MeOH 95/5 v/v). Yield: 77% (110a) and 13% (110b).

(110a) m.p. 142.4-143.5° C.; ¹H NMR (600 MHz, Chloroform-d): δ 1.40 (t, 3H, J=7.1 Hz, —OCH₂CH₃), 4.12 (s, 3H—OCH₃), 4.37 (q, 2H, J=7.1 Hz, —OCH₂CH₃), 6.83 (t, 1H J=6.8 Hz, H-b), 7.35 (t, 1H, J=7.9 Hz, H-c), 7.97 (d, 1H, J=8.9 Hz, H-d), 8.28 (d, 1H, J=6.8 Hz, H-a). ¹³C NMR (151 MHz, Chloroform-d): δ 14.7 (—OCH₂CH₃), 57.0 (—OCH₃), 59.9 (—OCH₂CH₃), 88.1 (C-f), 112.6 (C-b), 118.4 (C-d), 127.9 (C-c)*, 128.9 (C-a)*, 142.9 (C-e), 163.3 (C-g)**, 166.0 (C-h)**. (ESI) 221 (M+1).

(110b) m.p. 234.4-235.8° C.; ¹H NMR (600 MHz, Chloroform-d): δ 1.28 (t, 3H, J=7.1 Hz, —OCH₂CH₃), 3.58 (s, 3H —OCH₃), 4.21 (q, 2H, J=7.1 Hz, —OCH₂C113), 7.10 (t, 1H J=6.9 Hz, H-b), 7.66 (t, 1H, J=7.9 Hz, H-c), 7.90 (d, 1H, J=8.9 Hz, H-d), 8.57 (d, 1H, J=6.8 Hz, H-a). ¹³C NMR (151 MHz, Chloroform-d): δ 15.5 (—OCH₂CH₃), 28.9 (—NCH₃), 59.3 (—OCH₂CH₃), 84.2 (C-f), 113.3 (C-b), 116.7 (C-d), 125.8 (C-c)*, 132.9 (C-a)*, 142.6 (C-e), 160.6 (C-g)**, 164.1 (C-h)**. (ESI) 221 (M+1).

2-Methoxypyrazolo [1,5-a]pyridine-3-carbohydrazide (111). 20 eq. of Hydrazine monohydrate (0.273 g, 20 eq.) was added to a solution of 110a (0.6 g, 2.73 mmol) in EtOH (3 mL) and the reaction mixture was stirred at reflux overnight. The reaction was cooled to room temperature and the solid was filtered and washed with distilled water to afford the title compound as white solid. Yield: 68.7%. ¹H NMR (600 MHz, Chloroform-d): δ 4.03 (s, 2H, —CONHNH₂), 4.13 (s, 3H, —OCH₃), 6.81 (t, 1H J=6.7 Hz, H-b), 7.32 (t, 1H, J=7.9 Hz, H-c), 7.87 (s, 1H, —CONHNH₂), 8.19 (d, 1H, J=8.9 Hz, H-d), 8.24 (d, 1H, J=6.9 Hz, H-a). ¹³C NMR (151 MHz, Chloroform-d): δ 57.2 (—OCH₃), 88.8 (C-f), 112.6 (C-b), 118.5 (C-d), 127.5 (C-c)*, 128.6 (C-a)*, 142.7 (C-e), 163.2 (C-g)*, 164.6 (C-h)*. (ESI) 207 (M+1).

2-(4-Bromo-2,3,5,6-tetrafluorophenyl)-5-(2-methoxypyrazolo [1,5-a]pyridin-3-yl)-1,3,4-oxadiazole (112). Compound 111 (0.3 g, 1.52 mmol) and 4-Bromo-2,3,5,6-tetrafluorobenzoic acid (0.414 g, 1.52 mmol) were dissolved in POCl₃ (5 mL). The reaction mixture was stirred at 50° C. for 4 h. The mixture was concentrated under vacuum and the solid was taken up with distilled water (80 mL) and extracted with EtOAc (3×50 mL). Organic layers were collected, washed with a saturated brine solution, dried over Na₂SO₄ and concentrated under reduced pressure to afford a pale, yellow crude. The latter was purified via flash chromatography (eluent: petroleum ether/ethyl acetate 7/3 v/v) to afford the title compound as pale-yellow solid. Yield: 520. ¹H NMR (600 MHz, Chloroform-d): δ 4.21 (s, 3H, —OCH₃), 6.92 (t, 1H, J=6.9 Hz, H-b), 7.45 (m, 1H, H-c), 8.10 (d, 1H, J=8.8 Hz, H-d), 8.36 (d, 1H, J=6.8 Hz, H-a). ¹³C NMR (151 MHz, Chloroform-d): δ 57.3 (—OCH₃), 81.1 (C-f), 103.5 (t, J=17.9 Hz), 105.1 (t, J=14.4 Hz), 112.9 (C-b), 117.6 (C-d), 128.3 (C-c)*, 129.2 (C-a)*, 140.8 (C-e), 144.7 (d, J=246.0 Hz), 145.6 (d, J=249.7 Hz), 153.1, 161.2 (C-g), 164.2. (ESI) 443-445 (M+1).

2-(2-Methoxypyrazolo [1,5-a]pyridin-3-yl)-5-(2,3,5,6-tetrafluoro-[1,1′-biphenyl]-4-yl)-1,3,4-oxadiazole (113). K₂CO₃ (0.187 g, 3.0 eq) and Pd(PPh₃)₄ (0.053 g, 0.1 eq) was added to a solution of 112 (0.2 g, 0.45 mmol) in dioxane/H₂O (30 mL, 9/1 v/v) under inert atmosphere. and stirred for 1 h. Then, phenylboronic acid (0.165 g, 3.0 eq.) was added and the reaction mixture was stirred at 90° C. overnight. The reaction mixture was quenched in distilled water (150 mL) and extracted with EtOAc (3×60 mL). Organic layers were collected, washed with a saturated brine solution, dried over Na₂SO₄ and concentrated under reduced pressure to afford a pale, yellow solid. Yield: 100%. ¹⁹F NMR (565 MHz, DMSO-d₆): δ −137.74 (d, 2F, J=21.5 Hz), −142.75 (d, 2F, J=30.2 Hz). ¹H NMR (600 MHz, DMSO-d₆): δ 4.12 (s, 3H, —OCH₃), 7.11 (t, 1H, J=6.8 Hz, H-b), 7.55-7.62 (m, 6H, H-c and aromatic protons), 8.00 (d, 1H, J=8.8 Hz, H-d), 8.78 (d, 1H, J=6.8 Hz, H-a). ¹³C NMR (151 MHz, DMSO-d₆): δ 57.1 (—OCH₃), 79.7 (C-f), 103.5, 113.5 (C-b), 116.4 (C-d), 126.1 (C-c)*, 127.3, 127.4, 128.9, 129.3 (C-a)*, 130.0, 134.1, 139.9 (C-e), 144.7 (d, J=246.0 Hz), 145.6 (d, J=249.7 Hz), 153.0, 160.0 (C-g), 163.3.

3-(5-(2,3,5,6-Tetrafluoro-[1,1′-biphenyl]-4-yl)-1,3,4-oxadiazol-2-yl) pyrazolo [1,5-a]pyridine-2-ol (39). BBr₃ 1M solution in DCM (650 μL, 0.69 mmol) was dissolved in dry DCM (5 mL) under inert atmosphere. The solution was cooled at −10° C. and compound 119 (0.12 g, 0.273 mmol) was added and the reaction mixture was stirred for 30 minutes at 0° C. The reaction was quenched in distilled water (150 mL) and extracted with DCM (3×60 mL). Organic layers were collected, washed with a saturated brine solution, dried over Na₂SO₄ and concentrated under reduced pressure to afford a pale, yellow solid. The crude solid was triturated with diisopropyl ether to afford the title compound as pale, yellow solid. Yield 90%. Degradation temperature: 272-273° C. ¹H NMR (600 MHz, DMSO-d₆): δ 7.06 (t, 1H, J=6.7 Hz, H-b), 7.52-7.65 (m, 6H, H-c and aromatic protons), 7.98 (d, 1H, J=8.7 Hz, H-d), 8.67 (d, 1H, J=6.7 Hz, H-a), 12.05 (s, 1H, —OH). ¹³C NMR (151 MHz, DMSO-d₆): δ 79.5 (C-f), 103.8 (t, J=12.9 Hz), 113.2 (C-b), 116.1 (C-d), 122.6 (t, J=17.1 Hz), 126.1 (C-c)*, 128.52, 128.92, 129.5, 129.9 (C-a)*, 130.0, 139.6 (C-e), 144.7 (d, J=246.0 Hz), 145.6 (d, J=249.7 Hz), 152.8, 160.6 (C-g), 162.8. (ESI) 427 (M+1).

N-(4-Bromo-2,3,5,6-tetrafluorophenyl)-2-((4-methoxybenzyl)oxy)pyrazolo[1,5-a]pyridine-3-carboxamide (114). Oxalyl chloride (811 mg, 6.39 mmol, 3.0 eq.) and dry DMF (10 mL) were added to a cooled (0° C.) solution of 57 (635 mg, 2.13 mmol) in dry THF (49 mL) kept under a nitrogen atmosphere. The resulting mixture was stirred for 2 h at room temperature. In parallel, a 1 M solution of LiHMDS in THF (3.62 mL, 3.62 mmol, 1.7 eq.) was added to a solution of 4-bromo-2,3,5,6-tetrafluoroaniline (883 mg, 3.62 mmol, 1.7 eq.) in dry THF (10 mL) under a nitrogen atmosphere. The resulting suspension was stirred for 30 minutes at room temperature. The solution of acyl chloride was then concentrated under reduced pressure, and the residue was dissolved in dry THF (25 mL); this step was repeated three times to eliminate all gaseous residues). The acyl chloride was dissolved in dry THF (15 mL), and the solution was added to the solution described above. The reaction mixture was stirred at 70° C. overnight, then cooled to room temperature and evaporated. The residue was dissolved in EtOAc (40 mL), 0.5 M HCl (50 mL) was then added, and the layers were separated. The aqueous phase was extracted twice with EtOAc, and the combined organic layers were washed with brine, dried, and evaporated under reduced pressure. The crude material was purified using flash chromatography (eluent: petroleum ether/EtOAc 8/2 v/v and then 7/3 v/v) to afford the title compound as a white solid (melting point=157.9-161.9° C., triturated with diisopropyl ether). Yield: 78%. ¹H-NMR (600 MHz, chloroform-d) δ: 3.82 (s, 3H, —OCH₃), 5.50 (s, 2H, —OCH₂Ar), 6.91 (t, 1H, J=6.9 Hz, H-b), 6.94 (d, 2H, J=8.5 Hz, H-n), 7.41 (d, 1H, J=7.8 Hz, H-c), 7.46 (d, 2H, J=8.5 Hz, H-m), 8.21 (d, 1H, J=8.8 Hz, H-d), 8.26 (s, 1H, —NH), 8.33 (d, 1H, J=6.8 Hz, H-a). ¹³C NMR (151 MHz, chloroform-d) δ: 55.5 (—OCH₃), 72.3 (—OCH₂Ar), 89.8 (C-f), 94.6 (t, J=22.5 Hz, C-s), 113.4 (C-b), 114.3 (C-n), 116.6 (t, J=14.6 Hz, C-p), 118.9 (C-d), 127.6 (C-1), 128.4 (C-a)*, 128.8 (C-c)*, 130.3 (C-m), 142.6 (d, J=251.6 Hz, C-r)**, 143.1 (C-e), 145.2 (d, J=246.5 Hz, C-q)**, 160.19 (C-o)***, 160.24 (C-h)***, 162.9 (C-g)***. ¹⁹F NMR (565 MHz, chloroform-d) −143.55 (d, 2F, J=19.6 Hz, F-p), −133.93 (d, 2F, J=16.6 Hz, F-r). MS (ES⁺): 524/526 (M+1).

1-(3-Bromophenoxy)propan-2-one (116). 3-Chloroacetone (7.70 g, 83.2 mmol, 3.0 eq.) was added dropwise to a mixture of 3-bromophenol (4.80 g, 27.7 mmol) and DBU (16.9 g, 111 mmol, 4.0 eq.) in dry DMF (50 mL). The reaction mixture was stirred at room temperature overnight, then quenched in 2M HCl (250 mL). The mixture was extracted with EtOAc (3×200 mL). The combined organic layers were dried over Na₂SO₄ and concentrated under reduced pressure. The crude material was purified using flash chromatography (eluent: from petroleum ether/EtOAc 9/1 v/v to petroleum ether/EtOAc 8/2 v/v) to afford the title compound as a yellow oil. Yield 980. ¹H NMR (600 MHz, chloroform-d) δ: 2.28 (s, 3H, —CH₃), 4.53 (s, 2H, —OCH₂), 6.82 (ddd, 1H, J=7.9, 2.5, 1.4 Hz, aromatic proton), 7.04-7.06 (m, 1H, aromatic proton) 7.13 (dt, 1H, J=7.9, 1.5 Hz, aromatic proton), 7.16 (d, 1H, J=7.9 Hz, aromatic proton). ¹³C NMR (151 MHz, chloroform-d) δ: 26.8 (—CH³), 72.9 (—OCH₂), 113.6, 118.2, 123.1, 125.1, 130.9, 158.6, 204.9 (—OCH₂ COCH₃). MS (ES⁺): 227/229 (M+1).

1-Bromo-3-(2,2-di fluoropropoxy)benzene (117). DAST (1.40 g, 8.70 mmol, 5.0 eq.) was added dropwise to a solution of 116 (437 mg, 1.74 mmol) in dry DCM (20 mL) at 0° C. The reaction mixture was stirred at room temperature for two hours, then quenched in saturated solution of Na₂CO₃ (30 mL). The mixture was extracted with DCM (3×20 mL). The combined organic layers were dried over Na₂SO₄ and concentrated under reduced pressure. The crude material was purified using flash chromatography (eluent: petroleum ether/EtOAc 8/2 v/v) to afford the title compound as a yellow oil. Yield: 78%. ¹H NMR (600 MHz, chloroform-d) δ: 1.77 (t, 3H, J=18.8 Hz, —CH₃), 4.09 (t, 2H, J=11.3 Hz, —OCH₂), 6.87 (ddd, 1H, J=7.8, 2.4, 1.5 Hz, aromatic proton), 7.09-7.10 (m, 1H, aromatic proton), 7.15 (dt, 1H, J=7.9, 1.5 Hz, aromatic proton); 7.17 (t, 1H, J=7.8 Hz, aromatic proton). ¹³C NMR (151 MHz, chloroform-d) δ: 21.0 (t, J=25.7 Hz, —CH₃), 69.9 (t, J=34.8 Hz, —OCH₂), 113.7, 118.3, 121.4 (t, J=239.3 Hz, —CF₂), 123.0, 125.1, 130.9, 158.7. MS (ES+): 183/185 (M-67). Massa da rifare

3-(3-Bromophenoxy)propan-1-ol (122). PPh₃ (1.80 g, 6.87 mmol, 1.4 eq.) was added to a solution of the 3-bromophenol (1.34 g, 4.91 mmol, 1.0 eq.) and 1,3-propandiol (523 mg, 6.87 mmol, 1.4 eq.) in dry THF (20 mL). After stirring the resulting mixture under a nitrogen atmosphere at r.t. for 30 minutes, DIAD (1.19 g, 6.87 mmol, 1.4 eq.) was added dropwise. The reaction mixture was then stirred at r.t. for 2 days under a nitrogen atmosphere. The mixture was concentrated under reduced pressure and taken-up with 0.1 M NaOH (50 mL). The mixture was extracted with EtOAc (3×20 mL). The combined organic layers were dried over Na₂SO₄ and concentrated under reduced pressure. The crude product was purified by flash chromatography (eluent: petroleum ether/EtOAc 8/2 v/v and then 7/3 v/v). The white solid product was washed with hexane/diisopropyl ether 8/2 v/v and the supernatant liquid was filtrated and concentrated under vacuum obtaining a colourless oil (the procedure was repeated for 3 times). Yield: 69%. ¹H NMR (600 MHz, chloroform-d) δ: 1.83 (br s, —OCH₂CH₂CH₂OH), 2.00-2.06 (m, 2H, —OCH₂CH₂CH₂OH), 3.84 (t, 2H, J=6.0 Hz, OCH₂CH₂ CH₂OH), 4.09 (t, 2H, J=6.0 Hz, —OCH₂CH₂ CH₂OH), 6.83 (ddd, 1H, J=8.3, 2.3, 1.0 Hz, aromatic proton), 7.05-7.09 (m, 2H, aromatic protons), 7.13 (t, 1H, J=7.9 Hz, aromatic proton); ¹³C NMR (151 MHz, chloroform-d) δ: 32.0 (—OCH₂CH₂ CH₂OH), 60.2 (—OCH₂CH₂ CH₂OH), 65.8 (—OCH₂CH₂ CH₂OH), 113.6, 117.9, 122.9, 124.0, 130.7, 159.7; MS (ES+): 271/273 (M+40).

3-(3-Bromophenoxy)propyl tert-butyl carbonate (123). A solution of di tert-butyl carbonate (827 mg, 4.26 mmol, 2.0 eq.) in dry THF (5 mL) was added dropwise to a mixture of 122 (705 mg, 2.13 mmol) and NaH (98.0 mg, 4.26 mmol, 2.0 eq.) in dry THF (10 mL). The reaction mixture was stirred at room temperature overnight, then concentrated under vacuum. The crude material was taken-up with water (50 mL) and the mixture was extracted with EtOAc (3×40 mL). The combined organic layers were dried over Na₂SO₄ and concentrated under reduced pressure. The crude material was purified using flash chromatography (eluent: petroleum ether/EtOAc 9/1 v/v) to afford the title compound as a yellow oil. Yield: 96%. ¹H NMR (600 MHz, chloroform-d) δ: 1.49 (s, 9H, —C(CH₃)₃), 2.10-2.16 (m, 2H, —OCH₂CH₂CH₂O—), 4.04 (t, 2H, J=6.1 Hz, —OCH₂CH₂ CH₂O—), 4.25 (t, 2H, J=6.3 Hz, —OCH₂CH₂CH₂O—), 6.82 (ddd, 1H, J=8.2, 2.4, 0.9 Hz, aromatic proton), 7.04-7.09 (m, 2H, aromatic protons), 7.13 (t, 1H, J=8.0 Hz, aromatic proton). ¹³C NMR (151 MHz, chloroform-d) δ: 27.9 (—C(CH₃)₃), 28.7 (—OCH₂CH₂CH₂O—), 63.7 (—OCH₂CH₂CH₂O—)*, 64.6 (—OCH₂CH₂ CH₂O—)*, 82.3, 85.3 (—C(CH₃)₃), 113.7, 117.9, 122.9, 124.1, 130.7, 146.9, 153.6, 159.7 (CO); MS (ES⁺): 353/355 (M+23).

General Procedure: Synthesis of the Boron Pinacolato Compounds 117, 120, 124. PdCl₂ (dppf) (36.0 mg, 0.0495 mmol, 0.03 eq.) was added to a solution of the corresponding starting material (415 mg, 1.65 mmol) and K₂CO₃ (684 mg, 4.95 mmol, 3.0 eq.) in dioxane (15 mL). After stirring the resulting mixture e under a nitrogen atmosphere for 1 h at r.t., bis (pinacolato)diboron (626 mg, 5.94 mmol 3.60 eq.) was added. The reaction mixture was then heated at reflux overnight under a nitrogen atmosphere. The mixture was cooled to room temperature and concentrated under reduced pressure. The crude material was taken-up with petroleum ether/EtOAc 9/1 v/v (20 mL) and was purified using flash chromatography (see the conditions below).

2-(3-(2,2-Difluoropropoxy)phenyl)-4,4,5,5-tetramethyl-1,3,2-dioxaborolane (117). The crude product was purified by flash chromatography (eluent: petroleum ether/EtOAc 9/1 v/v), obtaining a pale-yellow oil. Yield: 97%. ¹H NMR (600 MHz, chloroform-d) δ: 1.35 (s, 12H, —OC(CH₃)₂), 1.77 (t, 3H, J=18.8 Hz, —OCH₂CF₂CH₃), 4.14 (t, 2H, J=11.4 Hz, —OCH₂CF₂CH₃), 7.04 (ddd, 1H, J=8.2, 2.8, 1.0 Hz, aromatic proton), 7.29-7.35 (m, 2H, aromatic protons), 7.44-7.47 (m, 1H, aromatic proton); ¹³C NMR (151 MHz, chloroform-d) δ: 21.1 (t, J=26.0 Hz, —OCH₂CF₂CH₃), 25.2 (—OC(CH₃)₂), 25.2 (—OC(CH₃)₂), 69.7 (t, J=34.6 Hz, OCH₂CF₂CH₃), 83.6 (—BOC(CH₃)₂C(CH₃)₂O), 84.1 (—B(OC(CH₃)₂C(CH₃)₂₀), 118.3 (t, J=109.9 Hz, OCH₂CF₂CH₃), 118.5, 119.7, 121.7, 128.3, 129.3, 157.5; MS (ES+): 299 (M+1).

1-(3-(4,4,5,5-Tetramethyl-1,3,2-dioxaborolan-2-yl)phenoxy)propan-2-one (120). The crude product was filtered on silica gel (eluent: petroleum ether/EtOAc 9/1 v/v), obtaining a yellow oil. Yield: 96%. ¹H NMR (600 MHz, chloroform-d) δ: 1.24 (s, 6H, —OC(CH₃)₂),), 1.34 (s, 6H, —OC(CH₃)₂), 2.31 (s, 3H, —OCH₂COCH₃), 4.60 (s, 2H, —OCH₂COCH₃), 6.86 (dd, 1H, J=8.2, 2.5 Hz, aromatic proton), 7.09-7.11 (m, 1H, aromatic proton), 7.21 (dd, 1H, J=7.6, 0.5 Hz, aromatic proton), 7.36 (t, 1H, J=7.9 aromatic proton); ¹³C NMR (151 MHz, chloroform-d) δ: 25.0 (—OC(CH₃)₂)), 26.8 (—OCH₂COCH₃), 73.2 (—OCH₂COCH₃), 84.1 (—OC(CH₃)₂), 113.5, 113.8, 118.2, 120.8, 128.3, 130.2, 158.2, 205.8 (—OCH₂ COCH₃). MS (ES+): 277 (M+1).

tert-butyl (3-(3-(4,4,5,5-tetramethyl-1,3,2-dioxaborolan-2-yl) phenoxy) propyl) carbonate (124). The crude product was filtered on silica gel (eluent: petroleum ether/EtOAc 9/1 v/v), obtaining a colourless oil. Yield: 960. ¹H NMR (600 MHz, chloroform-d) δ: 1.34 (s, 12H, —OC(CH₃)₂), 1.47 (s, 9H, —C(CH₃)₃), 2.10-2.14 (m, 2H, —OCH₂CH₂CH₂OC—), 4.08 (t, 2H, J=6.1 Hz, OCH₂CH₂CH₂OC—), 4.25 (t, 2H, J=6.3 Hz, —OCH₂CH₂CH₂OC—), 6.98 (ddd, 1H, J=8.2, 2.7, 0.8 Hz, aromatic proton), 7.27 (t, 1H, J=7.5 Hz, aromatic protons), 7.32 (s, 1H, aromatic proton), 7.38 (d, 1H, J=6.5 Hz, aromatic proton); ¹³C NMR (151 MHz, chloroform-d) δ: 24.9 (—OC(CH₃)₂), 27.9 (—C(CH₃)₃), 28.9 (—OCH₂ CH₂ CH₂OC—), 64.0 (—OCH₂CH₂CH₂OC—), 64.2 ((—OCH₂CH₂CH₂OC—), 82.1 (—C(CH₃)₃), 83.6, 83.9 (—OC(CH₃)₂), 114.6, 118.3, 119.7, 127.3, 129.0, 153.6, 158.3 (—COOC(CH₃)₃). MS (ES+): 401 (M+40).

General Procedure for the synthesis of compounds 119, 121 and 125. Pd(PPh₃) 4 (63.8 mg, 0.0552 mmol, 0.20 eq.) was added to a solution of 114 (145 mg, 0.276 mmol) and K₂CO₃ (114 mg, 0.579 mmol, 3.00 eq.) in dioxane/water mixture (10 mL 9:1 v/v). After stirring the resulting mixture under a nitrogen atmosphere for 1 h at r.t., the corresponding boronic acid pinacol ester (262 mg, 0.579 mmol, 3.0 eq.) was added. The reaction mixture was then heated at reflux under a nitrogen atmosphere. After 5-6 h it was cooled to room temperature and concentrated under reduced pressure. The crude material was taken-up with water (50 mL) and the mixture was extracted with EtOAc (3×40 mL). The combined organic layers were dried over Na₂SO₄ and concentrated under reduced pressure. The crude product was purified by flash chromatography (see the conditions below).

N-(3′-(2,2-Difluoropropoxy)-2,3,5,6-tetra fluoro-[1,1′-biphenyl]-4-yl)-2-((4-methoxybenzyl)oxy) pyrazolo[1,5a] pyridine-3-carboxamide (119). The crude product was purified by flash chromatography (eluent: petroleum ether/EtOAc 8/2 then 7:3 v/v) giving a solid. This solid was then triturated with diisopropyl ether, and the title compound was obtained as a yellow solid (melting point=117.0-114.6° C.). Yield: 79%. ¹H NMR (600 MHz, chloroform-d) δ: 1.80 (t, 3H, J=6.9 Hz, —OCH₂CF₂CH₃), 3.83 (s, 3H, OCH₃), 4.15 (t, 2H, J=11.3, Hz, —OCH₂CF₂CH₃), 5.52 (s, 2H, —CH₂Ar), 6.92 (td, 1H, J=6.9, 1.2 Hz, H-b), 6.96 (d, 2H, J=8.6 Hz, H-n), 7.02-7.05 (m, 2H, J=8.0 Hz, aromatic proton and H-c), 7.10 (d, 1H, J=7.6 Hz, aromatic proton), 7.42 (td, 2H, J=5.21, 8.34, 5.2 Hz, aromatic protons), 7.48 (d, 2H, J=8.6 Hz, H-m), 8.25 (d, 1H, J=8.8 Hz, H-a), 8.32 (s, 1H, NH), 8.34 (d, 1H, J=6.9 Hz, H-d); ¹³C NMR (151 MHz, chloroform-d) δ: 21.1 (t, J=25.7 Hz, —OCH₂CF₂CH₃), 55.4 (—OCH₃), 69.8 (t, J=35.0 Hz, OCH₂CF₂CH₃), 72.3 (—CH₂Ar), 89.9 (C-f), 113.3 (C-b), 114.3 (C-n), 115.5, 116.2 (t, J=14.6 Hz, C-s)*, 116.7, 117.4 (t, J=17.1 Hz, C-p)*, 118.8 (C-d), 121.5 (t, J=239.6 Hz, —OCH₂CF₂CH₃), 123.8, 127.7, 128.3 (C-a), 128.8 (C-e), 128.9 (C-l) 129.9 (C-c), 130.3 (C-m), 142.6 (d, J=237.4 C-r)**, 143.2, 144.13 (d, J=237.4, C-q)**, 158.06 (C-o), 160.18 (C-h), 160.54, 162.96 (C-g); MS (ES+): 616 [M+1], 638 (M+22), 654 (M+39).

2-((4-Methoxybenzyl)oxy)-N-(2,3,5,6-tetrafluoro-3′-(2-oxopropoxy)-[1,1′-biphenyl]-4-yl) pyrazolo[1,5-a]pyridine-3-carboxamide (121). The crude product was purified by flash chromatography (eluent: petroleum ether/EtOAc 8/2 then 7:3 v/v) giving a solid. This solid was then triturated with diisopropyl ether, and the title compound was obtained as a pale-yellow solid (melting point=78.4-81.5° C.). Yield: 70%. ¹H NMR (600 MHz, chloroform-d) δ: 2.31 (s, 3H, —OCH₂COCH₃), 3.83 (s, 3H, —OCH₃), 4.58 (s, 2H, —OCH₂COCH₃), 5.52 (s, 2H, —CH₂Ar), 6.92 (td, 1H, J=7.0 Hz, H-b), 6.93-6.98 (m, 4H, aromatic protons and H-n), 7.09 (d, 1H, J=7.6 Hz, H-c), 7.40-7.43 (m, 1H, aromatic protons), 7.48 (d, 2H, J=7.6 Hz, H-m), 7.48 (d, 2H, J=8.6 Hz, H-n), 8.25 (d, 1H, J=8.8 Hz, H-a), 8.31 (s, 1H, —NH), 8.34 (d, 1H, J=6.9 Hz, H-d); ¹³C NMR (151 MHz, chloroform-d) b: 25.8 (—OCH₂COCH₃), 55.5 (—OCH₃), 72.3 (—OCH₂COCH₃), 73.2 (—CH₂Ar), 89.9 (C-f), 113.3 (C-b), 114.3 (C-n), 115.3, 116.1 (t, J=16.8 Hz, C-s)*, 116.6, 117.3 (t, J=16.3 Hz, C-p)*, 118.8 (C-d), 123.8, 127.7, 128.3 (C-a), 128.8 (C-e), 128.9 (C-1), 130.0 (C-c), 130.3 (C-m), 142.6 (d, J=233.2 Hz, C-r)**, 143.2, 144.2 (d, J=221.7 Hz, C-q)**, 157.9 (C-o), 160.17 (C-h), 160.55, 162.96 (C-g), 205.40 (—OCH₂COCH₃); MS (ES−): 592 (M−1).

Tert-butyl (3-((2′,3′,5′,6′-tetrafluoro-4′-(2-((4-methoxybenzyl)oxy)pyrazolo[1,5-a]pyridine-3-carboxamido)-[1,1′-biphenyl]-3-yl)oxy)propyl)carbonate (125). The crude product was purified by flash chromatography (eluent: petroleum ether/EtOAc 75/25 v/v) giving a solid. This solid was then triturated with diisopropyl ether, and the title compound was obtained as a pale-yellow solid (melting point=65.3-67.1° C.). Yield: 670. ¹H NMR (600 MHz, chloroform-d) δ: 21.48 (s, 9H, —C(CH₃)₃), 2.14-2.18 (m, 2H-OCH₂CH₂CH₂OCOC(CH₃)₃), 3.82 (s, 3H, —OCH₃), 4.09 (t, 2H, J=2.1 Hz, —OCH₂CH₂CH₂OCOC(CH₃)₃), 4.27 (t, 2H, J=6.3 Hz, —OCH₂CH₂ CH₂OCOC(CH₃)₃), 5.52 (s, 2H, —CH₂Ar), 6.91 (t, 1H, J=6.91 Hz, H-c), 6.94-6.99 (m, 4H, H-m and aromatic protons), 7.02 (d, 1H, J=7.5 Hz, aromatic proton), 7.37-7.42 (m, Hz, H-b and aromatic proton), 7.48 (d, 2H, J=8.6 Hz, H-n), 8.25 (d, 1H, J=8.8 Hz, H-a), 8.30 (s, 1H, —NH), 8.34 (d, 1H, J=6.9 Hz, H-d); ¹³C NMR (151 MHz, chloroform-d) δ: 27.9 (—C(CH₃)₃), 55.5 (—OCH₃), 63.9 (OCH₂CH₂ CH₂OCOC(CH₃)₃), 64.5 (—OCH₂CH₂ CH₂OCOC(CH₃)₃), 72.3 (—CH₂Ar), 75.2 (—OCH₂CH₂ CH₂OCOC(CH₃)₃) 82.3 (—C(CH₃)₃), 90.0 (C-f), 113.3 (C-b), 114.3 (C-n), 115.4, 116.3 (C-s)*, 116.3, 115.86 C-p)*, 118.8 (C-d), 122.8, 127.7, 128.3 (C-a), 128.6 (C-1), 128.8 (C-e), 129.7 (C-c), 130.3 (C-m), 144.2 (d, J=235.3 Hz, C-r)**, 145.5 (d, J=221.4 Hz, C-q)**, 153.56, 158.9 (C-o), 160.2 (C-h), 160.6, 163.0 (C-g), 200.7 (—OCH₂CH₂ CH₂OCOC(CH₃)₃). MS (ES−): 640 (M−58).

General Procedure: removal of the 4-methoxybenzyloxy moiety to give final compounds 40-42. Thioanisole (187 mg, 1.51 mmol, 10 eq.) was added to a solution of the corresponding starting material (94.8 mg, 0.151 mmol, 1.0 eq.) in TFA (2 mL). The mixture was stirred at room temperature for 30 min-1 h. The mixture was partially concentrated, and the crude product was taken up with water (20 mL) and the mixture was extracted with EtOAc (3×20 mL). The crude product was purified by flash chromatography (see the conditions below).

2-Hydroxy-N-(2,3,5,6-tetrafluoro-3′-(3-hydroxypropoxy)-[1,1′-biphenyl]-4-yl) pyrazolo[1,5-a]pyridine-3-carboxamide (42). The crude product was purified by flash chromatography (eluent: from DCM to DCM/MeOH 98/2 v/v) giving a solid. The solid underwent to Combiflash Rf 200 purification in reverse phase (eluent: from MeOH/water 35/75 v/v to water after thirteen volumes of column). The title was obtained as a white solid (melting point=X). Yield: 34%. ¹H NMR (600 MHz, DMSO-d6) δ: 1.86-1.90 (m, 2H, —OCH₂CH₂CH₂OH), 3.57 (dd, 2H, J=5.4 Hz, —OCH₂CH₂CH₂OH), 4.09 (t, 2H, J=6.3 Hz, —OCH₂CH₂CH₂OH), 4.57 (br t, 1H, J=3.8, —OCH₂CH₂CH₂OH), 6.96 (t, 1H, J=7.0 Hz, H-b), 7.08-7.11 (m, 3H, aromatic protons), 7.45-7.48 (m, 2H, H-c, aromatic proton), 7.91 (d, 1H, J=7.0 Hz, H-d), 8.55 (d, 1H, J=8.7 Hz, H-d), 9.37 (br s, 1H, —NH), 12.92 (br s, 1H, —OH); NMR da rifare MS (ES−): 474 (M−1).

N-(3′-(2,2-difluoropropoxy)-2,3,5,6-tetrafluoro-[1,1′-biphenyl]-4-yl)-2-hydroxypyrazolo[1,5-a]pyridine-3-carboxamide (40). The crude product was purified by flash chromatography (eluent: from DCM to DCM/EtOAc 95/5 v/v) giving a solid. This solid was then triturated with diisopropyl ether, and the title compound was obtained as a white solid (melting point=229.6-231.7° C.). Yield: 43%. MS (ES+): 494 (M−1). ¹H NMR (600 MHz, DMSO-d6) δ: 1.76 (t, 3H, J=19.2 Hz, —OCH₂CF₂CH₃), 4.36 (t, 2H, J=12.7 Hz, —OCH₂CF₂CH₃), 7.03 (t, 1H, J=6.8 Hz, H-b), 7.17-7.20 (m, 2H, aromatic protons), 7.24 (s, 1H, aromatic proton), 7.50-7.52 (m, 2H, H-c and aromatic proton), 7.98 (d, 1H, J=8.7 Hz, aromatic proton), 8.62 (d, 1H, J=6.7 Hz, H-a), 8.94 (br s, 1H, —NH), 12.83 (br s, 1H, —OH); ¹³C NMR (151 MHz, DMSO-d6) δ: 21.38 (t, J=24.9 Hz, —OCH₂CF₂CH₃), 68.9 (t, J=31.5 Hz, —OCH₂CF₂CH₃), 88.2 (C-f), 113.2 (C-b), 115.8, 116.5 (C-d), 116.8 (t, J=10.6 Hz, C-s)*, 117.8 (C-p)*, 119.9 (t, J=243.5 Hz, —OCH₂CF₂CH₃), 123.3, 128.0, 128.3, 129.1 (C-a), 130.1 (C-c), 141.6 (d, J=269.4 Hz, C-q)**, 141.7 (C-e), 142.8 (d, J=244.7 Hz, C-r)**, 152.6, 157.6 (C-h), 160.4, 170.6 (C-g); ¹⁹F NMR (565 MHz, DMSO-d6) −145.3 (s, 2F, —OCH₂CF₂CH₃), −97.0 (d, 2F, J=14.7 Hz), −96.9 (d, 2F, J=15.0 Hz); MS (ES−): 494 [M−1].

2-Hydroxy-N-(2,3,5,6-tetrafluoro-3′-(2-oxopropoxy)-[1,1′-biphenyl]-4-yl) pyrazolo [1,5-a]pyridine-3-carboxamide (41). The crude product was purified by flash chromatography (eluent: from DCM to DCM/MeOH 95/5 v/v) giving a solid. This solid was then triturated with diisopropyl ether, and the title compound was obtained as a white solid (melting point=218.2-220.3° C.). Yield: 43%. ¹H NMR (600 MHz, DMSO-d₆) δ: 2.17 (s, 3H, —OCH₂COCH₃), 4.90 (s, 2H, —OCH₂COCH₃), 7.02 (t, 1H, J=6.6 Hz, H-b), 7.06 (d, 1H, J=8.5 Hz, aromatic proton), 7.11-7.12 (m, 2H, aromatic protons), 7.46 (t, 1H, J=7.9 Hz, H-c), 7.50 (t, 1H, J=7.8 Hz, aromatic proton), 7.97 (d, 1H, J=8.7 Hz, H-d), 8.60 (d, 1H, J=8.7 Hz, H-a), 8.99 (s, 1H, —NH), 12.90 (br s, 1H, —OH); ¹³C NMR (151 MHz, DMSO-d₆) δ: 26.2 (—OCH₂COCH₃), 72.1 (—OCH₂COCH₃), 88.2 (C-f), 113.2 (C-b), 115.5, 116.2 (C-d), 116.8, 116.9 (C-s)*, 118.1 (C-p)*, 122.7, 127.8, 128.3, 129.1 (C-a), 129.9 (C-c), 140.0 (d, J=246.7 Hz, C-q)**, 141.7 (C-e), 143.80 (d, J=252.7 Hz C-r)**, 157.9, 160.5 (C-h), 162.6 (C-g); 203.8 (—OCH₂ COCH₃). MS (ES−): 472 [M−1]. 2-Hydroxy-N-(2,3,5,6-tetrafluoro-3′-(2-hydroxypropoxy)-[1,1′-biphenyl]-4-yl) pyrazolo[1,5-a]pyridine-3-carboxamide (43). NaBH 4 (8.00 mg, 0.210 mmol, 2 equiv) (5 mL) was added to a mixture of 41 (50.0 mg, 0.105 mmol) in abs EtOH (5 mL). The reaction mixture was stirred at room temperature 1 h, then concentrated under vacuum. The crude material was taken-up with water (10 mL) and the mixture was extracted with EtOAc (3×10 mL). The combined organic layers were dried over Na₂SO₄ and concentrated under reduced pressure. The crude material was purified by trituration with diisopropyl ether, affording a white solid (melting point=175.7-177.6° C.). Yield: 90%. ¹H NMR (600 MHz, DMSO-d₆) δ: 1.16 (d, 3H, J=6.2 Hz, —OCH₂CH(OH)CH₃), 3.86 (td, 2H, J=9.2, Hz, —OCH₂CH(OH)CH₃), 3.94-4.05 (m, 1H, OCH₂CH(OH)CH₃), 4.90 (s, 1H, —OCH₂CH(OH)CH₃), 7.03 (t, 1H, J=6.6 Hz, H-b), 7.09-7.12 (m, 3H, aromatic protons), 7.46 (t, 1H, J=7.9 Hz, H-c), 7.51 (t, 1H, J=7.8 Hz, aromatic proton), 7.98 (d, 1H, J=8.7 Hz, H-d), 8.62 (d, 1H, J=6.7 Hz, H-a), 8.92 (s, 1H, —NH), 12.86 (br s, 1H, acid —OH); ¹³C NMR (151 MHz, DMSO-d6) δ: 20.10 (—OCH₂CH(OH) CH₃) 64.5 (—OCH₂CH(OH) CH₃) 73.4 (—OCH₂CH(OH)CH₃), 88.2 (C-f), 113.2 (C-b), 114.4, 115.6, 116.3 (C-d), 116.9 (C-s)*, 117.5 (C-p)*, 122.3, 127.8, 128.3, 129.2 (C-a), 130.0 (C-c), 141.68 (d, J=242.9 Hz, C-q)**, 141.72 (C-e), 142.8 (d, J=25.3 Hz, C-r)**, 158.7, 160.4 (C-h), 162.7 (C-g); MS (ES−): 474 [M−1].

Protein expression and purification. BL21DE3 PyrD E. Coli cells were transformed using the plasmid construct pFN2A-hDHODH (kindly given by Department of Drug Science and Technology, University of Turin, Turin). The vector produces hDHODH as an N-terminal GST-fusion protein. Cells were grown at 37° C. in LB medium supplemented with mM flavin mononucleotide (Cayman Chemical). After 20 h of growth, cells were induced with 0.8 mM isopropyl-D-thiogalactopyranoside at an OD600 of 0.5-0.7 at 28° C. for an additional 6 h. A cell pellet from 250 mL of culture was lysed in 20 mL of PBS (50 mM Na2HPO4, 50 mM NaH2PO4, 500 mM NaCl), which had been supplemented with 24 mg of lysozyme and 0.2% v/v protease inhibitor cocktail, incubated for 30 min over ice, and disrupted by sonication (total sonication time: 8 minutes with On/Off cycles of 10″/50″). Triton X-100 was added to the lysate, to a final concentration of 1%, before centrifugation at 14000 g for 40 min at 4° C. The clarified supernatant was incubated with DNase I for 30 min at room temperature, supplemented with 2 mM dithiothreitol (DTT), and filtered through a 0.45 μm syringe filter as previously described by Sainas et al.⁶. The GST-fused enzyme was purified from the bacterial lysate using affinity chromatography on immobilized glutathione-sepharose columns (GE-HiTrap Protein G HP 1 ml). The GST tag was not cleaved for further analysis. All the reagents used in the protein expression and purification were supplied by Merck/Sigma-Aldrich, if not otherwise specified.

hDHODH inhibition assay. The enzymatic inhibition assay was optimized for being performed on a 96 well plate and to achieve higher throughput. For each well of the plate a total volume of 200 μL was used: 5 μL of purified GST-hDHODH; 60 μL of 2,6-dichloroindophenol (DCIP) 500 μM; μL of coenzyme Q10 enzyme 100 μM; 20 μL of dihydroorotate (DHO) 500 μM; Tris-HCl pH8 up to a final volume of 200 μL. Inhibitory activity was assessed by monitoring the reduction of DCIP, which is associated with the oxidation of dihydroorotate as catalysed by the DHODH enzyme. The enzyme was pre-incubated for 5 min at 37° C. in Tris-HCl pH8 with coenzyme Q10, with DCIP (50 μM) and with the compounds to be tested used at different concentrations (final DMSO concentration 0.1% v/v). The reaction was initiated by the addition of DHO (500 μM), and the absorbance kinetic reduction was monitored at λ=650 nm using a multi-plate reader (Tecan, M1000Pro). In order to assess the minimum and maximum absorbance values of the enzymatic reaction, a Min control value was obtained by measuring the absorbance without DHO. Similarly, a Max value was obtained by measuring the absorbance with DHO, but none inhibitor. A blank reduction calculation was also performed by measuring the absorbance values using 180 μL of Tris-HCl and 20 μL of coenzyme Q10. The Instrument was set to read the absorbance values every 10 s for a total read time of 10 minutes at 37° C. The initial rate was measured in the first 5 min (s=10 400 M-1 cm-1) and an IC50 value was calculated, n using GraphPad Prism 7 software. Values are means±SE of three independent experiments.

Statistical analysis. Statistical analyses were performed on Prism software, version 5.0 (GraphPad Software, San Diego, CA). Data are reported as means±SD. Two-tail paired Student's t tests were calculated to assess the differences between mean values, and P<0.05 was considered significant. For the determination of EC₅₀, a non-linear regression model was applied.

### Preliminary ADME and Chemophysical Profiling

Solubility assay at pH 7.4. Solubility was assayed in Phosphate Buffered Saline (PBS: 12 mM with NaCl 137 mM and KCl 2.7 mM, pH 7.4). Each solid compound (1 mg) was added to 1 mL of PBS. The samples were shaken in an orbital shaker at 25° C. for 24 h. These suspensions were filtered through a PTFE 0.45 μm filter (VWR), and the solutions were chromatographically analyzed using a Perkin Elmer Ultra High Performance Liquid Chromatography (UHPLC) instrument, equipped with a reverse-phase (RP) C18 Phenomenex column (2.1×100 mm, 1.7 μm particle size). Gradient elution: the ratio of eluents A and B (0.1% trifluoroacetic acid in water and 0.1% trifluoroacetic acid in acetonitrile, respectively) changed linearly from 60% A-40% B to 0% A-100% B in 12 minutes, followed by 5 minutes in isocratic elution at 100% of eluent B and then 4 minutes in equilibration elution to reset the starting conditions. The flow rate was 0.5 mL/min. Standard injection volumes were either 2 or 4 μl for poorly soluble compounds. The detection system was a Perkin Elmer diode-array-detector. The wavelengths that were monitored for each compound were defined according to the compound's own absorption spectrum. Solubility, expressed as μM concentration of the saturated solution, was calculated via interpolation with external calibration curves that were obtained with solutions of each compound in acetonitrile.

Clog P and log D (pH 7.4). ClogP values were calculated using the Bio-Loom program for Windows, Version 1.5 (BioByte). The partition coefficients between n-octanol and PBS at pH 7.4 (log D^{7.4}) were obtained using the shake-flask technique at room temperature. In the shake-flask experiments, 50 mM of phosphate buffered saline pH 7.4 was used as the aqueous phase. The organic (n-octanol) and aqueous phases were mutually saturated by shaking for 4 h. The compounds were solubilized in the buffered aqueous phase at the highest concentration compatible with solubility and appropriate amounts of n-octanol were added. The two phases were shaken for about 20 min, by which time the partitioning equilibrium of solutes had been reached, and then centrifuged (10000 rpm, 10 min). The concentration of the solutes was measured in the aqueous phase using a UV spectrophotometer (Varian Cary absorbance values (recorded for each compound at the wavelength of maximum absorption), were interpolated in calibration curves obtained using standard solutions of the compounds (r²>0.99). Each log D value is an average of at least six measurements.

Protein binding in vitro. This was achieved via ultrafiltration using commercially available membrane systems (Centrifree ultrafiltration devices with ultracel YM-T membrane, Merck). A solution of the selected compound in DMSO was added to human serum (sterile-filtered from human male AB plasma, Sigma-Aldrich), to give the final concentration of 50 μM with 2% of co-solvent. 1 mL of the solution obtained in the sample reservoir of the ultrafiltration device was gently shaken in an orbital shaker at 37° C. for 1 h. The tube was then centrifuged at 1000×g for 15 min. The concentrations of the compounds in the ultrafiltrate and filtrate were determined using reverse-phase UHPLC and the chromatographic conditions were those described above with different injection volumes; 20 μL for ultrafiltrate samples and 2 μL for filtrate samples. The quantitation of the compounds in the filtrate and in ultrafiltrate was performed using two different calibration curves of compound standard solutions (linearity determined in concentration ranges of 0.5-25 μM with injection volume of 20 μL for ultrafiltrate and 10-100 μM with injection volume of 2 μL for filtrate; r²>0.99). The recovery of the ultrafiltration process was calculated in order to discover whether any compound was lost during ultrafiltration, in view of the limited solubility of tested compounds.

Recovery=100×[(vol._{bound}λconc_{bound})+(vol._{unbound}×conc_{unbound})]/vol_{initial serum}×concᵢₙᵢₜᵢₐₗ

vol._{bound}: calculated by dividing the weight of the bound fraction (difference between the weights of the sample reservoir after ultrafiltration and empty), by its density (0.991 g/mL assessed by weighing five replicates of a known volume of the bound fraction).

vol._{unbound}: calculated by dividing the weight of the unbound fraction (difference between the weights of the ultrafiltrate cup after and before ultrafiltration), by its density (0.999 g/mL assessed by weighing five replicates of a known volume of the unbound fraction).

conc_{bound}: calculated using the RP-HPLC method.

conc_{unbound}: calculated using the RP-HPLC method (calibration with standard additions)

Average recovery was 90% for all tested compounds.

Virological Methods:

Herpes Simplex Virus type 1 and 2 (HSV-1/2)

Cells, culture conditions and viruses—African green monkey kidney cells (Vero) (ATCC CCL-81) were cultured in Dulbecco's Modified Eagle Medium (DMEM; Euroclone) supplemented with 10% fetal bovine serum (FBS; Euroclone), 2 mM L-glutamine, 1 mM sodium pyruvate, 100 U/ml penicillin, and 100 mg/ml streptomycin sulfate.

Clinical isolates of HSV-1 and HSV-2 sensitive to acyclovir were kindly provided by Dr. V. Ghisetti (Amedeo di Savoia Hospital, Turin, Italy). HSV-1 and HSV-2 were propagated and titrated by plaque assay on Vero cells as previously described (Terlizzi et al., Antiviral Research 132, 154-164, 2016).

Antiviral Assay—To determine cell viability, Vero cells were exposed to increasing concentrations of 17, 1 or Brequinar. After 3 days of incubation, the number of viable cells was determined using the Cell Titer Glo® Luminescent Cell Viability assay (Promega).

To evaluate the anti-HSV activity of 1, 17 or Brequinar by plaque reduction assays (PRA), Vero cells were seeded in 24-well plates at a density of 70×10³ cells. After 24 h, cells were treated with different concentrations of 17, 1 or Brequinar 1 h prior to infection, and then infected with HSV-1 or HSV-2 (50 PFU/well). Following virus adsorption (2 h at 37 C), cultures were maintained in medium-containing 0.8% methylcellulose (Sigma) plus compounds. At 48 h post infection (h.p.i.), cells were fixed and stained by using 20% ethanol and 1% crystal violet. Plaques were microscopically counted, and the mean plaque counts for each concentration expressed as a percentage of the mean plaque count for the control virus. The number of plaques was plotted as a function of drug concentration; concentration producing 50% reduction in plaque formation (EC 50) was determined as described by Terlizzi et al. (Antiviral Research 132, 154-164, 2016).

Influenza Virus

Cells, culture conditions and viruses—Madin Darby Canine Kidney cells (MDCK, ATCC CCL-34TM) were propagated in DMEM supplemented with 10% fetal bovine serum (FBS; Euroclone), 2 mM L-glutamine, 1 mM sodium pyruvate, 100 U/ml penicillin, and 100 μg/ml streptomycin sulfate. Infections were performed in the presence of 1 μg/ml of trypsin TPCK treated from bovine pancreas (Sigma-Aldrich) and 0.14% of Bovine Serum Albumin (Sigma-Aldrich). The influenza virus strains A/Puerto Rico/8/34 (IAV) (VR-1469) and B B/Lee/40 (IBV) (VR-101) were obtained from ATCC. IAV and IBV were cultured and titrated by plaque assay on MDCK cells as described by Luganini et al., Front. Microbiol. 9:1826, 2018.

Antiviral Assays—Cytoxicity of 1 and Brequinar was determined on MDCK cells after a 72 h of treatment by means of the Cell Titer Glo® Luminescent Cell Viability assay (Promega).

The antiviral activity of 1 and Brequinar was determined by PRA. To this end, MDCK cells were seeded in 24-well plates (3×10⁵ cells/well) and after 24 h they were exposed 1 h prior to infection to increasing concentrations of 1 or Brequinar and then infected with IAV or IBV (40 PFU/well). After virus adsorption (1 h at 37° C.), cultures were incubated in medium containing 0.7% Avicel (FMC BioPolymer) plus 1 or Brequinar. At 48 h post-infection (h p.i.), the cells were fixed with a solution of 4% formaldehyde in phosphate-buffered saline 1× (PBS) for 1 h at room temperature (RT) and stained with a solution of 1% crystal violet. The microscopic plaques count then allowed to define the concentration of either 1 produced 50 reduction in plaque formation (EC₅₀) (Luganini et al., Front. Microbiol. 9:1826, 2018).

Respiratory Syncytial Virus (RSV)

Cells, culture conditions and viruses—HEp-2 cells (ATCC CCL-23) were maintained in Dulbecco's Modified Eagle Medium (DMEM; Euroclone) supplemented with 10% fetal bovine serum (FBS; Euroclone), 2 mM L-glutamine, 1 mM sodium pyruvate, 100 U/ml penicillin, and 100 mg/ml streptomycin sulfate.

The Respiratory Syncytial Virus (RSV) strains A-Long (VR-26) and B-Washington (RSV-9320 VR-955) were obtained from ATCC and propagated and titrated on HEp-2 cells as described by Rameix-Welti et al. Nat. Commun. 5:5104, 2014.

Antiviral Assays—Cytoxicity assays of 1 and Brequinar were performed on HEp-2 cells with the Cell Titer Glo® Luminescent Cell Viability assay (Promega) after 72 h of incubation with the compounds.

The antiviral activity of 1 and Brequinar was determined by PRA. Briefly, HEp-2 cells were seeded in 24-well plates (3×10⁵ cells/well) and after 24 h they were treated with different concentrations of 1 or Brequinar 1 h prior to infection, and then infected with RSV A or B (50 PFU/well). Following virus adsorption (2 h at 37 C), cultures were maintained in medium-containing 0.3% methylcellulose (Sigma) plus compounds. At 96 h post infection (h.p.i.), cells were fixed and stained by using 20% ethanol and 1% crystal violet. Plaques were microscopically counted, and the mean plaque counts for each concentration expressed as a percentage of the mean plaque count for the control virus. The plaques numbers were plotted against the compound concentrations and the EC₅₀ was determined as the compound concentration producing 50% reduction of the plaque numbers.

alpha- and beta-coronavirus. Human lung fibroblasts MRC5 (ATCC CCL-171), the human colorectal carcinoma HCT-8 (ATCC CCL-244), the human lung adenocarcinoma Calu-3 (ATCC HTB-55), and the African green monkey kidney Vero E6 (ATCC CRL-1586) cell lines were purchased from the American Type Culture Collection (ATCC), and maintained in Dulbecco's Modified Eagle Medium (DMEM; Euroclone) supplemented with 10% fetal bovine serum (FBS, Euroclone), 2 mM glutamine, 1 mM sodium pyruvate, 100 U/ml penicillin, and 100 μg/ml streptomycin sulfate (P/S, both from Euroclone).

hCoV-229E (ATCC VR-740) and hCoV-0C43 (ATCC VR-1558) were purchased from ATCC, and propagated and titrated in MRC5 and HCT-8 cells, respectively. SARS-CoV-2 (2019-nCoV/Italy-INMI1) was obtained from EVAg, and propagated and titrated in Vero E6 cells. SARS-CoV-2/01/human/2020/SWE was isolated on Vero E6 cells from a nasopharyngeal sample, cultivated and titrated as previously described [18].

### Cytotoxicity Assays

Cells were seeded in 96-well plates and after 24 h exposed to increasing concentrations of compounds or vehicle (DMSO), as control. After 72 h of incubation, the number of viable cells was determined using either the CellTiter-Glo Luminescent assay (Promega) according to the specifications of the manufacturer, or the MTT method [19].

### Antiviral Assays

To select the mini-library of hDHODH inhibitors, focus forming reduction assays (FFRAs) were performed on HCT-8 cell monolayers treated with the vehicle (DMSO) for with 0.1 μM of the different compounds 1 h prior to and during infection with the hCoV-OC43 (100 PFU/well). At 72 h post-infection (p.i.) cell monolayers were fixed, and subjected to indirect immunoperoxidase staining with a mAb against the hCoV-0C43 N protein (clone. 542-D7; Millipore) (diluted 1:100). Viral foci were microscopically counted, and the mean counts for each drug concentration were expressed as a percentage of the mean plaque counts of control virus (DMSO). To determine the anti-hCoV-229E activity of MEDS433 or brequinar, MRC5 cell monolayers were treated with different concentrations of the compounds 1 h prior to and during infection with hCoV-229E (100 PFU/well). After 72 h p.i., cell viability was measured using CellTiter-Glo assay as a surrogate measurement of the viral cytopathic effect (CPE), as previously described [21]. To measure the anti-SARS-CoV-2 activity of MEDS433 or brequinar, virus yield reduction assay (VRA) was performed with Vero E6 or Calu-3 cells. Briefly, cell monolayers were treated with the vehicle or increasing concentrations of compounds 1 h before and during infection with SARS-CoV-2 (50 or 100 PFU/well). At 48 h p.i., SARS-CoV-2 in cell super-natants was titrated by plaque assay on Vero E6 cells. Compounds concentrations producing 50 and 90% reductions in plaque formation (EC50) were determined as compared to control treatment (DMSO).

SARS-CoV-2

Cells, culture conditions and viruses—Vero E6 cells (ATCC CRL-1586) were maintained in Dulbecco's Modified Eagle Medium (DMEM) supplemented with 10% fetal bovine serum (FBS), 2 mM L-glutamine, 1 mM sodium pyruvate, 100 U/ml penicillin, and 100 mg/ml streptomycin sulfate. SARS-CoV-2 virus strain 2019-nCoV/Italy-INMI1 was obtained from Istituto Nazionale Malattie Infettive “Lazzaro Spallanzani”, and propagated and titrated on Vero E6 cells.

Antiviral assay—Cytoxicity assays of 1 and Brequinar were performed on Vero E6 cells by means of the MTT assay after 72 h of incubation with the compounds.

The antiviral activity of 1 and Brequinar was determined by virus yield reduction assay (VRA). Briefly, Vero E6 cells were seeded in 24-well plates and after 24 h they were treated with different concentrations of 1 or Brequinar 1 h prior to infection, and then infected with SARS-CoV-2 (50 PFU/well). Following virus adsorption (2 h at 37° C.), cultures were maintained in medium-containing compounds. At 72 h post infection (h.p.i.), cell supernatants were harvested and their infectivity was titrated by plaque assay on Vero E6 cell monolayers. Plaques were microscopically counted at 72 h.p.i., and the mean plaque counts for each concentration expressed as a percentage of the mean plaque count for the control virus. The plaques numbers were plotted against the compound concentrations and the EC₅₀ was determined as the compound concentration producing 50% reduction of SARS-CoV-2 infectivity.

### REFERENCES

1. Brown, K. K.; Spinelli, J. B.; Asara, J. M.; Toker, A., Adaptive Reprogramming of De Novo Pyrimidine Synthesis Is a Metabolic Vulnerability in Triple-Negative Breast Cancer. Cancer Discovery 2017, 7 (4), 391-399.
2. Mathur, D.; Stratikopoulos, E.; Ozturk, S.; Steinbach, N.; Pegno, S.; Schoenfeld, S.; Yong, R.; Murty, V. V.; Asara, J. M.; Cantley, L. C.; Parsons, R., PTEN Regulates Glutamine Flux to Pyrimidine Synthesis and Sensitivity to Dihydroorotate Dehydrogenase Inhibition. Cancer Discovery 2017, 7 (4), 380-390.
3. Koundinya, M.; Sudhalter, J.; Courjaud, A.; Lionne, B.; Touyer, G.; Bonnet, L.; Menguy, I.; Schreiber, I.; Perrault, C.; Vougier, S.; Benhamou, B.; Zhang, B.; He, T.; Gao, Q.; Gee, P.; Simard, D.; Castaldi, M. P.; Tomlinson, R.; Reiling, S.; Barrague, M.; Newcombe, R.; Cao, H.; Wang, Y.; Sun, F.; Murtie, J.; Munson, M.; Yang, E.; Harper, D.; Bouaboula, M.; Pollard, J.; Grepin, C.; Garcia-Echeverria, C.; Cheng, H.; Adrian, F.; Winter, C.; Licht, S.; Cornella-Taracido, I.; Arrebola, R.; Morris, A., Dependence on the Pyrimidine Biosynthetic Enzyme DHODH Is a Synthetic Lethal Vulnerability in Mutant KRAS-Driven Cancers. Cell Chemical Biology 2018, 25 (6), 705-717.e11.
4. Okesli, A.; Khosla, C.; Bassik, M. C., Human pyrimidine nucleotide biosynthesis as a target for antiviral chemotherapy. Current Opinion in Biotechnology 2017, 48, 127-134.
5. WO 2019234186 (Boschi D. et al), published on 12 Dec. 2019.
6. Sainas, S.; Pippione, A. C.; Lupino, E.; Giorgis, M.; Circosta, P.; Gaidano, V.; Goyal, P.; Bonanni, D.; Rolando, B.; Cignetti, A.; Ducime, A.; Andersson, M.; Jarva, M.; Friemann, R.; Piccinini, M.; Ramondetti, C.; Buccinna, B.; Al-Karadaghi, S.; Boschi, D.; Saglio, G.; Lolli, M. L., Targeting myeloid differentiation using potent 2-hydroxypyrazolo[1,5-a]pyridine scaffold-based human dihydroorotate dehydrogenase inhibitors. J. Med Chem 2018, 61 (14), 6034-6055.
7. Dorel, R.; Grugel, C. P.; Haydl, A. M., The Buchwald-Hartwig amination after 25 years. Angew Chem Int Ed Engl 2019, 58 (48), 17118-17129.
8. Takahashi, Y.; Hibi, S.; Hoshino, Y.; Kikuchi, K.; Shin, K.; Murata-Tai, K.; Fujisawa, M.; Ino, M.; Shibata, H.; Yonaga, M., Synthesis and structure-activity relationships of pyrazolo[1,5-a]pyridine derivatives: potent and orally active antagonists of corticotropin-releasing factor 1 receptor. J Med Chem 2012, 55 (11), 5255-69.
9. Williams-Noonan, B. J.; Yuriev, E.; Chalmers, D. K., Free energy methods in drug design: Prospects of “alchemical perturbation” in medicinal chemistry. J Med Chem 2018, 61 (3), 638-649.
10. Christian, S.; Merz, C.; Evans, L.; Gradl, S.; Seidel, H.; Friberg, A.; Eheim, A.; Lejeune, P.; Brzezinka, K.; Zimmermann, K.; Ferrara, S.; Meyer, H.; Lesche, R.; Stoeckigt, D.; Bauser, M.; Haegebarth, A.; Sykes, D. B.; Scadden, D. T.; Losman, J. A.; Janzer, A., The novel dihydroorotate dehydrogenase (DHODH) inhibitor BAY 2402234 triggers differentiation and is effective in the treatment of myeloid malignancies. Leukemia 2019, 33 (10), 2403-2415.
11. Gradl, S. N.; Mueller, T.; Ferrara, S.; Sheikh, S. E.; Janzer, A.; Zhou, H.-J.; Friberg, A.; Guenther, J.; Schaefer, M.; Stellfeld, T.; Eis, K.; Kroeber, M.; Nguyen, D.; Merz, C.; Niehues, M.; Stoeckigt, D.; Christian, S.; Zimmermann, K.; Lejeune, P.; Bruening, M.; Meyer, H.; Puetter, V.; Scadden, D. T.; Sykes, D. B.; Seidel, H.; Eheim, A.; Michels, M.; Haegebarth, A.; Bauser, M., Abstract 2: Discovery of BAY 2402234 by phenotypic screening: A human dihydroorotate dehydrogenase (DHODH) inhibitor in clinical trials for the treatment of myeloid malignancies. Cancer Res. 2019, 79 (13 Supplement), 2.
12. Zhou, J.; Quah, J. Y.; Ng, Y.; Chooi, J. Y.; Toh, S. H.; Lin, B.; Tan, T. Z.; Hosoi, H.; Osato, M.; Seet, Q.; Ooi, A. G. L.; Lindmark, B.; McHale, M.; Chng, W. J., ASLAN003, a potent dihydroorotate dehydrogenase inhibitor for differentiation of acute myeloid leukemia. Haematologica 2019, 105 (9), 2286-2297.
13. Waring, M. J., Lipophilicity in drug discovery. Expert Opin Drug Discov 2010, 5 (3), 235-48.
14. Hao, X.; in, X.; Zhang, X.; Ma, B.; Qi, G.; Yu, T.; Han, Z.; Zhu, C., Identification of quinoxalin-2(1H)-one derivatives as a novel class of multifunctional aldose reductase inhibitors. Future Med. Chem. 2019, 11 (23), 2989-3004.
15. Wang, M.; Cao, R.; Zhang, L.; Yang, X.; Liu, J.; Xu, M.; Shi, Z.; Hu, Z.; Zhong, W.; Xiao, G., Remdesivir and chloroquine effectively inhibit the recently emerged novel coronavirus (2019-nCoV) in vitro. Cell Research 2020, 30 (3), 269-271.
16. Sheahan, T. P.; Sims, A. C.; Zhou, S.; Graham, R. L.; Pruijssers, A. J.; Agostini, M. L.; Leist, S. R.; Schafer, A.; Dinnon, K. H., 3rd; Stevens, L. J.; Chappell, J. D.; Lu, X.; Hughes, T. M.; George, A. S.; Hill, C. S.; Montgomery, S. A.; Brown, A. J.; Bluemling, G. R.; Natchus, M. G.; Saindane, M.; Kolykhalov, A. A.; Painter, G.; Harcourt, J.; Tamin, A.; Thornburg, N. J.; Swanstrom, R.; Denison, M. R.; Baric, R. S., An orally bioavailable broad-spectrum antiviral inhibits SARS-CoV-2 in human airway epithelial cell cultures and multiple coronaviruses in mice. Sci Transl Med 2020, 12 (541).
17. Yoon, J.-J.; Toots, M.; Lee, S.; Lee, M.-E.; Ludeke, B.; Luczo, J. M.; Ganti, K.; Cox, R. M.; Sticher, Z. M.; Edpuganti, V.; Mitchell, D. G.; Lockwood, M. A.; Kolykhalov, A. A.; Greninger, A. L.; Moore, M. L.; Painter, G. R.; owen, A. C.; ompkins, S. M.; Fearns, R.; Natchus, M. G.; Plemper, R. K., Orally Efficacious Broad-Spectrum Ribonucleoside Analog Inhibitor of Influenza and Respiratory Syncytial Viruses. Antimicrobial Agents and Chemotherapy 2018, 62 (8), e00766-18.
18. Sainas, S.; Pippione, A. C.; Boschi, D.; Gaidano, V.; Circosta, P.; Cignetti, A.; Dosio, F.; Lolli, M. L., DHODH inhibitors and leukemia: an emergent interest for new myeloid differentiation agents. Drugs Future 2018, 43 (11), 823-834.
19. Giorgis, M.; Lolli, M. L.; Rolando, B.; Rao, A.; Tosco, P.; Chaurasia, S.; Marabello, D.; Fruttero, R.; Gasco, A., 1,2,5-Oxadiazole analogues of leflunomide and related compounds. Eur J Med Chem 2011, 46 (1), 383-92.

1. A method for inhibiting virus replication or for the therapeutic treatment of viral infection in a subject, comprising administering a compound to a subject in need thereof, wherein the compound is selected from the group consisting of:
compounds of Formula (I): 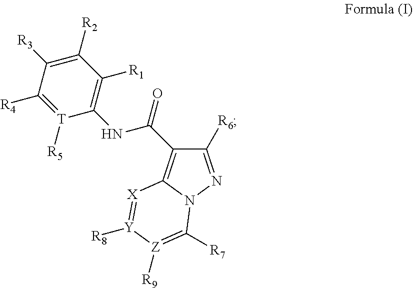
compounds of Formula (II): 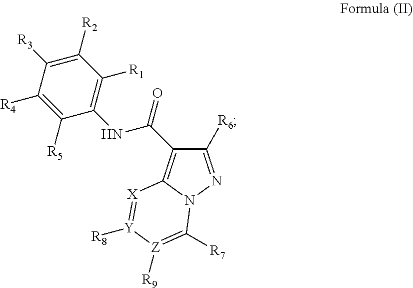
compounds of Formula (III): 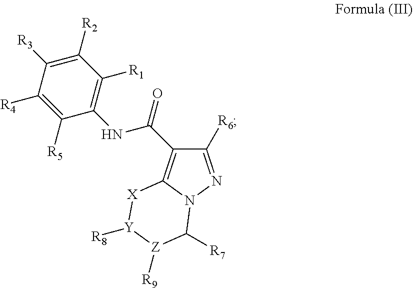
compounds of Formula (IV): 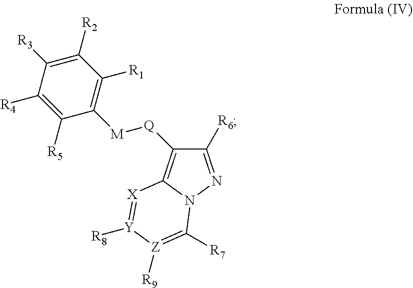
and
compounds of Formula (V): 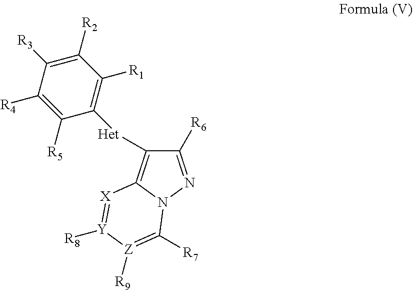
wherein:
R₁, R₂, R₄ and R₅ are independently selected from a hydrogen atom, a halogen atom, a alkyl group, a alkyloxy group, a cycloalkyloxy group, an alkylthio group, a halo alkyl group, a halo alkyloxy group, a nitro group, a cyano group, and alkylamino group;
R₃ in Formulae (I), (II), (III), (IV) and (V) is independently selected from an optionally substituted phenyl group, heteroaryl group, pyridinyl group, piperidinyl group, phenoxy group, pyridinoxy group, piperidinyloxy group, phenylthio group, azinyl group, phenylsulfonyl group, phenylsulfinyl group, phenylsulfonylamino group, alkyl group, alkyloxy group, alkylthio group, halo alkyl group, and halo alkyloxy group;
R₇, R₈ and R₉ in Formulae (I), (II), (III), (IV) and (V) are independently selected from hydrogen atom, a halogen atom, a nitro group, a cyano group, a halo alkyl group, a thio alkyl group, an amino alkyl group, a alkyl group, and a hydroxy alkyl group;
R₆ in Formulae (I), (II), (III), (IV) and (V) is independently selected from a alkyloxy group, a halogen atom, an acyloxy group, a monophosphate group, a hydroxyl group, a thiol group, an amino group, or a salt thereof;
X, Y and Z in Formulae (I), (II), (III), (IV) and (V) are independently selected from a carbon atom, a nitrogen atom, an oxygen atom and a sulfur atom, with the proviso that when one of X, Y or Z is nitrogen, oxygen or sulfur, the other two of X, Y or Z are carbon atoms;
T in Formula (I) is a carbon atom or a nitrogen atom, with the proviso that when T is a nitrogen atom, R₅ in Formula (I) does not exist;
M in Formula (IV) is selected from a carbon sp2 atom, a nitrogen sp3 atom, a nitrogen sp2 atom, a carbonyl group and a sulfonyl group;
Q in Formula (IV) is selected from a carbon sp2 atom, a carbonyl group, a thiocarbonyl group, a sulfonyl group, a polihalogenate-C2-alkylchain, a carbonylamino group, an aminocarbonyl group, a nitrogen sp2 atom and a nitrogen sp3 atom,
with the provisos that when M is a carbon sp2 atom, Q is a carbon sp2 atom; when M is a nitrogen sp3 atom, Q is selected from a carbonyl group, a thiocarbonyl group, a sulfonyl group, a polihalogenate-C2-alkyl chain, a carbonylamino group, an aminocarbonyl group; when M is a nitrogen sp2 atom, Q is a nitrogen sp2 atom; when M is a carbonyl group or sulfonyl group, Q is a nitrogen sp3 atom; and
Het in Formula (V) is selected from azoles, such as an imidazole, a pyrazole, an oxazole, a thiazole, a triazole, an oxadiazole, a thiadiazole, a tetrazole.

2. The method according to claim 1, wherein the alkyls in the definitions of R₁, R₂, R₄ and R₅ are independently selected from the C1-C6 alkyls, preferably from the C1-C4 alkyls.

3. The method according to claim 1, wherein the alkyls in the definitions of R₃ are independently selected from the C1-C12 alkyls.

4. The method according to claim 1, wherein the alkyls in the definitions of R₇, R₈ and R₉ are independently selected from the C1-C6 alkyls, preferably from the C1-C4 alkyls.

5. The method according to claim 1, which is a compound of Formula (I) wherein X=Y=Z are sp2 C atoms and R₉ is a proton.

6. The method according to claim 1, which is a compound of Formula (II) in which X=Y=Z are sp3 C atoms and R₉ is a proton.

7. The method according to any one of claim 1, wherein at least one of R₁, R₂, R₄ and R₅ is or contains a halogen atom, preferably a fluorine atom.

8. The method according to any one of claim 1, wherein R₆ is OH or a monophosphate.

9. The method according to claim 1, wherein the compound is selected from the group consisting of:

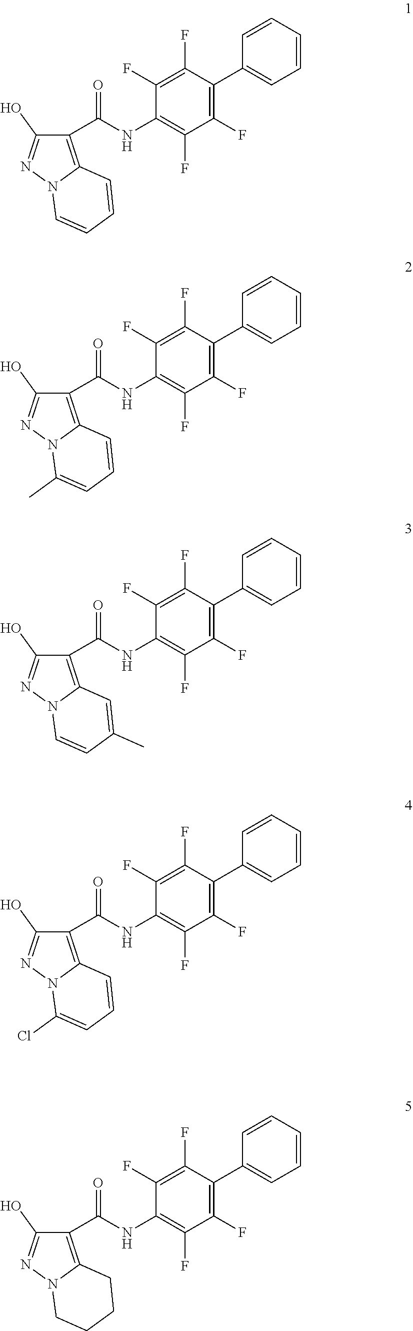 [SMILES: O=C(Nc1c(F)c(F)c(-c2ccccc2)c(F)c1F)c1c(O)nn2ccccc12 ; Cc1cccc2c(C(=O)Nc3c(F)c(F)c(-c4ccccc4)c(F)c3F)c(O)nn12 ; Cc1ccn2nc(O)c(C(=O)Nc3c(F)c(F)c(-c4ccccc4)c(F)c3F)c2c1 ; O=C(Nc1c(F)c(F)c(-c2ccccc2)c(F)c1F)c1c(O)nn2c(Cl)cccc12 ; O=C(Nc1c(F)c(F)c(-c2ccccc2)c(F)c1F)c1c(O)nn2c1CCCC2]

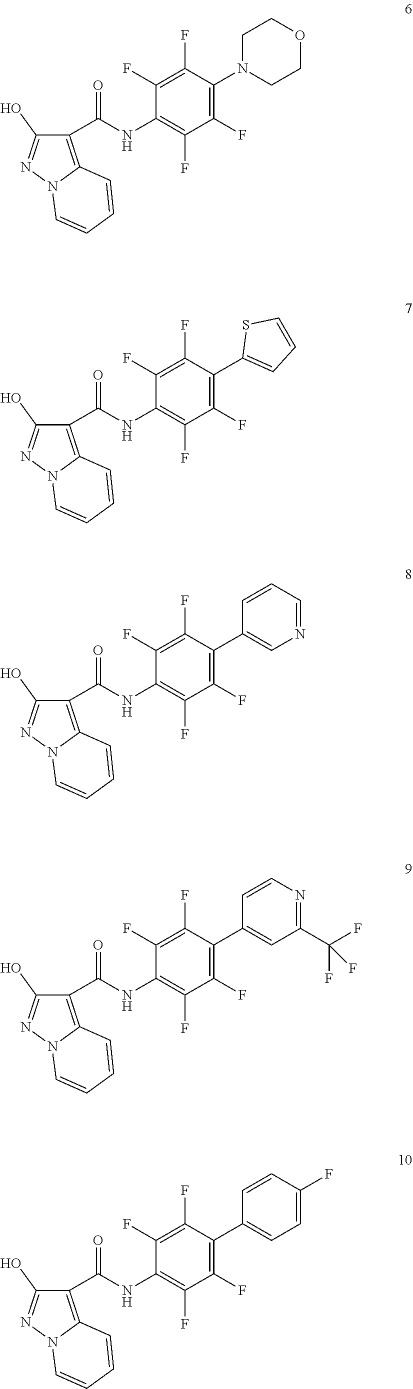 [SMILES: O=C(Nc1c(F)c(F)c(N2CCOCC2)c(F)c1F)c1c(O)nn2ccccc12 ; O=C(Nc1c(F)c(F)c(-c2cccs2)c(F)c1F)c1c(O)nn2ccccc12 ; O=C(Nc1c(F)c(F)c(-c2cccnc2)c(F)c1F)c1c(O)nn2ccccc12 ; O=C(Nc1c(F)c(F)c(-c2ccnc(C(F)(F)F)c2)c(F)c1F)c1c(O)nn2ccccc12 ; O=C(Nc1c(F)c(F)c(-c2ccc(F)cc2)c(F)c1F)c1c(O)nn2ccccc12]

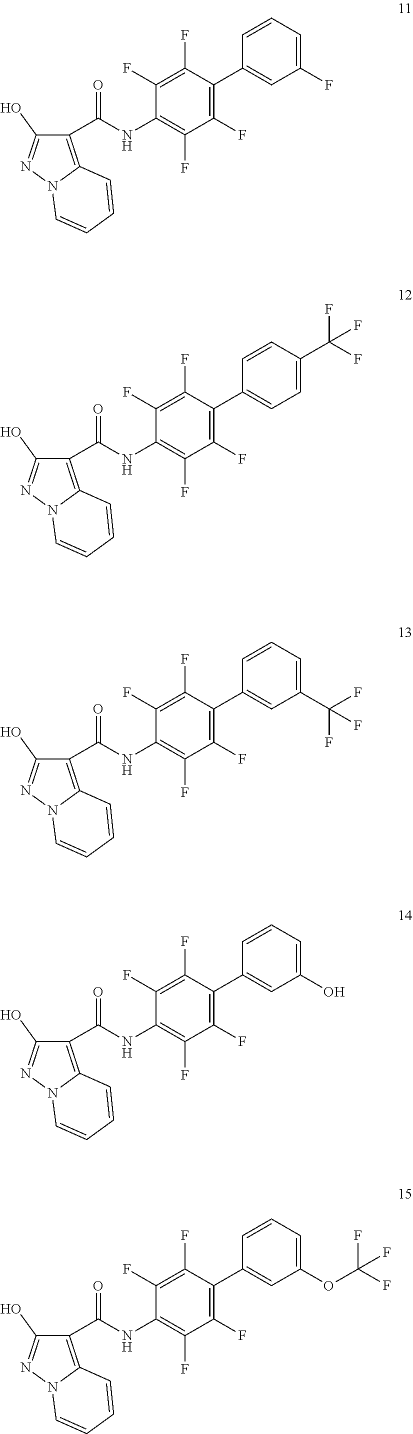 [SMILES: O=C(Nc1c(F)c(F)c(-c2cccc(F)c2)c(F)c1F)c1c(O)nn2ccccc12 ; O=C(Nc1c(F)c(F)c(-c2ccc(C(F)(F)F)cc2)c(F)c1F)c1c(O)nn2ccccc12 ; O=C(Nc1c(F)c(F)c(-c2cccc(C(F)(F)F)c2)c(F)c1F)c1c(O)nn2ccccc12 ; O=C(Nc1c(F)c(F)c(-c2cccc(O)c2)c(F)c1F)c1c(O)nn2ccccc12 ; O=C(Nc1c(F)c(F)c(-c2cccc(OC(F)(F)F)c2)c(F)c1F)c1c(O)nn2ccccc12]

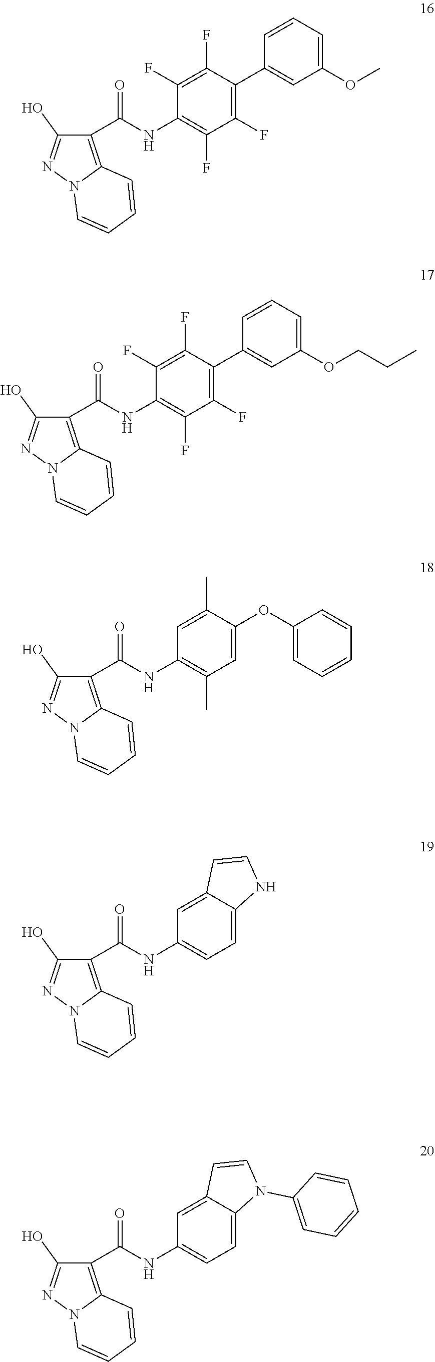 [SMILES: COc1cccc(-c2c(F)c(F)c(NC(=O)c3c(O)nn4ccccc34)c(F)c2F)c1 ; CCCOc1cccc(-c2c(F)c(F)c(NC(=O)c3c(O)nn4ccccc34)c(F)c2F)c1 ; Cc1cc(Oc2ccccc2)c(C)cc1NC(=O)c1c(O)nn2ccccc12 ; O=C(Nc1ccc2[nH]ccc2c1)c1c(O)nn2ccccc12 ; O=C(Nc1ccc2c(ccn2-c2ccccc2)c1)c1c(O)nn2ccccc12]

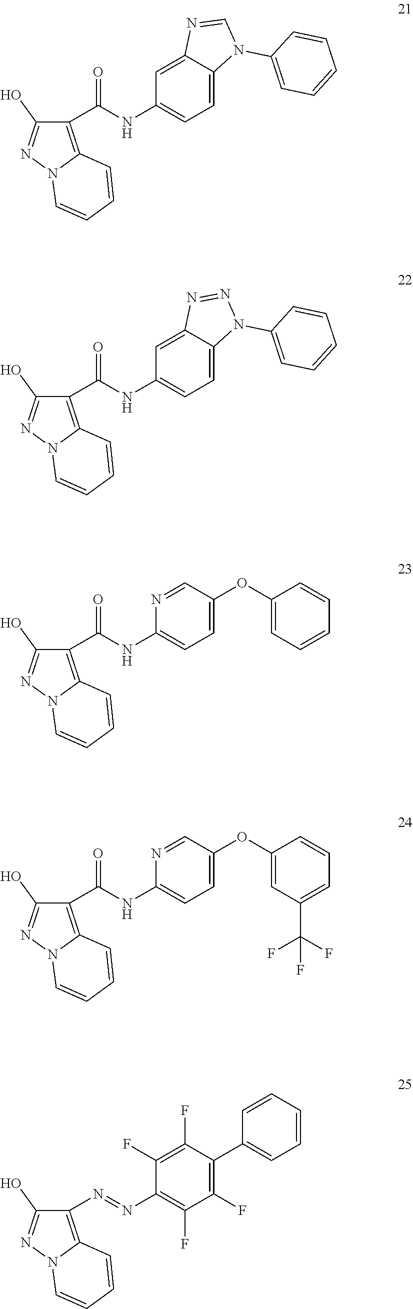 [SMILES: O=C(Nc1ccc2c(c1)ncn2-c1ccccc1)c1c(O)nn2ccccc12 ; O=C(Nc1ccc2c(c1)nnn2-c1ccccc1)c1c(O)nn2ccccc12 ; O=C(Nc1ccc(Oc2ccccc2)cn1)c1c(O)nn2ccccc12 ; O=C(Nc1ccc(Oc2cccc(C(F)(F)F)c2)cn1)c1c(O)nn2ccccc12 ; Oc1nn2ccccc2c1/N=N/c1c(F)c(F)c(-c2ccccc2)c(F)c1F]

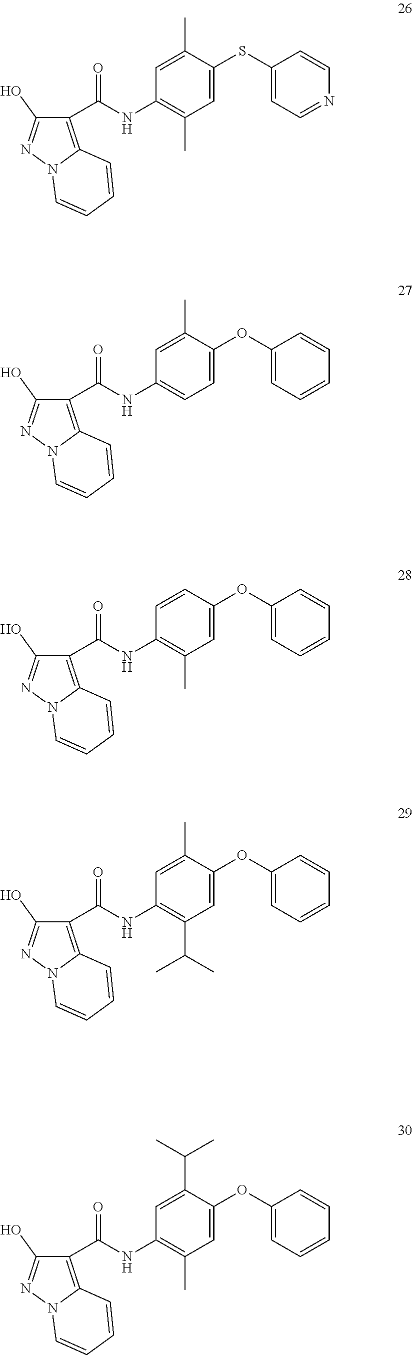 [SMILES: Cc1cc(Sc2ccncc2)c(C)cc1NC(=O)c1c(O)nn2ccccc12 ; Cc1cc(NC(=O)c2c(O)nn3ccccc23)ccc1Oc1ccccc1 ; Cc1cc(Oc2ccccc2)ccc1NC(=O)c1c(O)nn2ccccc12 ; Cc1cc(NC(=O)c2c(O)nn3ccccc23)c(C(C)C)cc1Oc1ccccc1 ; Cc1cc(Oc2ccccc2)c(C(C)C)cc1NC(=O)c1c(O)nn2ccccc12]

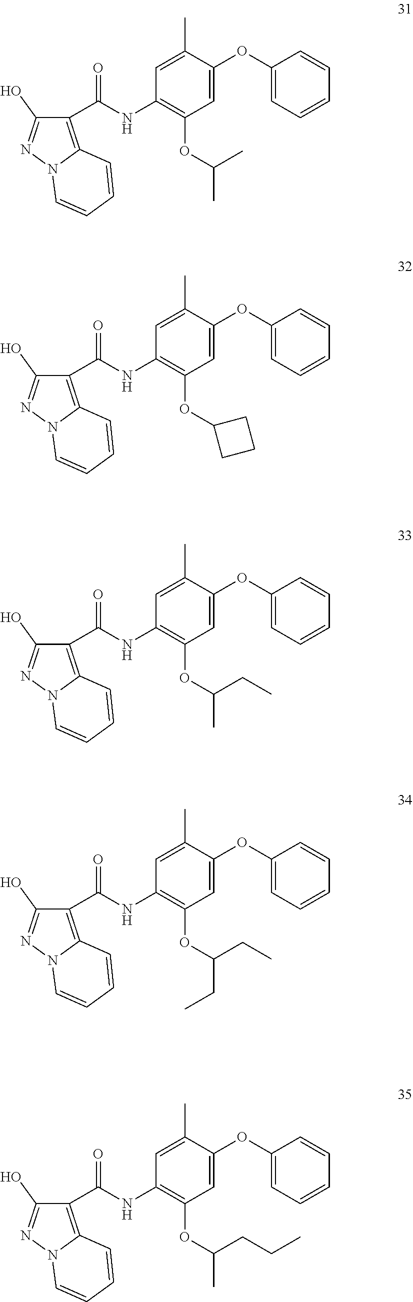 [SMILES: Cc1cc(NC(=O)c2c(O)nn3ccccc23)c(OC(C)C)cc1Oc1ccccc1 ; Cc1cc(NC(=O)c2c(O)nn3ccccc23)c(OC2CCC2)cc1Oc1ccccc1 ; CCC(C)Oc1cc(Oc2ccccc2)c(C)cc1NC(=O)c1c(O)nn2ccccc12 ; CCC(CC)Oc1cc(Oc2ccccc2)c(C)cc1NC(=O)c1c(O)nn2ccccc12 ; CCCC(C)Oc1cc(Oc2ccccc2)c(C)cc1NC(=O)c1c(O)nn2ccccc12]

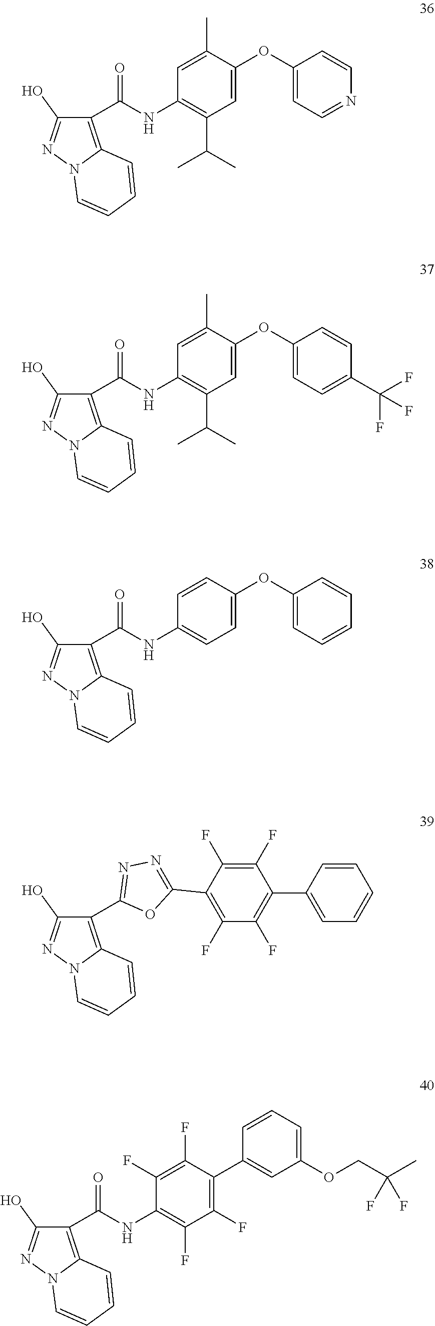 [SMILES: Cc1cc(NC(=O)c2c(O)nn3ccccc23)c(C(C)C)cc1Oc1ccncc1 ; Cc1cc(NC(=O)c2c(O)nn3ccccc23)c(C(C)C)cc1Oc1ccc(C(F)(F)F)cc1 ; O=C(Nc1ccc(Oc2ccccc2)cc1)c1c(O)nn2ccccc12 ; Oc1nn2ccccc2c1-c1nnc(-c2c(F)c(F)c(-c3ccccc3)c(F)c2F)o1 ; CC(F)(F)COc1cccc(-c2c(F)c(F)c(NC(=O)c3c(O)nn4ccccc34)c(F)c2F)c1]

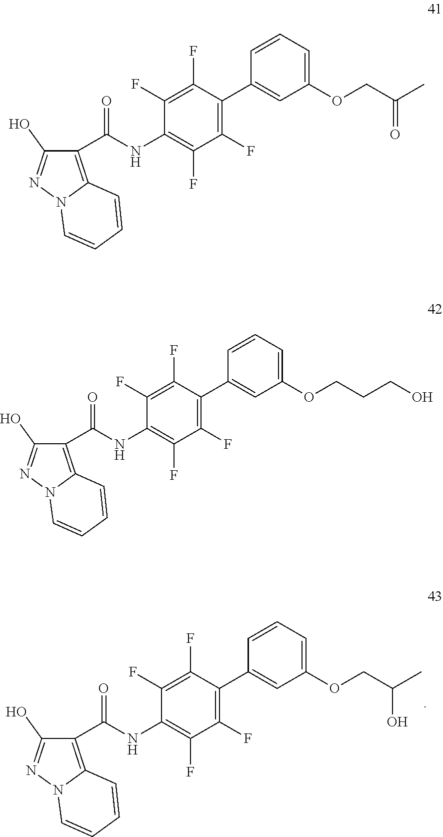 [SMILES: CC(=O)COc1cccc(-c2c(F)c(F)c(NC(=O)c3c(O)nn4ccccc34)c(F)c2F)c1 ; O=C(Nc1c(F)c(F)c(-c2cccc(OCCCO)c2)c(F)c1F)c1c(O)nn2ccccc12 ; CC(O)COc1cccc(-c2c(F)c(F)c(NC(=O)c3c(O)nn4ccccc34)c(F)c2F)c1]

10. The method according to claim 1, wherein the virus is a DNA or a RNA virus.

11. The method according to claim 1, wherein the virus is selected from the group consisting of Herpesviridae, Orthomyxoviridae, Paramyxoviridae and Coronaviridae.

12. The method according to claim 1, wherein the virus is selected from the group consisting of Herpes simplex virus 1 (HSV-1), Herpes simplex virus 2 (HSV-2), Influenza A virus, Influenza B virus, Respiratory syncytial virus (RSV), Severe acute respiratory syndrome coronavirus 1 (SARS-CoV-1), Severe acute respiratory syndrome coronavirus 2 (SARS-CoV-2) and Middle East respiratory syndrome-related coronavirus (MERS-CoV).

13-15. (canceled)

16. A compound selected from the group consisting of:
a compound of Formula (I): 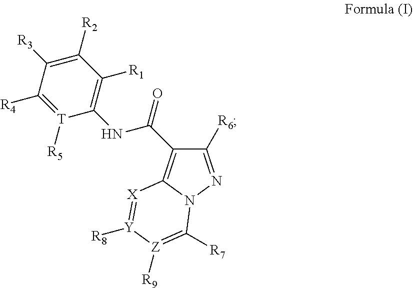
a compound of Formula (IV): 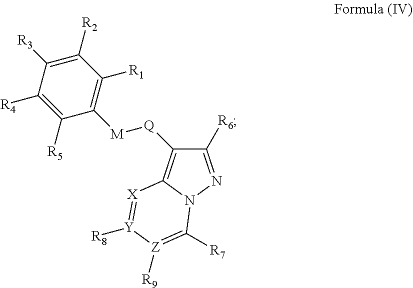
and
a compound of Formula (V): 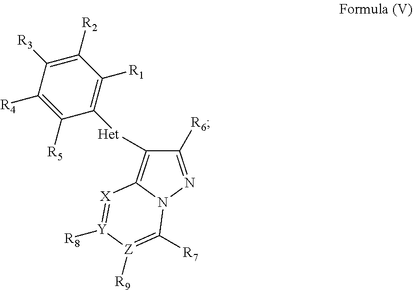
wherein:
R₁, R₂, R₄ and R₅ are independently selected from a hydrogen atom, a halogen atom, a alkyl group, a alkyloxy group, a cycloalkyloxy group, an alkylthio group, a halo alkyl group, a halo alkyloxy group, a nitro group, a cyano group, and alkylamino group;
R₃ in Formulae (I), (IV) and (V) is independently selected from an optionally substituted phenyl group, heteroaryl group, pyridinyl group, piperidinyl group, phenoxy group, pyridinoxy group, piperidinyloxy group, phenylthio group, azinyl group, phenylsulfonyl group, phenylsulfinyl group, phenylsulfonylamino group, alkyl group, alkyloxy group, alkylthio group, halo alkyl group, and halo alkyloxy group;
R₇, R₈ and R₉ in Formulae (I), (IV) and (V) are independently selected from hydrogen atom, a halogen atom, a nitro group, a cyano group, a halo alkyl group, a thio alkyl group, an amino alkyl group, a alkyl group, and a hydroxy alkyl group;
R₆ in Formulae (I), (IV) and (V) is independently selected from a alkyloxy group, a halogen atom, an acyloxy group, a monophosphate group, a hydroxyl group, a thiol group, an amino group, or a salt thereof;
X, Y and Z in Formulae (I), (IV) and (V) are independently selected from a carbon atom, a nitrogen atom, an oxygen atom and a sulfur atom, with the proviso that when one of X, Y or Z is nitrogen, oxygen or sulfur, the other two of X, Y or Z are carbon atoms;
T in Formula (I) is a carbon atom or a nitrogen atom, with the proviso that when T is a nitrogen atom, R₅ in Formula (I) does not exist;
M in Formula (IV) is selected from a carbon sp2 atom, a nitrogen sp3 atom, a nitrogen sp2 atom, a carbonyl group and a sulfonyl group;
Q in Formula (IV) is selected from a carbon sp2 atom, a carbonyl group, a thiocarbonyl group, a sulfonyl group, a polihalogenate-C2-alkylchain, a carbonylamino group, an aminocarbonyl group, a nitrogen sp2 atom and a nitrogen sp3 atom,
with the provisos that when M is a carbon sp2 atom, Q is a carbon sp2 atom; when M is a nitrogen sp3 atom, Q is selected from a carbonyl group, a thiocarbonyl group, a sulfonyl group, a polihalogenate-C2-alkyl chain, a carbonylamino group, an aminocarbonyl group; when M is a nitrogen sp2 atom, Q is a nitrogen sp2 atom; when M is a carbonyl group or sulfonyl group, Q is a nitrogen sp3 atom; and
Het in Formula (V) is selected from azoles, such as an imidazole, a pyrazole, an oxazole, a thiazole, a triazole, an oxadiazole, a thiadiazole, a tetrazole.

17. The compound according to claim 16, wherein the alkyls in the definitions of R₁, R₂, R₄ and R₅ are independently selected from the C1-C6 alkyls, preferably from the C1-C4 alkyls.

18. The compound according to claim 16, wherein the alkyls in the definitions of R₃ are independently selected from the C1-C12 alkyls.

19. The compound according to claim 16, wherein the alkyls in the definitions of R₇, R₈ and R₉ are independently selected from the C1-C6 alkyls, preferably from the C1-C4 alkyls.

20. The compound according to claim 16, which is a compound of Formula (I) wherein X=Y=Z are sp2 C atoms and R₉ is a proton.

21. The compound according to claim 16, wherein at least one of R₁, R₂, R₄ and R₆ is or contains a halogen atom, preferably a fluorine atom.

22. The compound according to claim 16, wherein R₆ is OH or a monophosphate.

23. The compound according to claim 16, which is selected from the group consisting of: 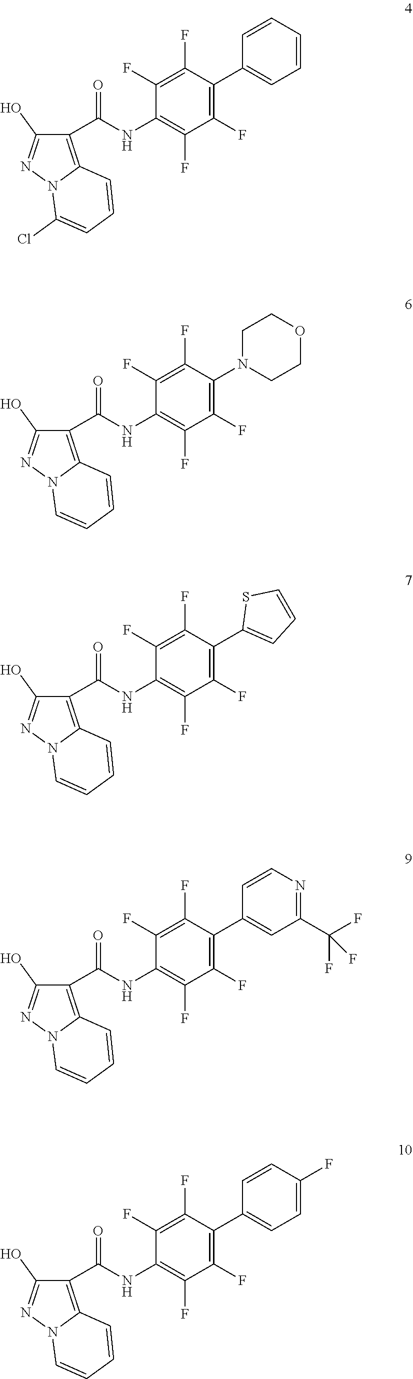 [SMILES: O=C(Nc1c(F)c(F)c(-c2ccccc2)c(F)c1F)c1c(O)nn2c(Cl)cccc12 ; O=C(Nc1c(F)c(F)c(N2CCOCC2)c(F)c1F)c1c(O)nn2ccccc12 ; O=C(Nc1c(F)c(F)c(-c2cccs2)c(F)c1F)c1c(O)nn2ccccc12 ; O=C(Nc1c(F)c(F)c(-c2ccnc(C(F)(F)F)c2)c(F)c1F)c1c(O)nn2ccccc12 ; O=C(Nc1c(F)c(F)c(-c2ccc(F)cc2)c(F)c1F)c1c(O)nn2ccccc12] 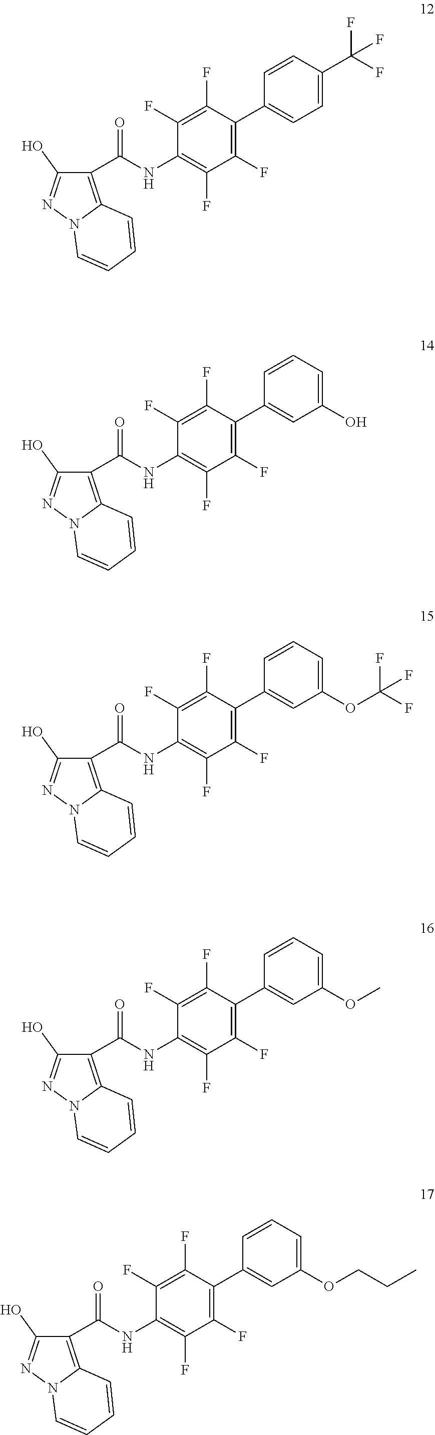 [SMILES: O=C(Nc1c(F)c(F)c(-c2ccc(C(F)(F)F)cc2)c(F)c1F)c1c(O)nn2ccccc12 ; O=C(Nc1c(F)c(F)c(-c2cccc(O)c2)c(F)c1F)c1c(O)nn2ccccc12 ; O=C(Nc1c(F)c(F)c(-c2cccc(OC(F)(F)F)c2)c(F)c1F)c1c(O)nn2ccccc12 ; COc1cccc(-c2c(F)c(F)c(NC(=O)c3c(O)nn4ccccc34)c(F)c2F)c1 ; CCCOc1cccc(-c2c(F)c(F)c(NC(=O)c3c(O)nn4ccccc34)c(F)c2F)c1] 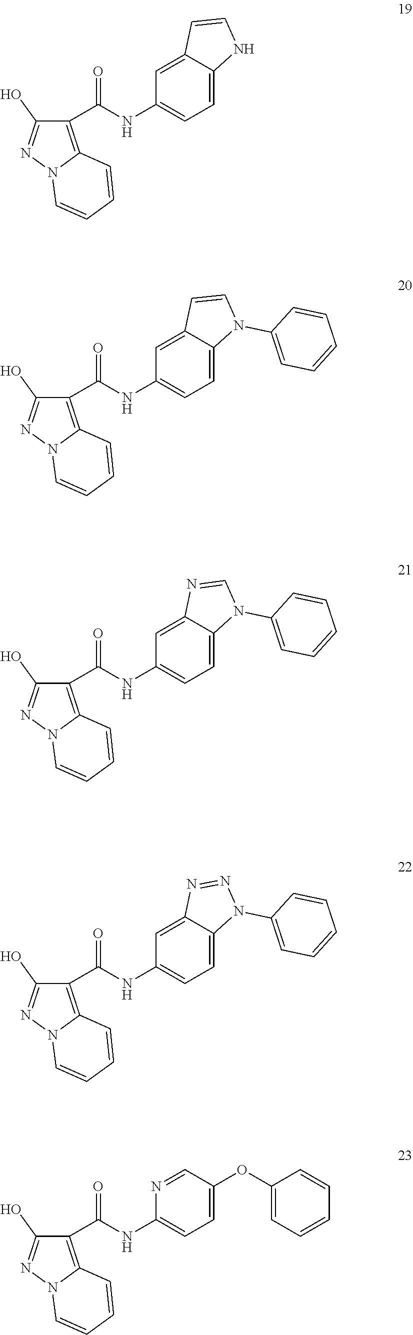 [SMILES: O=C(Nc1ccc2[nH]ccc2c1)c1c(O)nn2ccccc12 ; O=C(Nc1ccc2c(ccn2-c2ccccc2)c1)c1c(O)nn2ccccc12 ; O=C(Nc1ccc2c(c1)ncn2-c1ccccc1)c1c(O)nn2ccccc12 ; O=C(Nc1ccc2c(c1)nnn2-c1ccccc1)c1c(O)nn2ccccc12 ; O=C(Nc1ccc(Oc2ccccc2)cn1)c1c(O)nn2ccccc12] 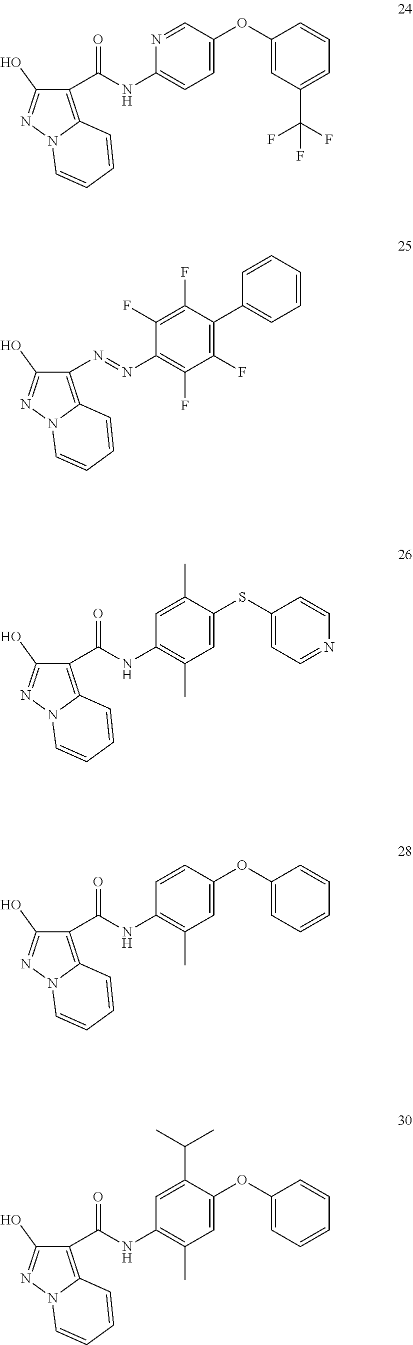 [SMILES: O=C(Nc1ccc(Oc2cccc(C(F)(F)F)c2)cn1)c1c(O)nn2ccccc12 ; Oc1nn2ccccc2c1/N=N/c1c(F)c(F)c(-c2ccccc2)c(F)c1F ; Cc1cc(Sc2ccncc2)c(C)cc1NC(=O)c1c(O)nn2ccccc12 ; Cc1cc(Oc2ccccc2)ccc1NC(=O)c1c(O)nn2ccccc12 ; Cc1cc(Oc2ccccc2)c(C(C)C)cc1NC(=O)c1c(O)nn2ccccc12] 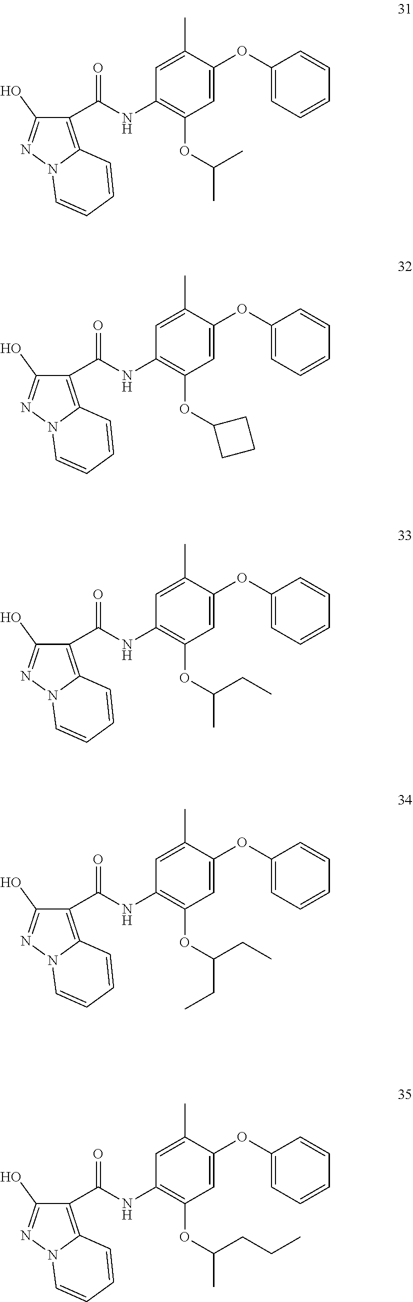 [SMILES: Cc1cc(NC(=O)c2c(O)nn3ccccc23)c(OC(C)C)cc1Oc1ccccc1 ; Cc1cc(NC(=O)c2c(O)nn3ccccc23)c(OC2CCC2)cc1Oc1ccccc1 ; CCC(C)Oc1cc(Oc2ccccc2)c(C)cc1NC(=O)c1c(O)nn2ccccc12 ; CCC(CC)Oc1cc(Oc2ccccc2)c(C)cc1NC(=O)c1c(O)nn2ccccc12 ; CCCC(C)Oc1cc(Oc2ccccc2)c(C)cc1NC(=O)c1c(O)nn2ccccc12] 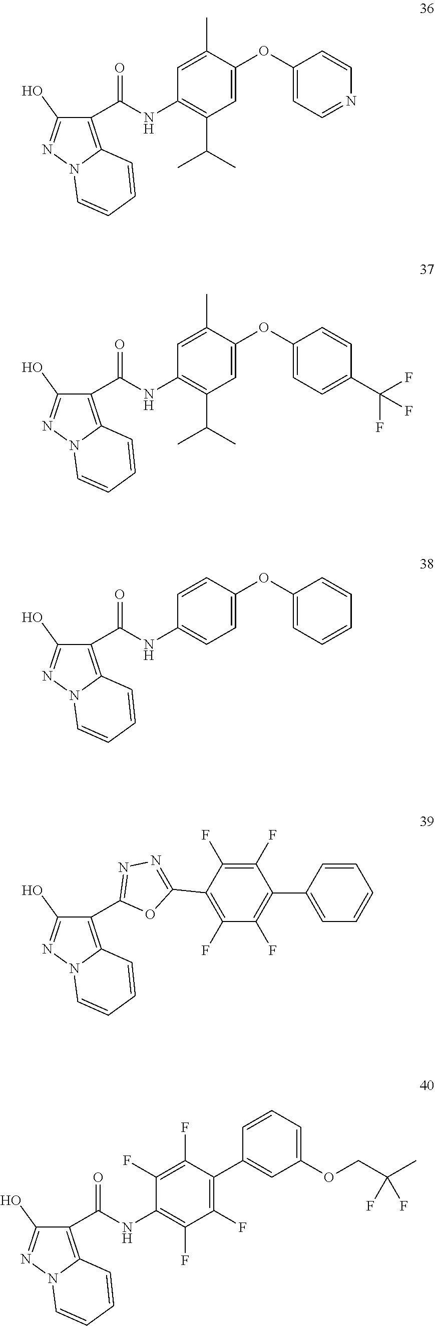 [SMILES: Cc1cc(NC(=O)c2c(O)nn3ccccc23)c(C(C)C)cc1Oc1ccncc1 ; Cc1cc(NC(=O)c2c(O)nn3ccccc23)c(C(C)C)cc1Oc1ccc(C(F)(F)F)cc1 ; O=C(Nc1ccc(Oc2ccccc2)cc1)c1c(O)nn2ccccc12 ; Oc1nn2ccccc2c1-c1nnc(-c2c(F)c(F)c(-c3ccccc3)c(F)c2F)o1 ; CC(F)(F)COc1cccc(-c2c(F)c(F)c(NC(=O)c3c(O)nn4ccccc34)c(F)c2F)c1] 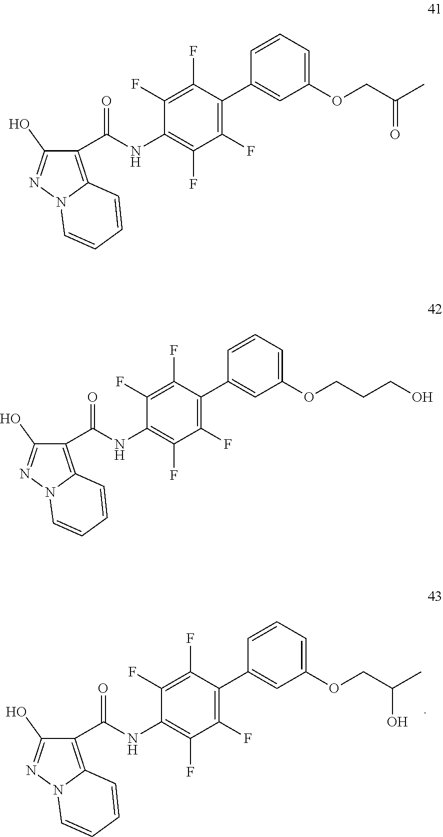 [SMILES: CC(=O)COc1cccc(-c2c(F)c(F)c(NC(=O)c3c(O)nn4ccccc34)c(F)c2F)c1 ; O=C(Nc1c(F)c(F)c(-c2cccc(OCCCO)c2)c(F)c1F)c1c(O)nn2ccccc12 ; CC(O)COc1cccc(-c2c(F)c(F)c(NC(=O)c3c(O)nn4ccccc34)c(F)c2F)c1]
